(19) United States
(12) Patent Application Publication
(10) Pub. No.: US 2010/0291074 A1
(43) Pub. Date: Nov. 18, 2010

(54) CYCLOPHILIN D-AMYLOID BETA INTERACTION POTENTIATES MITOCHONDRIAL DYSFUNCTION IN A TRANSGENIC MOUSE MODEL OF ALZHEIMER'S DISEASE

(75) Inventors: Shi Du Yan, Tenafly, NJ (US); Heng Du, Fort Lee, NJ (US)

Correspondence Address: DITTHAVONG MORI & STEINER, P.C., 918 Prince Street, Alexandria, VA, 22314, US

(73) Assignee: COLUMBIA UNIVERSITY IN THE CITY OF NEW YORK, NEW YORK (US)

(21) Appl. No.: 12/670,827

(22) PCT Filed: Jul. 26, 2008
(86) PCT No.: PCT/US08/71278

§ 371 Date: Jun. 28, 2010

Related U.S. Application Data
(60) Provisional application No. 60/952,533, filed on Jul. 27, 2007.

(51) Int. Cl. A61K 39/395; A61K 38/13; A61P 25/16; A61P 25/28; A61K 31/7088; C07K 14/435; G01N 33/53; C07K 16/18

(52) U.S. Cl. 424/133.1; 514/17.8; 514/17.7; 424/130.1; 424/141.1; 514/44 R; 424/139.1; 530/317; 435/7.92; 530/387.9; 530/387.3

(57) ABSTRACT

The present invention is directed to methods for treating or preventing Alzheimer's disease by administering therapeutically effective amounts of an agent that reduces Cyclophilin D expression in a patient, or that reduce Cyclophilin D activity or its ability to form a complex with Amyloid beta. Such agents include antisense nucleotides and small interfering RNAs, antibodies that selectively bind to Cyclophilin D, and cyclosporine A and D.

### CROSS-REFERENCE TO RELATED APPLICATIONS

This application claims benefit of Provisional Application No. 60/952533, filed Jul. 27, 2007, the entire contents of which are hereby incorporated by reference as if fully set forth herein, under 35 U.S.C. §119(e).

### STATEMENT OF GOVERNMENT INTEREST

This invention was made with Government support under Grant No. NIA PPG17490. The Government has certain rights in the invention.

### BACKGROUND OF THE INVENTION

1. Field of the Invention

The invention is in the field of treatment and prevention of Alzheimer's disease.

2. Description of the Related Art

Alzheimer's disease (AD) is a neurodegenerative disorder of the central nervous system leading to progressive dementia. The disease is characterized by progressive memory loss and the decline of other higher cognitive functions. Approximately 1 out of 10 people of age 65 and over suffer from mild to moderate dementia. The disease is accompanied by a constellation of neuropathologic features principal amongst which are the presence of extracellular amyloid or senile plaques, and neurofibrillary tangles in neurons. The etiology of this disease is complex, although in some families it appears to be inherited as an autosomal dominant trait.

Mitochondrial dysfunction is a hallmark of amyloid beta (Aβ)-induced neuronal perturbation in Alzheimer's disease (Alzheimer's disease). Abnormalities of mitochondrial function, such as decreased activity of respiratory chain enzymes, generation of reactive oxygen species (ROS), and hypometabolism, occur in the Alzheimer's disease (Alzheimer's disease) brain [1-5]. Recent studies demonstrate that progressive accumulation of Aβ in mitochondria is associated with mitochondrial abnormalities in the Alzheimer's disease brain and Alzheimer's disease-type mouse model, [6-11]. However, the mechanisms underlying Aβ-mediated neuronal and mitochondrial toxicity are yet to be elucidated. Therefore there is a great need to understand these mechanisms in order to develop new therapies to prevent and treat mitochondrial toxicity in Alzheimer's disease.

### BRIEF DESCRIPTION OF THE DRAWINGS

The present invention is illustrated by way of example, and not by way of limitation, in the figures of the accompanying drawings and in which:

FIG. 1. (A) Expression of CypD in human AD/Non-Demented (ND) brain tissues and Tg mAPP mice. Mitochondria were isolated from the temporal cortex (A) and hippocampus (B) of AD patients (n=10) and age-matched ND controls (n=8), and from cerebral cortex of Tg mAPP mice (n=9) and nonTg littermate controls (n=8) at 12-month-old age (D). Mitochondria protein (30 μg/lane) was subjected to the SDS-PAGE and immunoblotted with anti-CypD IgG. The intensity of the immunoreactive CypD bands was quantified using computer soft program (NIH image). Immunoblotting of anti-COX IV showed an equal amount of mitochondrial protein loading in each lane. (C). Quantitative real-time PCR analysis of CypD transcripts in the cerebral cortex of Tg mAPP mice (n=9) and nonTg littermate controls (n=8) at 12-month-old age.

FIG. 2. Interaction of CypD with Aβ in vitro and in vivo mitochondria of AD patients and transgenic mice. (A). SDS-PAGE (12%; reduced) of the purified recombinant human CypD protein (5 μg) followed by Coomassie blue staining (lane 1) and by immunoblotting with anti-CypD IgG (1 μg/ml, lane 2). (B-C). Binding affinity of CypD with different species of Aβ by SPR. Different concentrations of CypD were injected in the 90 s association time, and the dissociation time was 120 s. Globally fit data (red lines) using a conformational change model were overlaid with experimental data (black lines). The dissociation constant (KD) was determined as indicated at 25° C. F-G. Coimmunoprecipitation (co-IP) of CypD and Aβ in brain mitochondria from AD patients and transgenic mice. Representative co-IP results demonstrated the presence of CypD-Aβ complex in the temporal cortex of AD patients (F1, lanes 5-7)) and in the cerebral cortex of Tg mAPP mice at 12-months-old age (G1, lanes 4-5). CypD-Aβ complex disappeared when anti-Aβ IgG was replaced by preimmune IgG (lane 8). Lane 4 in FIG. F1: Immunoblot for Aβ40 peptide (50 ng). (F2, G2). Quantification of the intensities of the immunoreactive bands generated from co-IP results using NIH image program (AD: n=9; age-matched ND: n=6; n=4-6 for Tg mAPP mice and nonTg littermates). * P<0.001 compared to ND or other groups of mice. H-I. Confocal microscopy shows staining of Aβ (red) and CypD (green) in the cortex of AD brain (H) and hippocampus of Tg mAPP mice at 12-month-old age (I). Colocalization of CypD-Aβ was demonstrated as the overlay images (yellow). Magnification ×200. (J-L). Electron microscopy with the double immunogold staining of CypD (12 nm gold particle) and Aβ (18 nm gold particle) demonstrated colocalization of CypD and Aβ in mitochondria of the brains from AD patients (J), age-matched ND control (K) and Tg mAPP mice (L).

FIG. 3. Deficiency of CypD protects against Aβ-mediated mitochondrial dysfunction in Tg mAPP mice. (A-B). Swelling in brain mitochondria isolated from the cortex of the indicated Tg mice in response to the Pi (1 μM)). Data are shown as changes of percentage (A1-3) and rate/min (B 1-3) in mitochondria induced by Pi relative to the vehicle-treated mitochondria. C1-2. Confocal microscopy of TMRM in brain slices of the indicated Tg mice. The intensity of TMRM staining in temporal cortex (C1) and hippocampus (C2) was quantified. * P<0.05 compared to the other groups of mice. N=4-6 mice/per group. D. Quantification of intensity of immunoreactive band for CypD in the inner mitochondrial membrane from the indicated Tg mice (n=3 mice/per group). * P<0.01 vs. Tg mAPP mice. (E). Representative Western blot for CypD as shown in panel D. F. Coimmunoprecipitation/immunoblotting for detection of CypD-Aβ complex in the mitochondrial inner membrane of the indicated Tg mice. The complex was disappeared when the preimmune IgG substituted for anti-CypD using in the immunoprecipitation (lane 1). G-I. Detection of ROS in brain mitochondria of the transgenic mice. Brain slices from the indicated Tg mice were stained with Mitosox and examined under confocal microscopy. The area occupied and intensity of Mitosox staining in temporal cortex and hippocampus of Tg mice were quantified by Universal image program (G-H) n=4-6 mice/per group, * P<0.01)). I. Representative confocal images for Mitosox staining in the indicated Tg mice. (J-K). Activity of cytochrome c oxidase (J) and ATP (K) were measured in the cerebral cortex of the indicated Tg mice at 12-month-old age (n=8-10 mice/per group, * P<0.05 vs. other groups of mice).

FIG. 4. Effect of deficiency of CypD in mitochondrial function induced by Aβ in vitro. (A) Coimmunoprecipitation of CypD and Aβ was performed in the isolated mitochondrial inner membrane of the cortex from nonTg and CypD null mice (CypD−/−) in the presence (+) or absence (−) of Aβ42 (0.8 μM) at 4° C. for 15 min. Aβ40 peptide (1 μg) in lane 6 serves as a positive control for immunoblotting of Aβ. (B) Immunoblotting for CypD in the isolated inner membrane of the cortical mitochondria treated with Aβ42 (0.8 μM), Ca2+, Aβ plus CsA (1 μM), or Ca2+ plus CsA. Lower panels of FIG. A-B show immunoblotting for COX IV in mitochondrial fraction demonstrating an equal amount of mitochondrial protein applied to the experiment. (C) Swelling in isolated cortical mitochondria induced by Ca2+ (100 μM, red line), or in the presence of CsA (1 μM, blue line). (D) Comparison of calcium-induced swelling in the cortical mitochondria isolated from CypD-deficient mice and nonTg littermate controls. (E). Swelling induced by Aβ (0.8 or 1.6 μM) in cortical mitochondria derived from CypD-deficient mice and nonTg littermates. Addition of CsA (1 mM) to the mitochondrial reactions significantly suppressed Aβ-induced swelling (F). (G-I) Cytochrome c release induced by Aβ and by H2O2. Isolated nonTg and CypD-deficient cortical mitochondria were incubated with Aβ (2 μM, G-H), or H2O2 (500 μM, I) and the resultant supernatant and pellet were subjected to immunoblotting for cytochrome c and COX IV (control). (H). Quantification of intensity of immunoreactive band for cytochrome c release in the supernatant (n=3-5).

FIGS. 5. Aβ- and H2O2-induced mitochondrial and neuronal dysfunction in cultured neuron. (A) Western blot of cultured cortical neuron derived from nonTg and CypD-deficient mice was performed by specific α-CypD IgG. (B) Immunoprecipitation of primary cultured cortical neuron derived from nonTg and CypD-deficient mice with α-CypD IgG followed by immunoblot with α-Aβ IgG (6E10) showed the presence of CypD-Aβ complex in nonTg-derived neurons (lane 2), not in CypD-deficient neurons (lane 4) exposed to Aβ for 24 hours. C-D. Fluorescence intensity of TMRM in cultured neurons treated with oligomeric Aβ42 at the indicated time (C, 5 μM) and various doses of Aβ for 24 hours treatment (D). *P<0.01 vs. CypD-derived neurons treated with Aβ. As a positive control, addition of FCCP (1 μM), was able to dissipate mitochondria ΔΨ in both nonTg and CypD−/− neurons, #P<0.001 vs. other groups of neurons. (E) Percentage of TUNEL positive neuron in cultured cortical neurons derived from nonTg and CypD−/− mice exposed to Aβ42 (5 μM), or in the presence of CsA (1 μM) for the indicated time. (F-G) FACS analysis of TMRM staining in nonTg and CypD−/− neurons treated with increasing concentrations of H2O2 for one hour. (G) Analyses of % TMRM positive neurons combined 3-4 independent experiments. FCCP serves as a control for mitochondrial uncoupler. *P<0.01 and # P<0.001 compared to other groups of neurons. (H-I) TMRM staining in H2O2-treated nonTg and CypD−/− neurons as shown by confocal microscopy in live cells. (J-L). FACS analysis of propidium iodide (PI) staining in nonTg and CypD−/−neurons treated with H2O2 for one hour. Percentages of PI-positive and -negative cells were shown as red line and black line, respectively. (O-M) FACS analysis of Annexin V-FITC staining in nonTg and CypD−/− neurons treated with the indicated concentrations of H2O2. for one hour. *P<0.001 vs. nonTg-derived neurons treated with H2O2 and vehicle-treated neurons. #P<0.01 in FCCP-treated (1 μM) neurons compared to vehicle-treated neurons.

FIG. 6. Spatial learning and memory and AChE activity in Tg mice: effect of absence of CypD. A-B. Radial water maze test for the spatial learning and memory in the indicated Tg mice at 6 or 12 month-old age. C. AChE activity in hippocampus of indicated Tg mice. *P<0.05 vs. other groups of mice (n=8-10 mice/per group).

FIG. 7. Expression of CypD in the cerebral cortex of Parkinson disease (PD) and age-matched/nondemented controls (ND). Western blot of brain homogeneous with specific antibody for human CypD showed an increased levels of CypD in the brains of Parkinson disease (PD) patients compared to ND.

FIG. 8 Effect of CypD deletion on spatial learning and memory in Tg mice and Aβ-induced LTP. FIG. 8A. Slices from 12-13 month old mAPP mice showed a reduction of LTP compared to nonTg slices [F(1,20)=4.855, P=0.0256]. In contrast, depletion of CypD in Tg mAPP mice (Tg mAPP/CypD−/−) displayed normal LTP [F(1,21)=4.855, P=0.0389]. LTP was normal in slices from CypD−/− littermates [F(1,20)=0.7049 P=0.4111]. FIG. 8B. Depletion of CypD in slices from knock-out mice protected them against reduction of LTP by perfusion with 200 nM Aβ42 for 20 min through the bath solution, in contrast to slices from nonTg mice perfused with the peptide, which showed a reduction of LTP [F(1,14)=7.760, P=0.0146]. LTP was normal in slices from vehicle-treated CypD−/− mice compared to vehicle-treated nonTg littermates [F(1,11)=0.003482 P=0.9540]. Basal synaptic transmission was not affected in the CypD−/− mice. The horizontal bar indicates the period during which Aβ42 was added to the bath solution in this and the other graphs. The arrows indicate the tetanus application in this and the other graphs. FIG. 8C. The CypD inhibitor CsA prevented Aβ-induced inhibition of LTP in nonTg hippocampal slices [F(1,13)=6.188, P=0.027 vs. Aβ-treated nonTg slices]. CsA alone did not alter LTP [F(1,11)=0.0003, P=0.9857 vs. vehicle-treated nonTg slices]. 8D. Scavenging superoxide through perfusion with superoxide dismutase (SOD) plus catalase prevented Aβ-induced inhibition of LTP in nonTg hippocampal slices [F(1,13)=5.088, P=0.042 vs. Aβ-treated nonTg slices]. SOD and catalase alone did not alter LTP [F(1,12)=0.0038, P=0.925 vs. vehicle-treated nonTg slices].

FIG. 9 Effect of CypD deficiency on basal synaptic transmission (BST). Slices from 12-13 month old mAPP mice showed a reduction of BST compared to nonTg slices ([F(1,27)=11.01, P=0.0026]. In contrast, depletion of CypD in Tg mAPP mice (Tg mAPP/CypD−/−) protected slices from APP littermates against reduction of BST [F(1,30)=5.159, P=0.0305]. BST was normal in slices from CypD−/− littermates [F(1,20)=0.5476 P=0.4678].

### DEFINITIONS

A subject at risk of developing AD or PD is a subject which is not officially diagnosed with the disease but shows a symptom of the disease, is susceptible to it due to family history or genetic predisposition, or has CypD levels in a biological sample (preferably blood, serum, or csf) that are significantly higher than normal.

A therapeutically effective amount of a protein or polypeptide (i.e., an effective dosage) or nucleic acid (such as antisense nucleotides), is an amount that achieves the desired therapeutic result. For example, a therapeutically effective amount is an amount that ameliorates one or more symptoms of the disease, including AD and PD, or that reduces the expression of CypD in a subject or the level of CypD in a biological sample from the subject that has or is at risk of developing AD or PD, or that reduces the ability of CypD to form a complex with amyloid beta protein.

Significantly lower or significantly higher means that the difference is statistically significant. For example the post-treatment serum level of CypD is significantly lower than the pre-treatment level if the difference is statistically significant.

Cyclophylin D (CypD) means peptidylprolyl isomerase F (PPIF, cyclophilin F (mitochondrial form), Gene bank accession #BC005020, M80254, AAA58434, AAH05020.) and includes all forms thereof, including biologically active analogs, derivatives, fragments and variants. Cyclophilin D is found in the matrix and the inner membrane of mitochondria. Cyclophilin D is involved in mitochondrial permeability transition, in which the adenine nucleotide translocase of the inner membrane is transformed from an antiporter to a non-selective pore.

Cyclosporine includes all forms including biologically active analogues, derivatives, synthetic forms, isolated and purified forms, recombinant forms and biologically active fragments or variants thereof. Initially isolated from a Norwegian soil sample, Cyclosporin A, the main form of the drug, is a cyclic nonribosomal peptide of 11 amino acids (an undecapeptide) produced by the fungus Tolypocladium inflatum Gams, and contains D-amino acids, which are rarely encountered in nature. Cyclosporin A, a preferred embodiment for treating or preventing AD or PD, blocks the formation of the mitochondrial permeability transition pore. Additional cyclosporine analogues are disclosed in WO 99/18120. The terms Ciclosporin, ciclosporin, cyclosporine, and Cyclosporine are interchangeable and refer to cyclosporine. Certain other analogues are described in U.S. Pat No. 7,332,472 Naicker, et al. The derivative is NIM811 is one which specifically inhibits MPT pore opening. Cyclosporine is a potent immunosuppressive agent that suppresses humoral immunity and cell-mediated immune reactions such as allograft rejection, delayed hypersensitivity, experimental allergic encephalomyelitis, Freund's adjuvant arthritis and graft vs. host disease. Since the original discovery of cyclosporine, a wide variety of naturally occurring cyclosporines have been isolated and identified and many further non-natural cyclosporines have been prepared by total- or semi-synthetic means or by the application of modified culture techniques. The class comprised by the cyclosporines is thus now substantial and includes, for example, the naturally occurring cyclosporines A through Z. Various non-natural cyclosporine derivatives and artificial or synthetic cyclosporines include the dihydro- and iso-cyclosporines; derivatized cyclosporines (e.g., in which the 3′-O-atom of the -MeBmt-residue is acylated or a further substituent is introduced at the alpha-carbon atom of the sarcosyl residue at the 3-position); cyclosporines in which the -MeBmt-residue is present in isomeric form (e.g., in which the configuration across positions 6′ and 7′ of the -MeBmt-residue is cis rather than trans); and cyclosporines wherein variant amino acids are incorporated at specific positions within the peptide sequence as described in U.S. Pat. Nos. 4,108,985, 4,210,581, 4,220,641, 4,288,431, 4,554,351 and 4,396,542; European Patent Publications Nos. 0 034 567 and 0 056 782; International Patent Publication No. WO 86/02080; incorporated herein by reference. Immunosuppressive, anti-inflammatory, and anti-parasitic cyclosporine A analogues are described in U.S. Pat. Nos. 4,384,996; 4,771,122; 5,284,826; and 5,525,590, all assigned to Sandoz. Cyclosporin is marketed by Novartis under the brand names Sandimmune™, the original formulation, and Neoral™ for the newer microemulsion formulation. Generic ciclosporin preparations have been marketed under various trade names including Cicloral™ (Sandoz/Hexal) and Gengraf™ (Abbott). The drug is also available in a dog preparation manufactured by Novartis called Atopica™. The above references are incorporated by reference as if set forth herein in their entirety.

The terms “inhibit”, “elevate”, “increase”, “decrease” or the like, e.g., which denote quantitative differences between two states, refer to a difference, e.g., a statistically significant difference, between the two states

Transgenic animals means animals that carry a segment of foreign DNA that has been incorporated into their genome via non-homologous recombination (e.g., pronuclear microinjection), insertion via infection with a retroviral vector, or in some cases, by homologous insertion. Examples of transgenic animals include rodents, preferably mice, non-human primates, sheep, dogs, cows, goats, chickens, and amphibians.

Knock Out mice means mice with targeted mutations that are created by first introducing either gene disruptions, replacements, or duplications into embryonic stem (ES) cells by homologous recombination between the exogenous (heterogeneous or targeting) DNA and the endogenous (target) gene. The mutation in the nucleic acid sequence of the gene reduces the biological activity of the polypeptide normally encoded by the gene. When one allele is knocked out (+/−) typically by about 50% of the biological activity is lost compared to the unaltered gene. When both alleles is knocked out (−/−) typically by about 100% of the biological activity is lost compared to the unaltered gene. The alteration may be an insertion, deletion, frame shift mutation, or missense mutation. The genetically-modified ES cells are then microinjected into host embryos at the 8-cell blastocyst stage. These embryos are transferred into pseudo pregnant host females, which then bear chimeric progeny. The chimeric progeny that carry the targeted mutation (i.e., the “knocked out” gene) in their germ line are then bred to establish the “knockout” line.

### DETAILED DESCRIPTION

Mitochondria are central players in mediating neuronal stress relevant to the pathogenesis of Alzheimer's disease (AD). The mitochondrial permeability transition causes mitochondrial swelling, outer membrane rupture, release of cell death mediators and enhances production of reactive oxygen species (ROS). Cyclophilin D, a prolyl isomerase located within mitochondrial matrix, is an integral part in the formation of the mitochondrial permeability transition pore (mPTP), leading to cell death. Until now, the role of Cyclophilin D in Alzheimer disease has not been elucidated. We have now discovered that cyclophilin D interacts with amyloid beta peptide (Aβ) within the mitochondria of AD patients to form a complex that is responsible for much of the damage to mitochondria in AD cells. Mitochondria isolated from Alzheimer disease mice lacking cyclophilin D are resistant to Aβ- and Ca2+-induced mitochondria swelling and permeability transition, increased calcium buffering capacity, and attenuated generation of mitochondrial ROS. Furthermore, CypD-deficient neurons protect against Aβ- and oxidative stress-induced cell death. Importantly, we found that a deficiency of Cyclophilin D greatly improved the learning, memory, and synaptic function of an AD-mouse model and alleviated Aβ-mediated reduction of long term potentiation. The cyclophilin D/Aβ complex-mediated mitochondrial permeability transition pore is therefore directly linked to the cellular and synaptic perturbation relevant to the pathogenesis of Alzheimer disease.

Thus certain embodiments of the present invention are directed to methods for treating or preventing AD by blocking Cyclophilin D expression or its ability to form a complex with Aβ. Antisense nucleotides and small interfering RNA that hybridize with the gene or mRNA for CypD can be administered therapeutically to reduce CypD expression in a subject having or at risk of developing AD. Cyclosporine (preferably cyclosporine A or D) can be used to inhibit CypD as a therapy, and anti-CypD antibodies can be administered in therapeutic amounts to inactivate CypD or prevent free CypD from complexing with Aβ, thereby preventing the CypD/Aβ complex from initiating a cascade of reactions that lead to mitochondrial toxicity and apoptosis of the neurons. These methods can be used to treat or prevent any disease associated with elevated CypD expression. We have discovered the amino acid sequence (SEQ ID NO. 3)of CypD (amino acid 97-119,R V I P S F M C Q A G D F T N H N G TG GK S) that encodes the region that binds to Aβ; therefore preferred antibodies for therapeutic use specifically bind to epitopes that include all or part of this region of CypD. Certain other embodiments are directed to CypD fragments that include all or part of this binding region, and to antibodies that specifically bind to this epitope.

Certain other embodiments are directed to methods for preventing damage caused by oxidative stress, or to reducing memory loss associated with aging, AD or PD, using similar therapies to reduce CypD expression, inhibit CypD activity, or inhibit it from complexing with amyloid beta.

### Background

Mitochondria are the main energy source in cells of higher organisms, and provide direct and indirect biochemical regulation of a wide array of cellular respiratory, oxidative and metabolic processes. Mitochondria have an outer mitochondrial membrane that serves as an interface between the organelle and the cytosol, a highly folded inner mitochondrial membrane that appears to form attachments to the outer membrane at multiple sites, and an intermembrane space between the two mitochondrial membranes. The subcompartment within the inner mitochondrial membrane is commonly referred to as the mitochondrial matrix. (For a review, see, e.g., Emster et al., 1981, J. Cell Biol. 91:227s.)

The mitochondrial permeability transition “pore” to any mitochondrial molecular component (including, e.g., a mitochondrial membrane per se) regulates the inner membrane selective permeability where such regulated function is impaired during MPT.

It is unresolved whether this pore is a physically discrete conduit that is formed in mitochondrial membranes, for example by assembly or aggregation of particular mitochondrial and/or cytosolic proteins and possibly other molecular species, or whether the opening of the “pore” may simply represent a general increase in the porosity of the mitochondrial membrane.

A hallmark pathology of AD and potentially other diseases associated with altered mitochondrial function is the death of selected cellular populations in particular affected tissues, which results from apoptosis (also referred to as “programmed cell death” or PCD). Mitochondrial dysfunction is thought to be critical in the cascade of events leading to apoptosis in various cell types (Kroemer et al., FASEB J. 9:1277-87, 1995), and may be a cause of apoptotic cell death in neurons of the AD brain. Altered mitochondrial physiology may be among the earliest events in PCD (Zamzami et al., J. Exp. Med. 182:367-77, 1995; Zamzami et al., J. Exp. Med. 181:1661-72, 1995) and elevated reactive oxygen species (ROS) levels that result from such altered mitochondrial function may initiate the apoptotic cascade (Ausserer et al., Mol. Cell. Biol. 14:5032-42, 1994).

The cyclophilins (Cyps) are a family of ubiquitous proteins expressed in all organisms. All Cyp family members share a conserved core of about 109 amino acids, but differ from one another by unique extensions that function in organelle and membrane transport (e.g., Walsh et al., 1992 J. Biol. Chem. 267:13115-18). At least eight human Cyp isoforms are known, including single domain and two-domain cyclophilins (e.g., Taylor et al., 1997 Prog. Biophys. Mol. Biol. 67:155-81, which reference is incorporated herein by reference. Distinct isoforms localize to different cell compartments, including cytoplasmic, endoplasmic reticulum (ER), mitochondrial, and cell surface isoforms (Handler et al. EMBO J. 6: 947-50, 1987; Price et al. Proc. Natl. Acad. Sci. USA 88: 1903-07, 1991; Bergsma et al. J. Biol. Chem. 266: 23204-14; Cacalano et al. Proc Natl Acad Sci USA 89: 4353-57, 1992).

Cyclophilins are believed to perform multiple functions within cells. For example, they catalyze the interconversion of cis and trans isomers of peptidylprolyl bonds in peptides and proteins, thereby facilitating the folding of proteins for which isomerization of peptidylprolyl bonds is rate limiting (see, e.g., Galat, Eur. J. Biochem. 216:689-707, 1993; Fischer et al., Biochem. 29:2205-2212, 1990; Stamnes et al., Cell 65:219-27, 1991). This peptidylproyl cis-trans-isomerase activity can be blocked by the immunosuppressant cyclosporin A (e.g., Fruman et al., Proc. Natl. Acad. Sci. USA 89:3741-45, 1992). Cyp family members also appear to mediate other activities by forming complexes with fully folded, functional proteins (see, e.g., Jaschke et al., J. Mol. Biol. 277:763-69, 1998; Ratajczk et al., J. Biol. Chem. 268:13187-92, 1993; Wu et al., J. Biol. Chem. 270:14209-19, 1995; Holloway et al., J. Biol. Chem. 273:16346-50, 1998; Franke et al., Adv. Exp. Med. Biol. 374: 217-28, 1995).

CypD is the only mitochondrial isoform of the Cyp family identified to date. It is also referred to in the literature as CypF, which is peptidylprolyl isomerase F (PPIF, cyclophilin F (mitochondrial form). Cyclophilin D has Gene bank accession #BC005020, M80254, AAA58434, AAH05020). The human CypD polypeptide is 207 amino acids long and has an amino-terminal hydrophobic extension, which may serve to transport the polypeptide across mitochondrial membranes to the matrix (Bergsma et al., J. Biol. Chem. 266:23204-14, 1991). Both mouse and human CypD have the same DNA sequences. This 100% sequence homology between mouse and human makes our results in mice predictive of human results.

Cyp D is believed to participate in the formation of the mitochondrial permeability transition pore by interacting with the voltage-dependent anion channel (VDAC) and with ANT, at contact sites between the mitochondrial outer and inner membranes (Crompton et al., Eur. J. Biochem. 258 729-35, 1998; Woodfield et al., 1998, Biochem. J. 336:287-90). CypD binding to ANT may also sensitize the pore complex to calcium concentration (Halestrap et al., Biochim. Biophys. Acta. 1366:79-94, 1998). This opening of the mitochondrial permeability transition pore has been suggested to be an event in the pathogenesis of diseases associated with altered mitochondrial function.

CypD translocation from the matrix to the inner membrane is a key factor for triggering the formation of mPTP. Oxidative and other cellular stresses induce CypD translocation to the inner membrane, where it binds to the adenine nucleotide translocase (ANT) to form the mPTP. Opening of the mPTP collapses the membrane potential and amplifies apoptotic mechanisms by releasing proteins with apoptogenic potential from inner membrane space [12-14]. Release of CypD from the matrix allows it to bind to adenine nucleotide translocase (ANT), and potentially to other targets on the inner mitochondrial membrane, which interaction contributes to opening the mPTP that in turn leads to necrosis and apoptosis.

### Expression of CypD is Elevated in the Human AD Brain and in Brains of Transgenic APP Mice

To assess the significance of CypD in Alzheimer disease, we examined the expression levels of CypD in brains from AD patients and age-matched, non-demented (ND) controls. Two AD affected brain regions were analyzed: temporal gyms and hippocampus. Mitochondria were isolated from the cortex of these two brain regions and subjected to immunoblotting with specific polyclonal anti-human CypD antibody (generated in our laboratory) that was prepared by immunizing a rabbit with full length human CypD protein. Since CypD is located inside the cell, anti-CypD is preferably bound to TAT peptide to facilitate its entry into the neuron where it can bind to and CypD and prevent it from forming a complex with amyloid beta. Details are described in Example 1

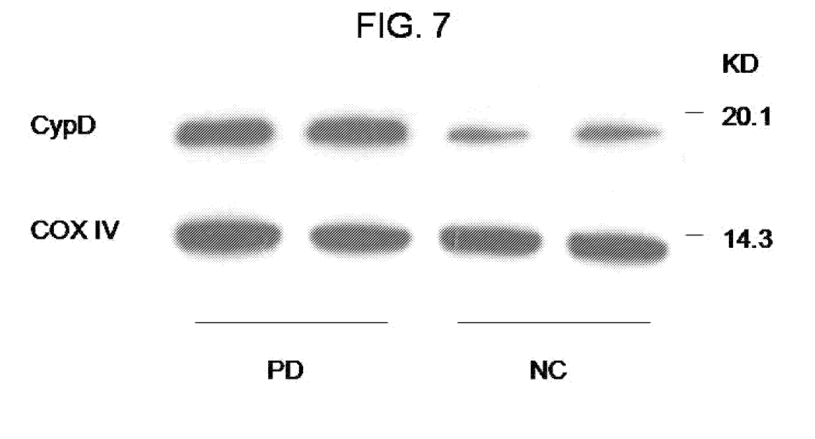

We have discovered that the region of CypD polypeptide from amino acid 97-110 (R V IP S F M C Q A G D F T N H N G T G G K S) is the region that binds to Aβ forming the CypD/Aβ complex. Antibodies that bind to all or part of this region of CypD are preferred for therapy of AD by blocking formation of the CypD/Aβ complex. As is shown in FIG. 7 expression of CypD is also elevated in the cerebral cortex of Parkinson disease (PD) and age-matched/nondemented controls (ND).

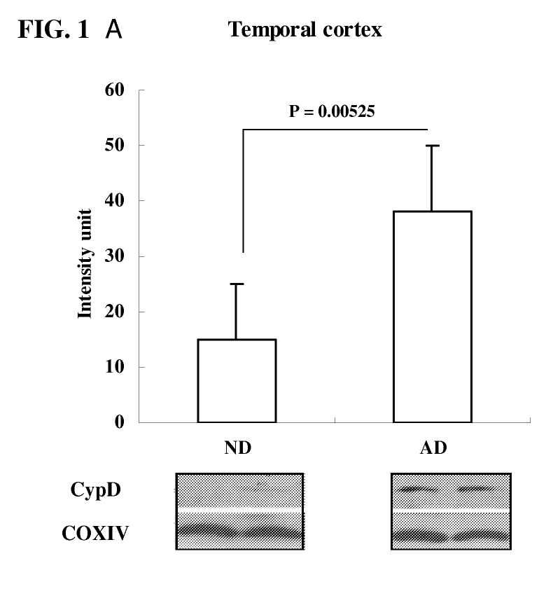

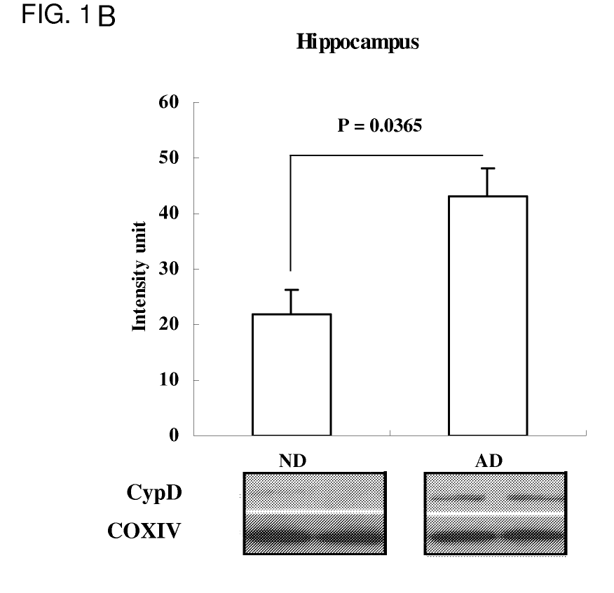

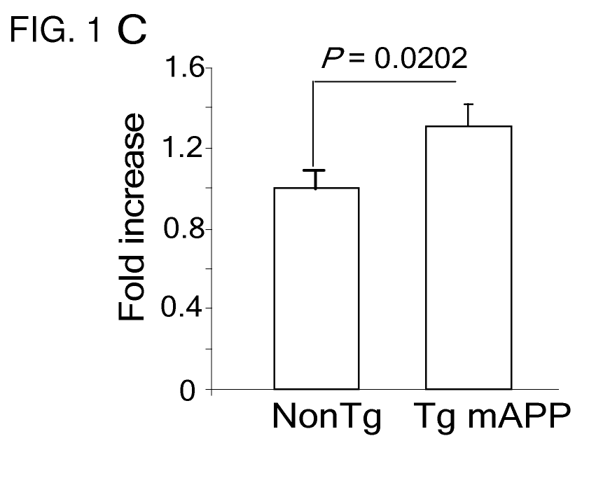

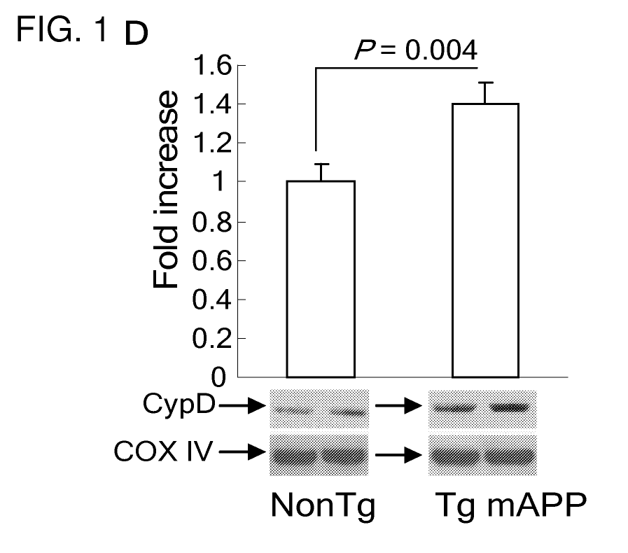

We discovered that there is a significant increase in the amount of CypD protein in the AD-affected regions in AD patients (˜60% in temporal cortex and ˜40% in hippocampus) versus age-matched, non-demented (ND) controls (FIG. 1A-B). In contrast, protein extracts prepared from the cerebellum, a region spared in AD, showed no significant differences between AD patients and ND controls (data not shown). Immunostaining of the temporal cortex and hippocampus of AD patients likewise showed a similar upregulation of CypD antigen (not shown). Increased expression of CypD was predominately localized to neurons (data not shown).

Based on this and other observations described herein, certain embodiments of the invention are directed to a method for diagnosing a human patient at risk of developing Alzheimer's disease by a.) determining a patient level of cyclophilin D, or a biologically active analog, derivative, variant or fragment thereof in a biological sample taken from the patient and a control level of cyclophilin D in a biological sample taken from a subject that does not have Alzheimer's Disease, b.) comparing the patient and control levels, and c.) concluding that the patient is at risk of developing Alzheimer's Disease if the patient level is significantly higher than the control level. The biological sample is preferably csf, serum, plasma, blood, neuronal tissue, or fibroblasts. We will show that or Parkinson disease patients also show elevated CypD levels in the brain, therefore this diagnostic method applies to or Parkinson disease also.

Consistent with the observations of human AD brain, transgenic (Tg) mice expressing a mutant form of human amyloid precursor protein (Tg mAPP mice) that encodes hAPP695, hAPP751, and hAPP770 bearing mutations linked to familial AD (V717F, K670M, N671L) also displayed elevated levels of CypD m RNA and protein in the critical cerebral cortex and hippocampus regions compared with nonTg littermate controls. mRNA levels were measured by quantitative real-time PCR (FIG. 1C) and by immunoblotting with antibody to CypD (FIG. 1D). The transcript for CypD was significantly increased ˜30% in the cerebral cortex of Tg mAPP mice compared to nonTg littermates and CypD protein measured by immunoblotting with the specific antibody to CypD was also elevated by ˜30-40% (FIG. 1D). As expected, there was no significant difference in the level of CypD protein in the cerebellum of Tg mAPP mice compared to nonTg littermate controls. Increased expression of CypD was predominately present in neurons in the cortex and hippocampus of Tg mAPP mice as was shown by immunostaining with our polyclonal antibody to CypD (data not shown). These data indicate that CypD expression is significantly increased in an Aβ-rich environment, both in AD brain and in the brain of Tg mAPP mice, showing the significance of CypD in the pathogenesis of AD.

In further work we discovered that CypD is also significantly elevated in the cerebral cortex taken from patients having Parkinson's disease compared to controls having no disease (ND). FIG. 7 shows the expression levels of CypD in the cerebral cortex of Parkinson disease (PD) and age-matched/nondemented controls (ND), using Western blot of brain homogeneous with specific antibody for human CypD. Thus PD can be treated in the same ways described herein for treating AD.

Interaction of CypD with Aβ and Formation of CypD/Aβ Complex within Brain Mitochondria of AD Patient and Transgenic APP Mice

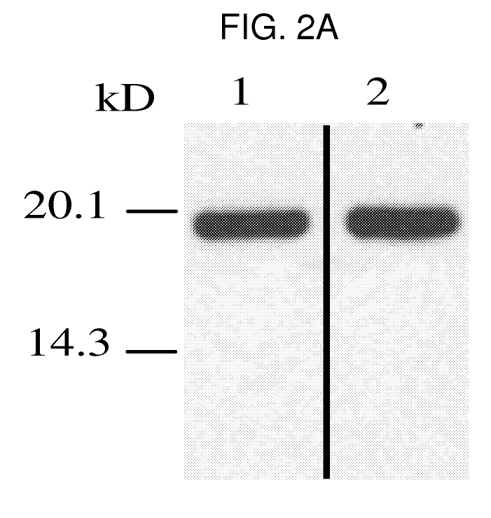

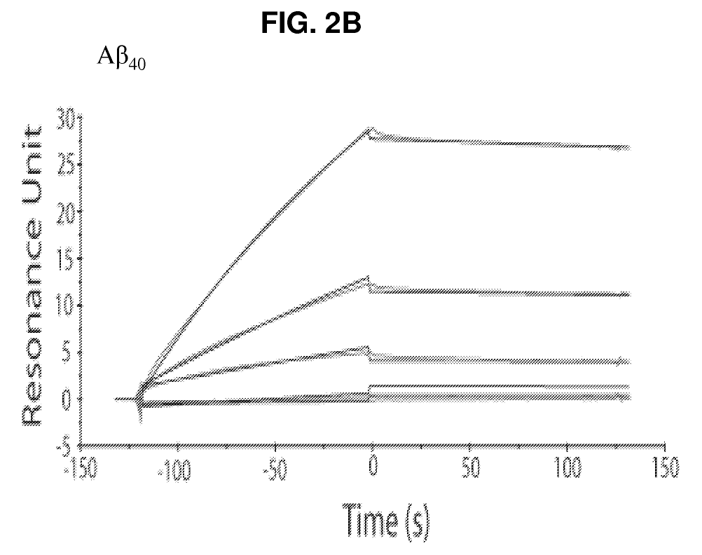

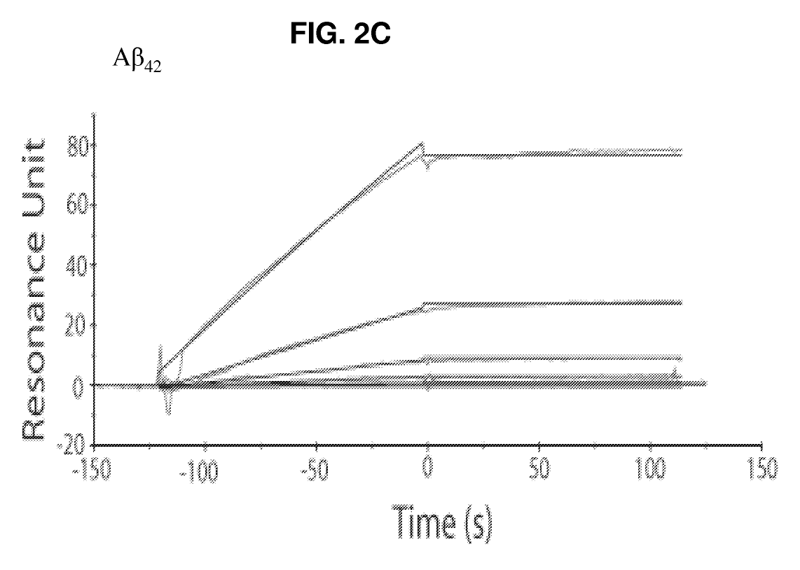

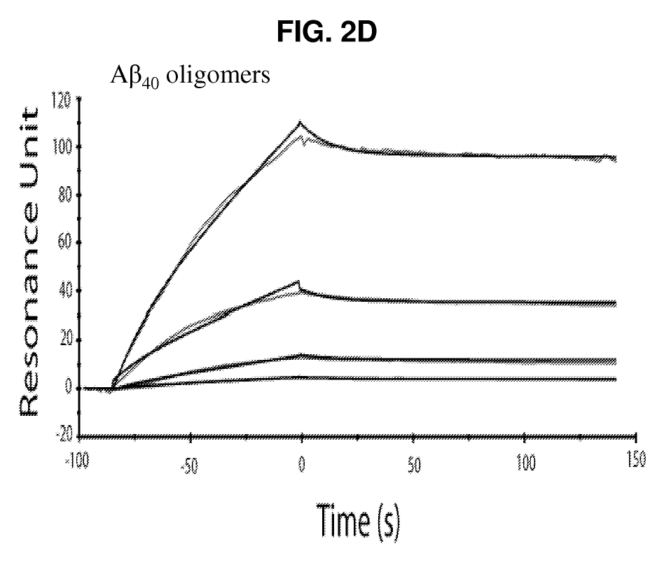

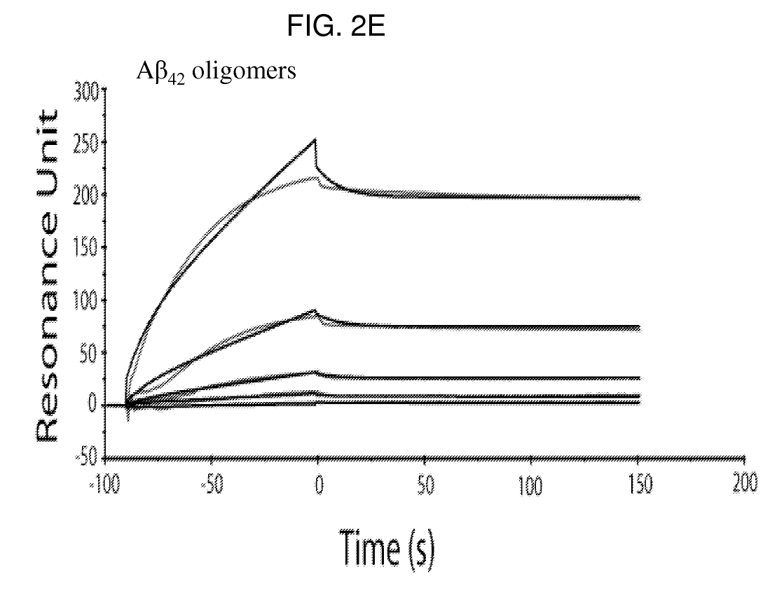

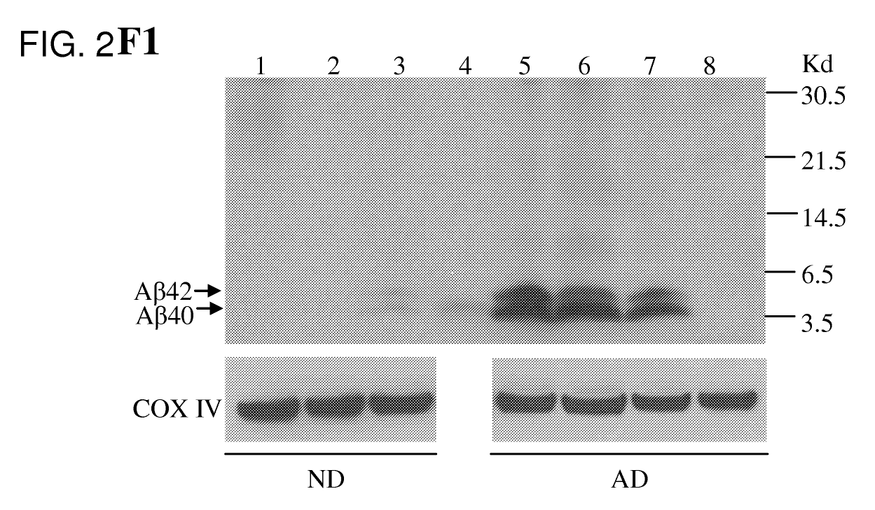

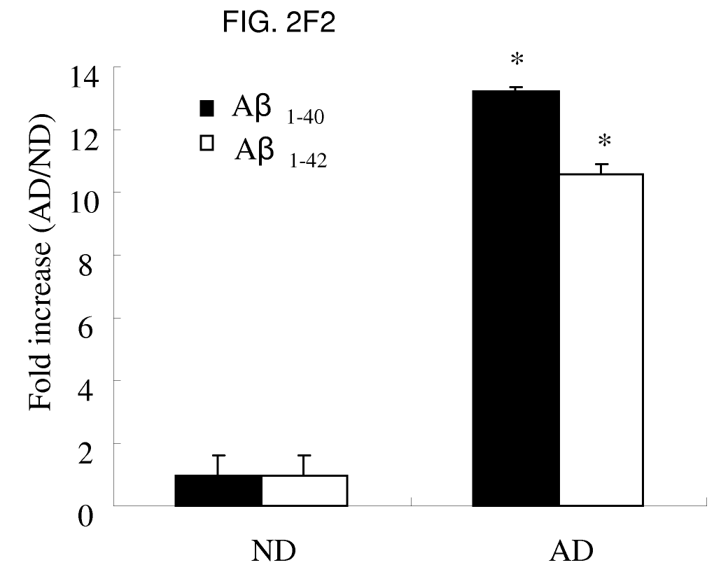

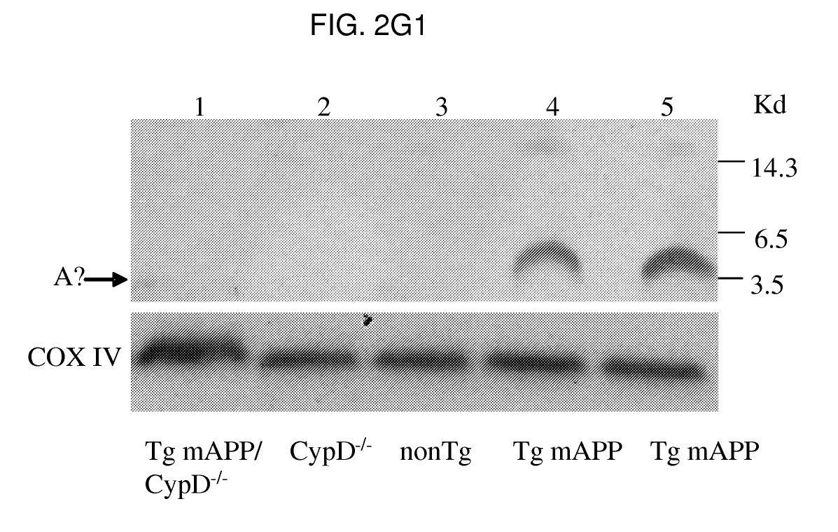

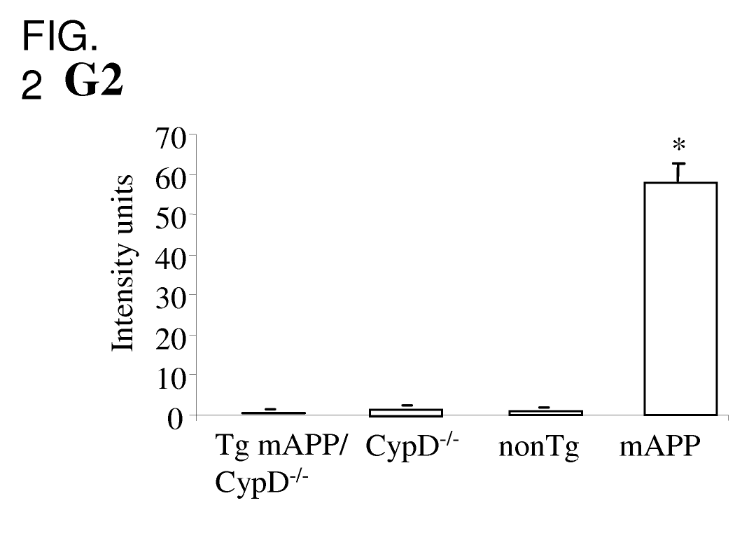

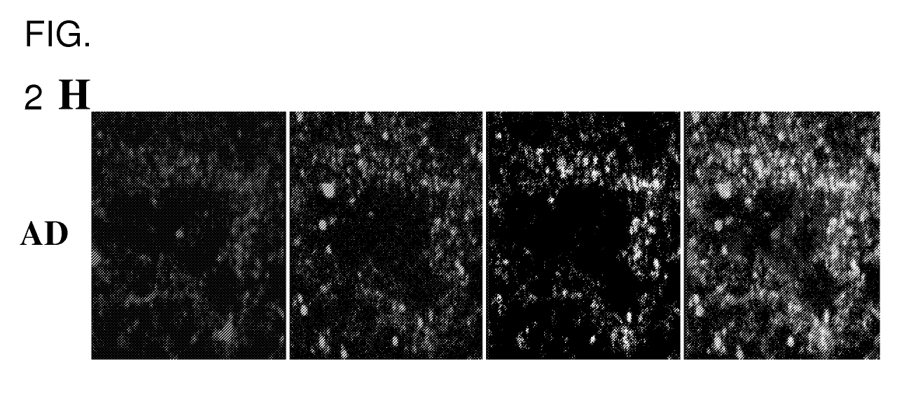

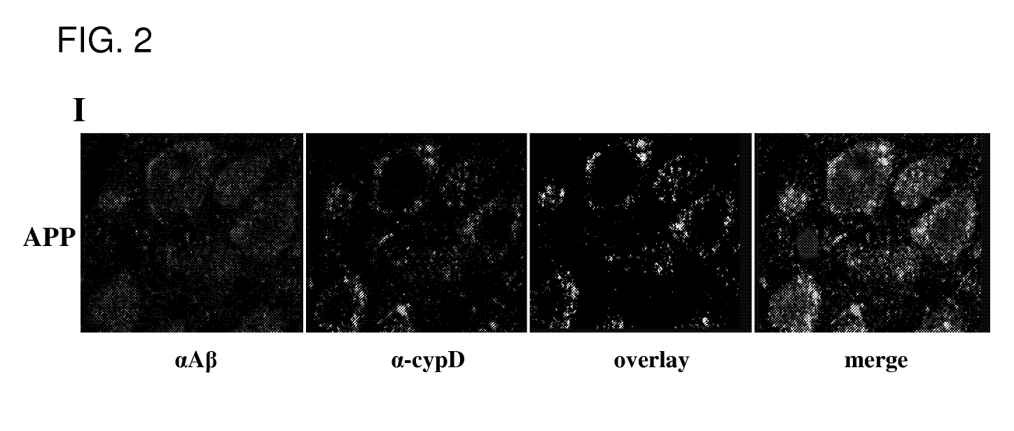

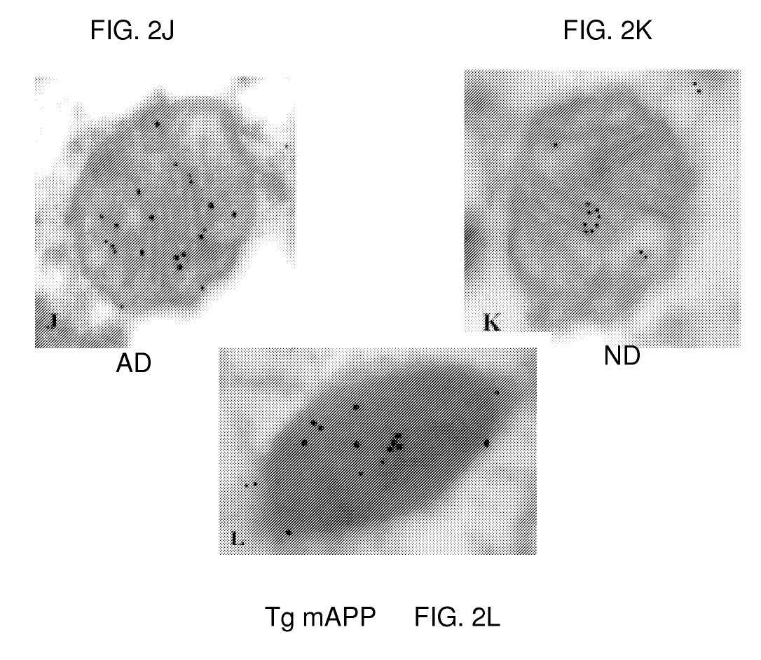

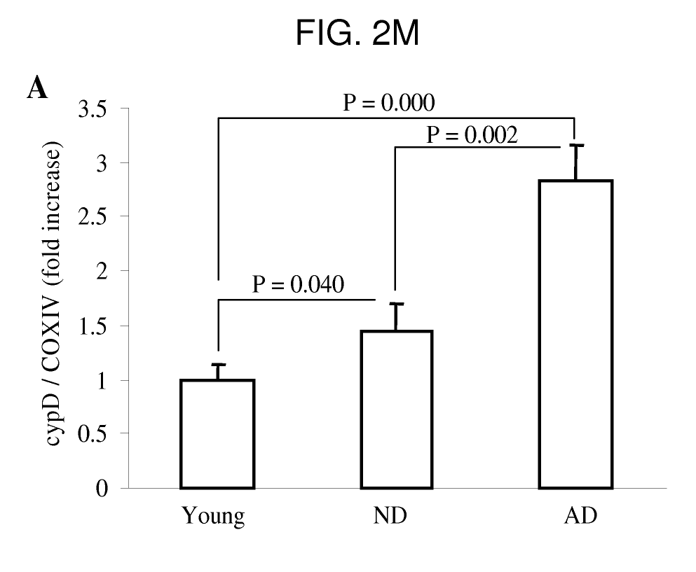

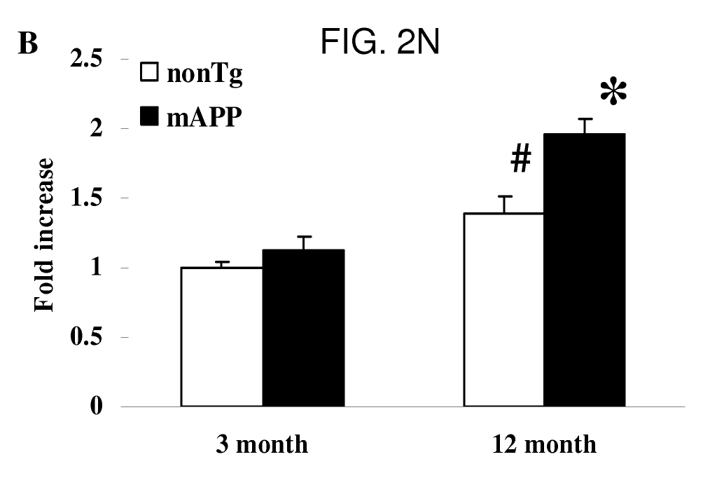

To study binding of CypD to Aβ, we expressed a GST-fusion protein in E. coli, cleaved with thrombin, and purified it to homogeneity. In view of CypD localization to key intracellular compartments in the mitochondria, it was essential to determine if CypD and Aβ actually interacted in the AD brain. The experiments described further in Example 2 revealed a greatly increased level of CypD-Aβ complex in cortical mitochondria from the AD brain compared to the cortical mitochondria from ND brain controls (FIG. 2F2).

Since cellular and mitochondrial integrity were likely to have deteriorated significantly soon after death and the accompanying tissue disruption might allow potentially nonphysiologic interactions to occur, we isolated mitochondria from cerebral cortex of Tg mice where sample quality was carefully controlled. To determine the specific interaction of CypD with Aβ in mitochondria, we also examined whether the CypD-Aβ complex was found in the cortical mitochondria from transgenic knock-out mice with a deficiency of CypD gene (Tg CypD−/−) and double Tg mice expressing mAPP and deletion of CypD gene (Tg mAPP/CypD−/−). Generation and characterization of transgenic mice are described in Example 3; the experiments on mitochondria from Tg mice are set forth in Example 4. We discovered that CypD forms a complex with Aβ in the brain mitochondria from both AD and transgenic APP mice that express elevated levels of APP. Colocalization of CypD and Aβ and their interaction in mitochondria of the cerebral cortex of AD patients was further confirmed by confocal and electron microscopy. FIG. 2 H-L. Colocalization of CypD with Aβ, at least in part, within mitochondria of both in AD brain and transgenic mice, is consistent with CypD-Aβ complex formation within mitochondria in vivo. These data indicate that all species of Aβ bind to CypD, and that oligomeric Aβ has a higher binding affinity than monomeric Aβ. Details are set forth in Example 2.

Deficiency of CypD and the Addition of Cyclosporin A Protects from Aβ-Induced Mitochondrial Dysfunction in Tg mAPP Mice

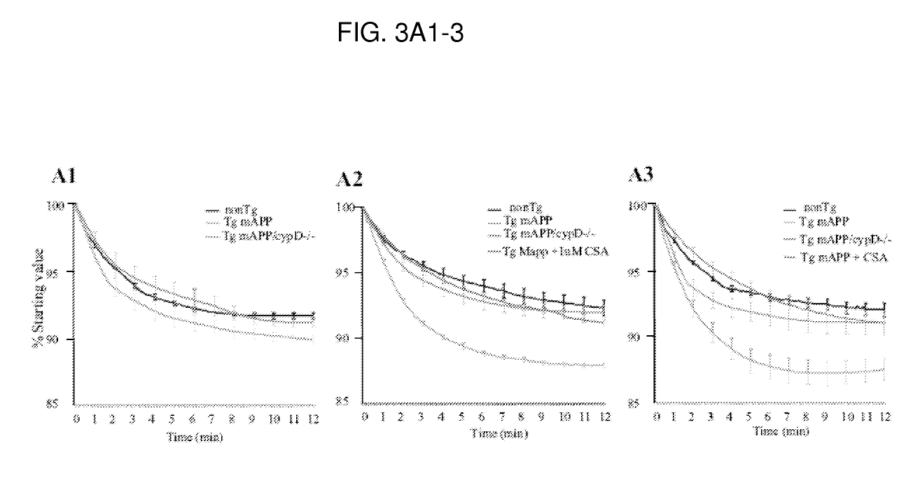

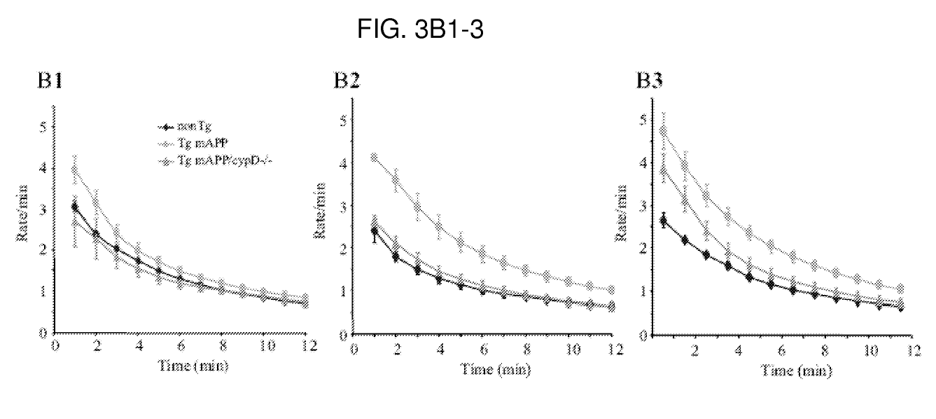

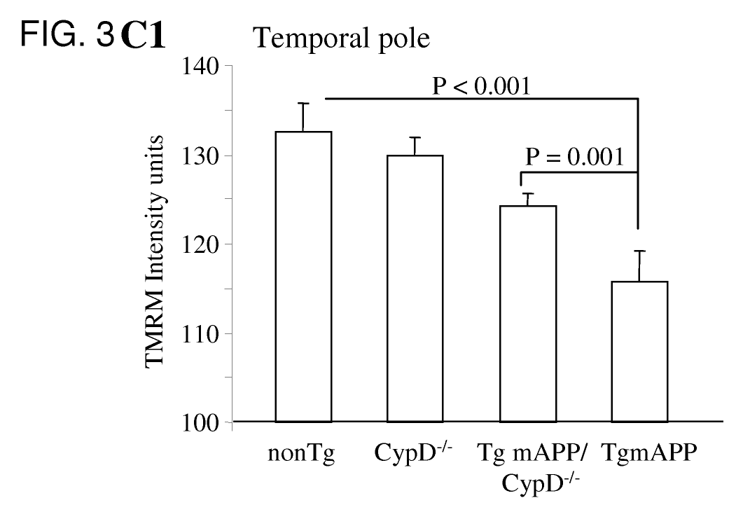

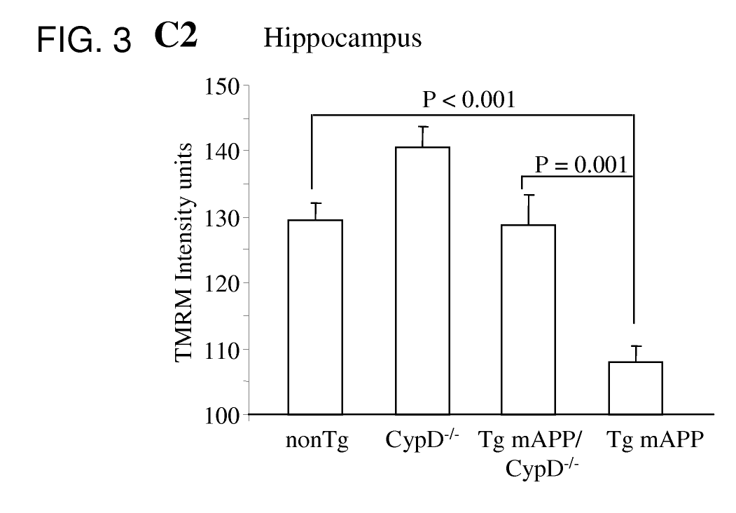

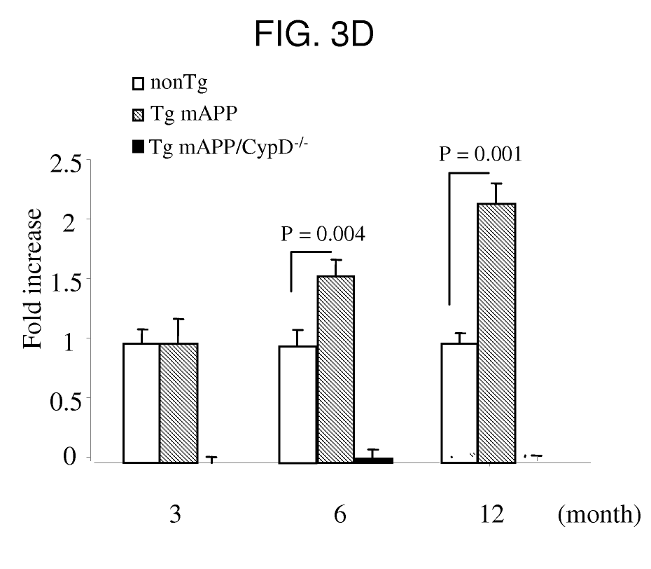

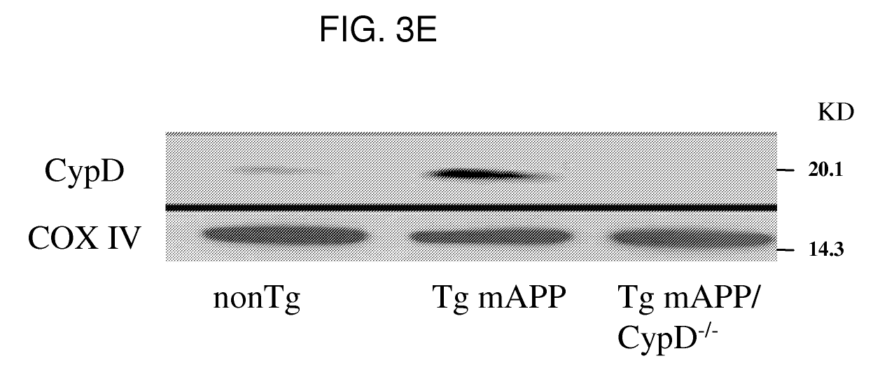

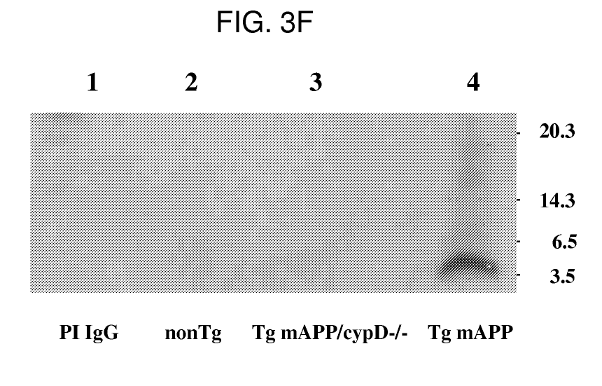

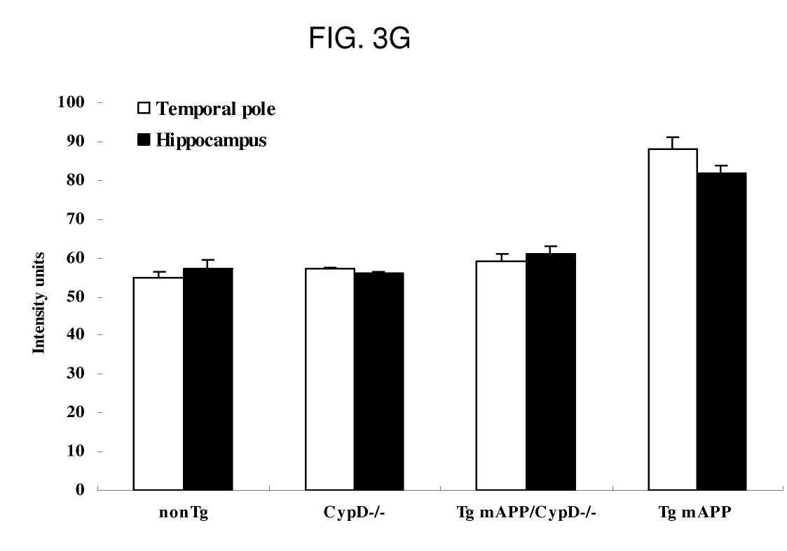

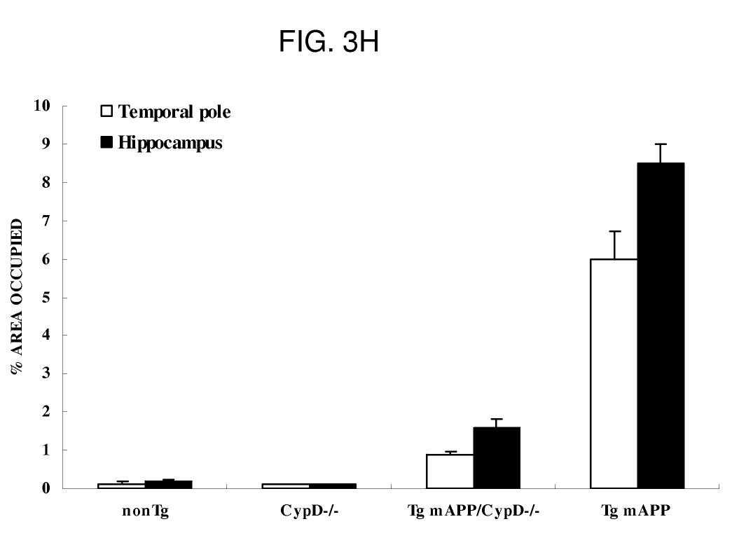

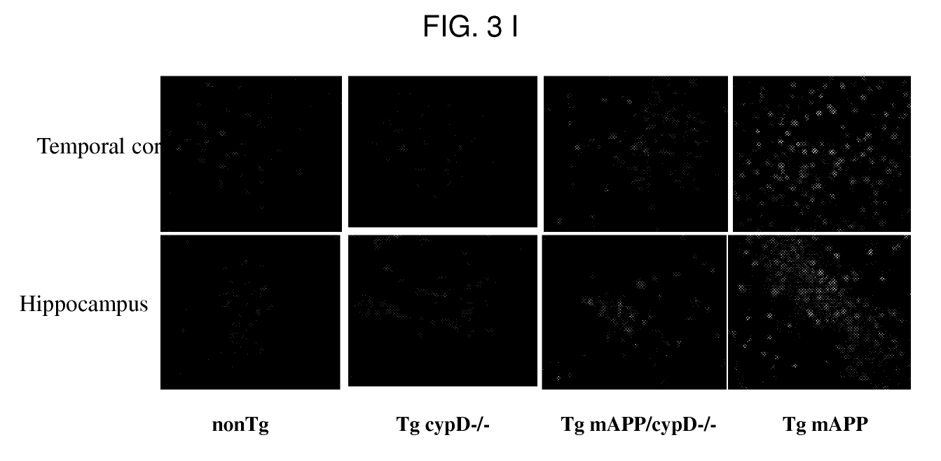

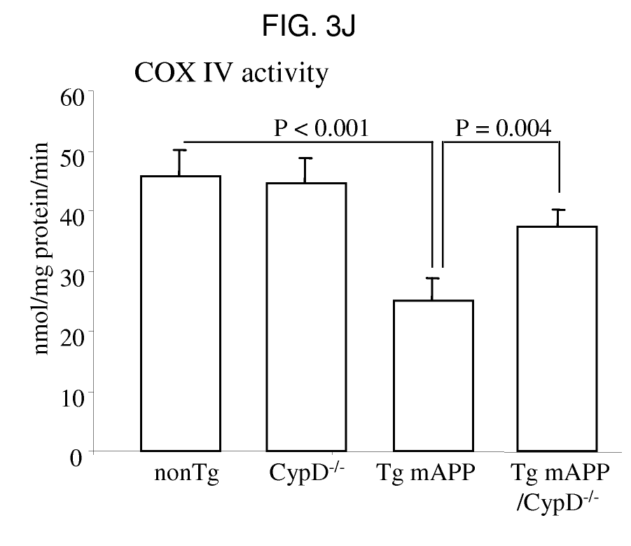

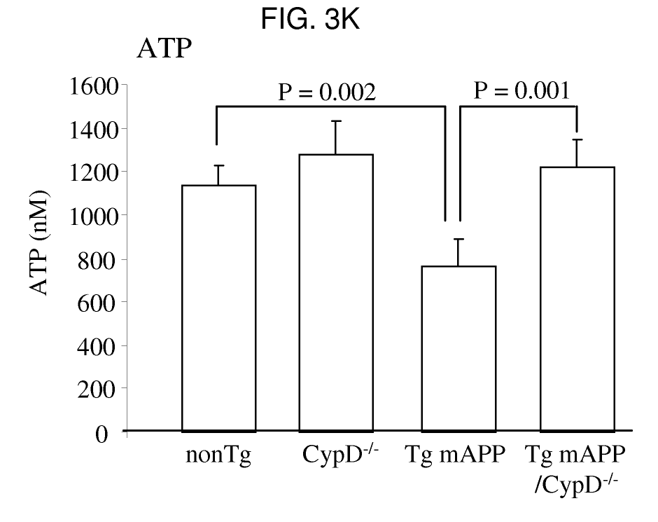

CypD serves to open the mitochondrial membrane thereby allowing the diffusion of solutes out of the mitochondria matrix to the cytoplasm where they cause cell death. To examine the role of CypD in the mitochondrial permeability transition (mPT) in the Aβ-rich environment, mitochondria were isolated from the cerebral cortex of knock-out mice Tg mAPP and Tg mAPP/CypD−/− mice and their nonTg littermate controls at different ages. Both strains of Tg mice showed age-dependent changes of swelling in response to phosphate (Pi), but they showed no significant changes in mPT induced by Pi among these three groups of mice at 3 months of age (FIG. 3A1). There was, however, a significant decrease in mPT in Tg mAPP mice compared to double mutant Tg mAPP/CypD−/− and non Tg littermates (FIG. 3A2-3) at 6 and 12 months of age, which is consistent with age-dependent accumulation of Aβ in mitochondria of Tg mAPP mice [8]. Importantly, mitochondria isolated from the cerebral cortex of Tg mAPP/CypD knock-out mice were resistant to swelling and permeability transition induced by Pi compared to mitochondria isolated from single Tg mAPP mice.

The fact that deletion of CypD caused mitochondria from Tg mAPP mice to be resistant to swelling, means that blocking CypD expression in neurons can be used to treat AD. Certain embodiments of the invention are directed to the use of antisense nucleotides and small interfering RNA to treat AD by reducing expression of CypD.

The addition of cyclosporin A (CsA), a specific inhibitor of CypD, attenuated mitochondrial swelling in Tg mAPP mice (FIG. 3A2-3). The rate of swelling was significantly faster in mitochondria isolated from Tg mAPP mice than in mitochondria isolated from the double knock-out mutant Tg mAPP/CypD−/− mice or their nonTg littermates (FIG. 3B1-3). These results support the conclusion that interaction between amyloid beta and CypD causes swelling, and shows that blocking the formation of the Aβ/CypD complex has therapeutic utility in treating AD. Therefore certain embodiments are directed to administering therapeutic amounts of cyclosporin A or cyclosporin D, or biologically active analogs, derivatives, fragments or variants thereof to treat or prevent AD and PD. Another agent, that can be used to inhibit CypD is the cyclosporine A analog NIM811.

To more carefully evaluate mitochondrial function, we measured inner mitochondrial membrane potential (ΔΨm) and permeability transition in brain slices in situ. Brain slices from Tg mice were loaded with tetramethylrhodamine methyl ester (TMRM) to assess inner mitochondrial ΔΨm and permeability transition. The intensity of TMRM staining was significantly decreased in the temporal cortex and hippocampus from 12-month-old Tg mAPP mice compared to other groups of mice (nonTg, Tg CypD−/− knock-outs, and double Tg mAPP/CypD−/− knock-out). Deletion of CypD in Tg mAPP mice (Tg mAPP/CypD−/− knock-outs) caused the mice to be largely resistant to loss of ΔΨm as was demonstrated by increased TMRM staining intensity compared to single Tg mAPP mice (FIG. 3C1-2). Thus in the absence of CypD, mitochondria were protected from Aβ-mediated swelling and opening of membrane permeability transition pore.

The evidence showing decreased mPTP and generation of reactive oxygen species (ROS) in the single Tg mAPP mice led us to investigate whether Alβ promotes CypD translocation to the inner membrane where the CypD-Aβ interaction occurs. Mitochondrial inner membranes were purified from mouse cerebral cortex, and were then subjected to tris-gel electrophoresis and immunoblotting with α-CypD IgG. There was no significant difference in the amount of CypD translocation to the inner membrane between 3-month-old Tg mAPP and nonTg littermates, whereas, at age of 6 to 12 month-old mice, the intensity of the CypD band was increased significantly in the mitochondrial inner membrane isolated from Tg mAPP mice compared to the mitochondrial inner membrane isolated from nonTg littermates (FIG. 3D-E). No CypD translocation was detected in the mitochondrial inner membrane from the double Tg mAPP/CypD−/− mice (FIG. 3D-E). This age-dependent CypD translocation is consistent with the observation that increased mitochondrial swelling and decreased mitochondrial ΔΨm occurred at the age 6 to 12 months of Tg mAPP mice. Furthermore, CypD-Aβ complex was found in the mitochondrial inner membrane from Tg mAPP mice (FIG. 3G, lane 4), but not in the mitochondrial inner membrane isolated from nonTg (FIG. 3G, lane 2) and the double Tg mAPP/CypD−/− mice (FIG. 3G, lane 3), or Tg mAPP mice in which the preimmune IgG substituted for CypD antibody (FIG. 3G, lane 1). Thus, the CypD-Aβ interaction plays an important role in the function of the mitochondrial permeability transition pore by sequestering CypD. Anti-CypD antibodies that prevent it from forming a complex with amyloid beta can also be used therapeutically to treat or prevent AD, including those that bind to the CypD binding site on amyloid beta. Other diseases associated with elevated CypD levels and/or mitochondrial pathology include ischemia/reperfusion injury, such as stroke and cardiac ischemia; or multiple sclerosis. Antibody therapy to reduce the circulating levels of CypD can be used for all of these diseases.

Because mitochondria are the principal sites of generation of reactive oxygen species (ROS) under physiologic conditions and Aβ is known to trigger oxidative stress, we tested whether CypD-Aβ interaction correlates with generation of ROS in mitochondria. To evaluate mitochondrial ROS generation, brain slices were stained with MitoSox, an indicator for ROS generation in mitochondria. The percentage of area occupied and the intensity of MitoSox staining were both increased significantly in the temporal cortex and hippocampus of Tg mAPP mice as compared with other groups of mice (nonTg, Tg CypD−/−, and double Tg mAPP/CypD−/−) (FIG. 3G-I). It was noted that the levels of ROS were dramatically attenuated in the double mutant Tg mAPP/CypD−/− mice (reduced ˜80% vs. single Tg mAPP mice, FIG. 3G, I), demonstrating that absence of CypD protects from Aβ-mediated mitochondrial ROS generation.

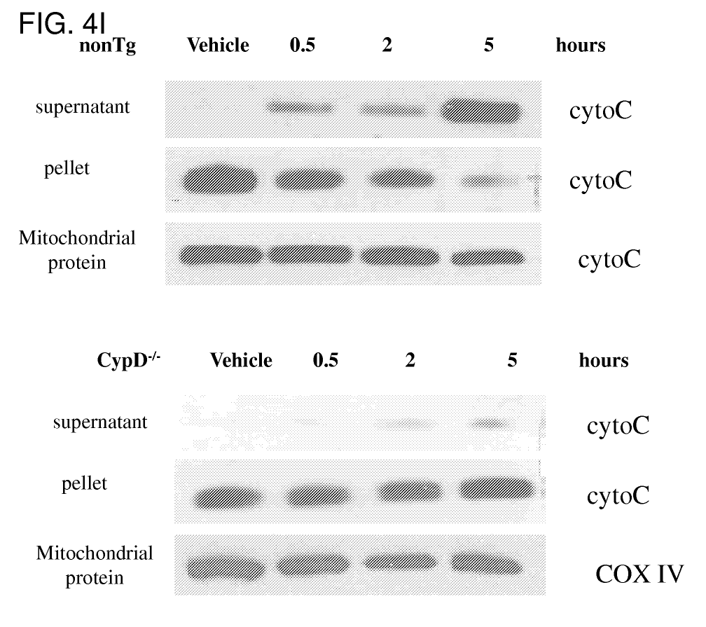

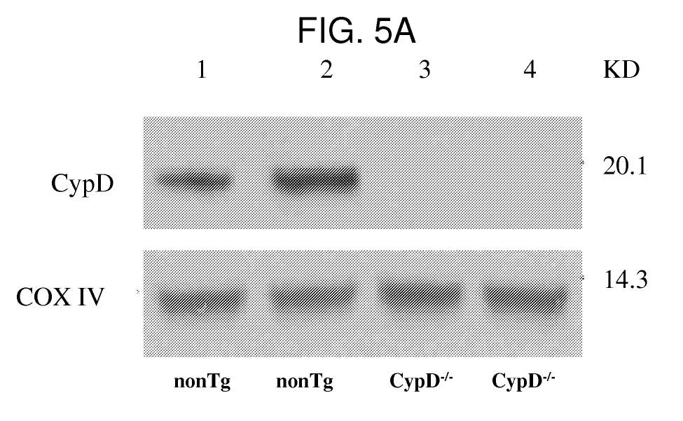

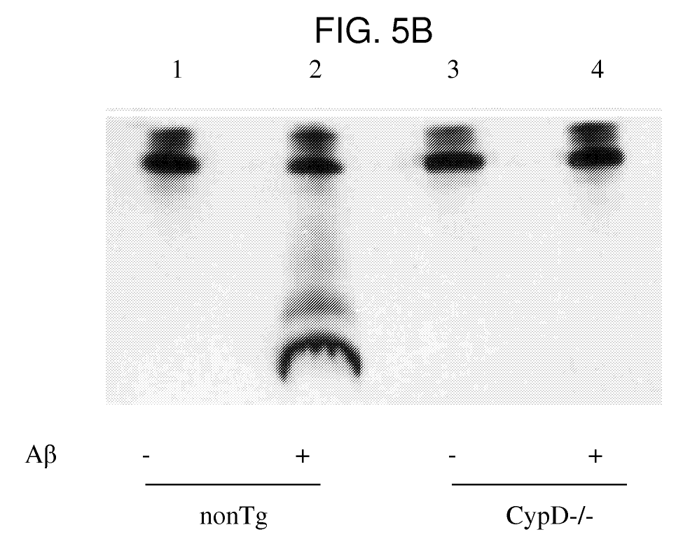

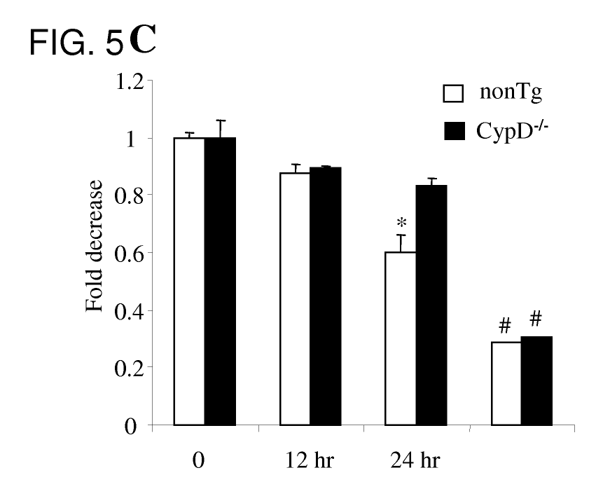

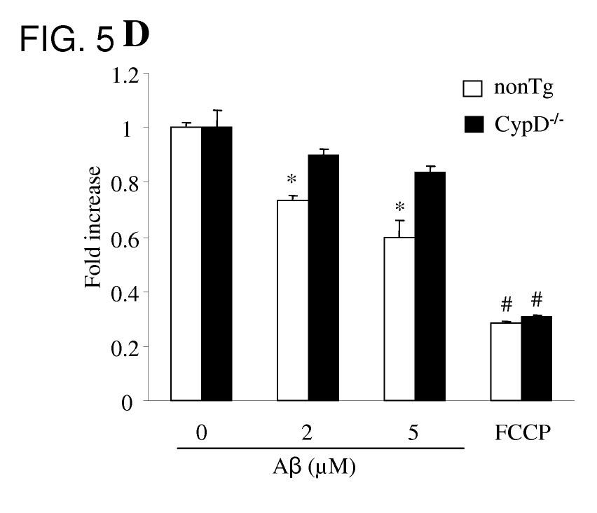

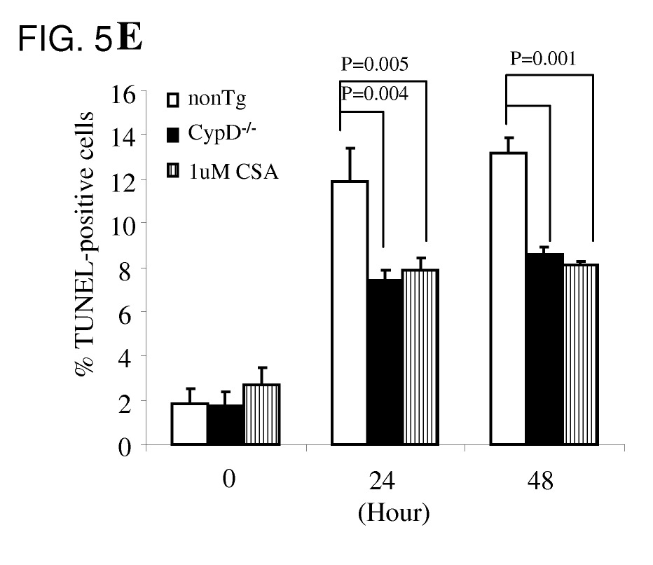

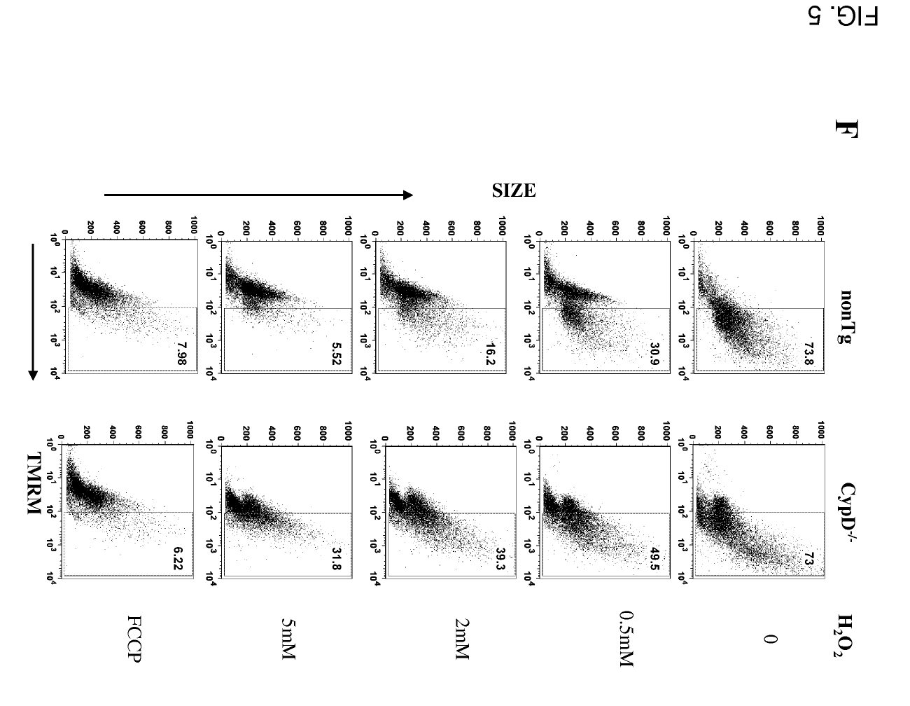

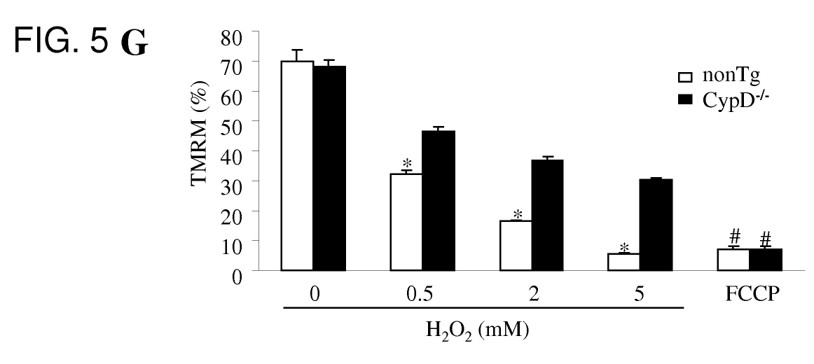

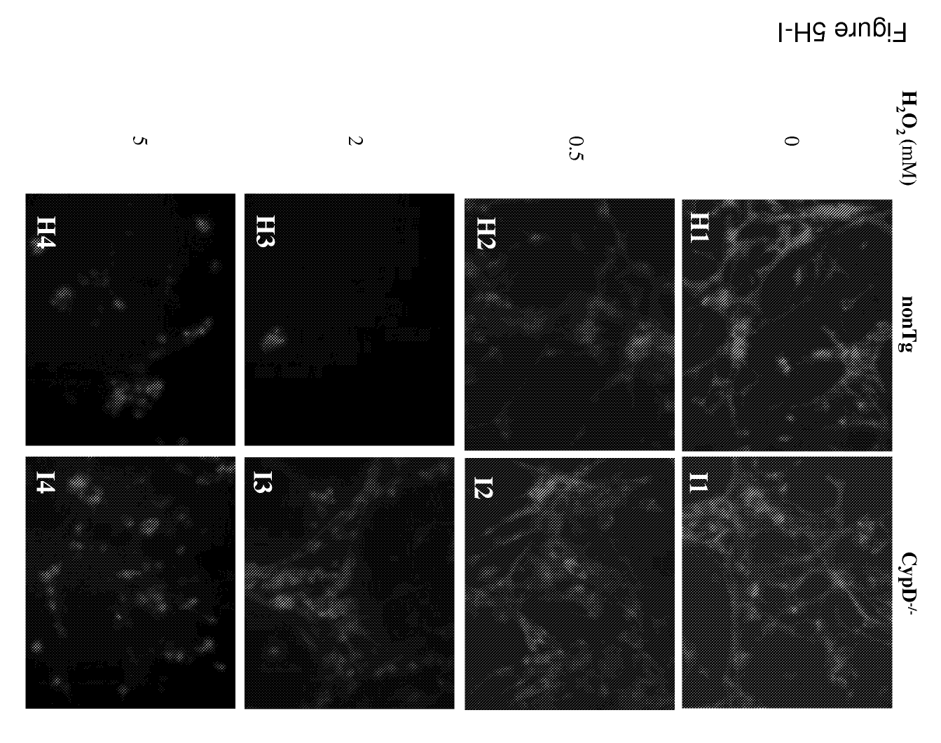

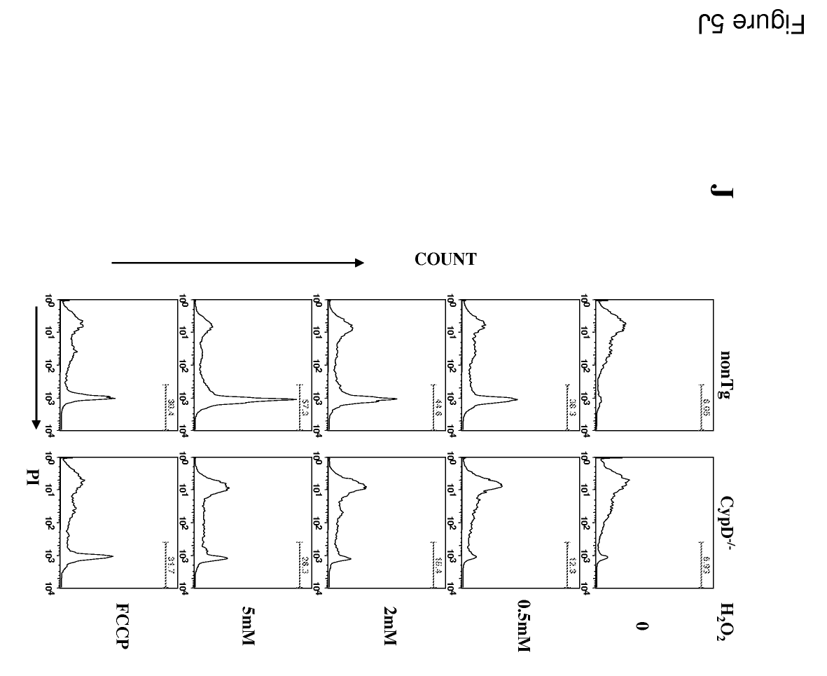

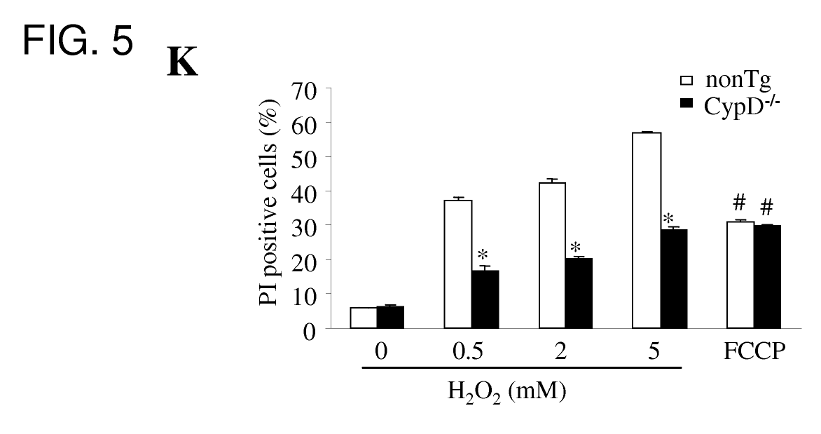

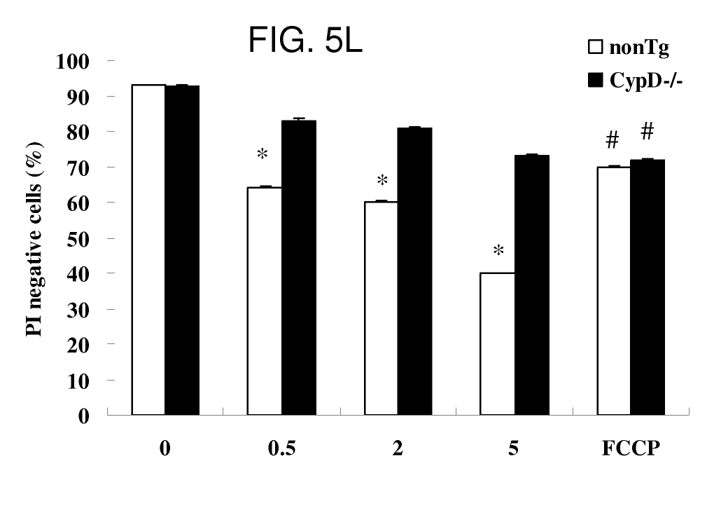

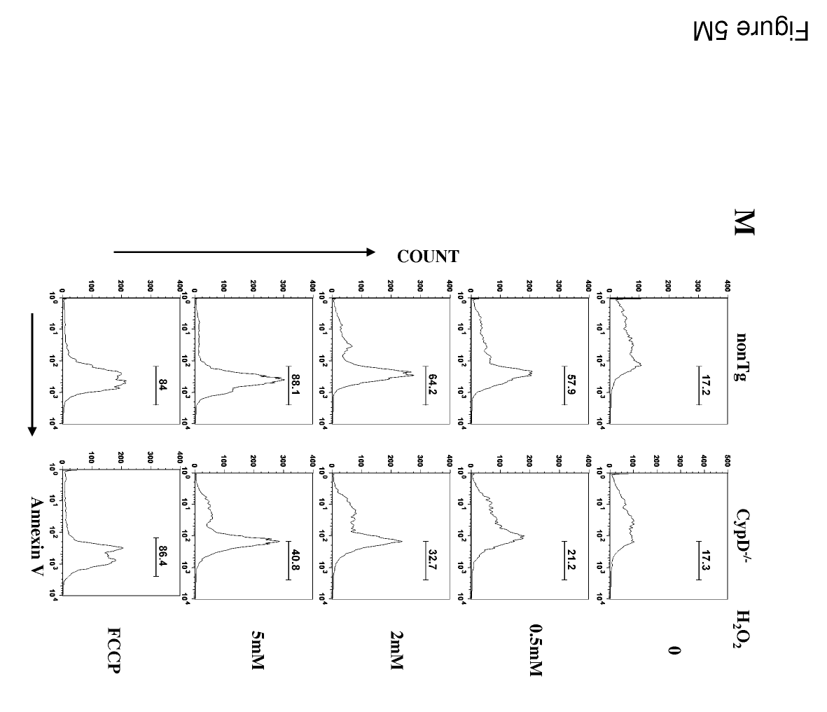

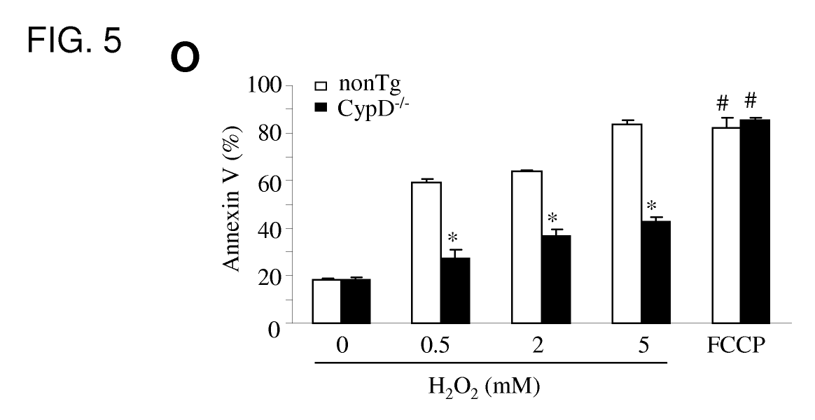

These observations indicate that the absence of CypD protects neurons from Aβ-induced toxicity. Because the interaction of Aβ with CypD enhanced the generation of ROS (which not observed in CypD-deficient mice) (FIG. 3G-I), we carefully evaluated a direct effect of oxidative stress (H2O2) on mitochondrial and neuronal toxicity in the absence of CypD. Fluorescence-activated cell sorting (FACS) showed a dramatic reduction in TMRM fluorescence in CypD-deficient cultured cortical neurons exposed to increasing concentrations of H2O2 (FIG. 5F-G). We observed that CypD−/− neurons were resistant to H2O2-induced loss of ΔΨm as demonstrated by reduction of TMRM-positive neurons from 73% to 32% compared to the nonTg neurons where the reduction went from 73.8% to 5.5%. Confocal microscopy further confirmed that H2O2-treated nonTg neurons showed a significant dose-dependent reduction of TMRM staining, whereas CypD−/− neurons displayed much more TMRM staining than nonTg neurons induced by H2O2 (FIG. 5H, I). Further, FACS analyses showed significant increases in PI- and Annexin V-positive cells (indicators of cell death) following H2O2 treatment (FIG. 5J-M) in nonTg neurons, while CypD−/− neurons were also protected from H2O2-induced cell death (FIG. 5J-M). Certain embodiments are therefore directed to methods to reduce oxidative stress and its consequent damage by normalizing CypD levels, inhibiting CypD activity or blocking it with antibodies.

Next, we assessed mitochondrial function by examining the activity of cytochrome c oxidase, a key enzyme in the respiratory chain, and levels of ATP in the various Tg mice. As previously reported [7, 8], Tg mAPP mice showed a decrease in enzyme activity associated with complex IV of the respiratory chain, and impaired energy metabolism as shown by a reduction in the ATP level compared to nonTg littermate controls at age of 12 months (FIG. 3J-K). The COX IV activity and ATP levels in nonTg mice were comparable to the CypD-deficient mice, showing that deletion of CypD does not interfere with mitochondrial function under physiologic conditions. However, deletion of CypD in the double mutant Tg mAPP/CypD−/− mice dramatically increased mitochondrial enzyme activity (FIG. 3J) up to 80% and abrogated reduction of ATP (FIG. 3K) compared to the single mutant Tg mAPP mice (FIG. 3K). These data indicate that in an Aβ-rich environment, blockade of CypD attenuated or protected against Aβ-mediated mitochondrial dysfunction.

### CypD-Aβ Interaction Mediates Mitochondrial Perturbation In Vitro

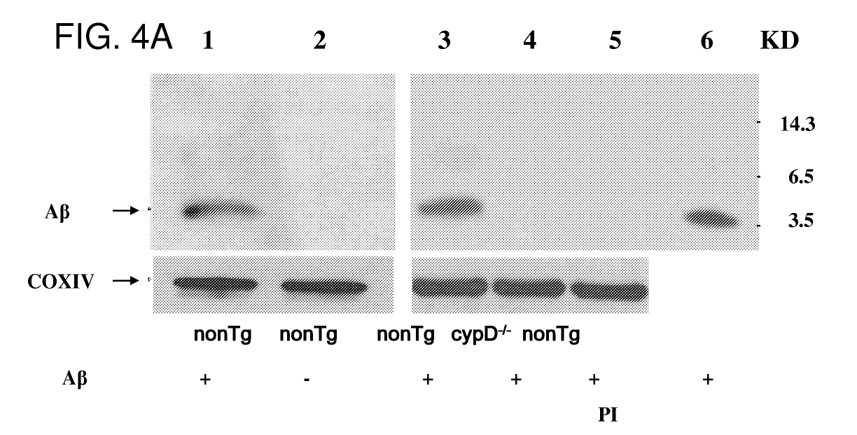

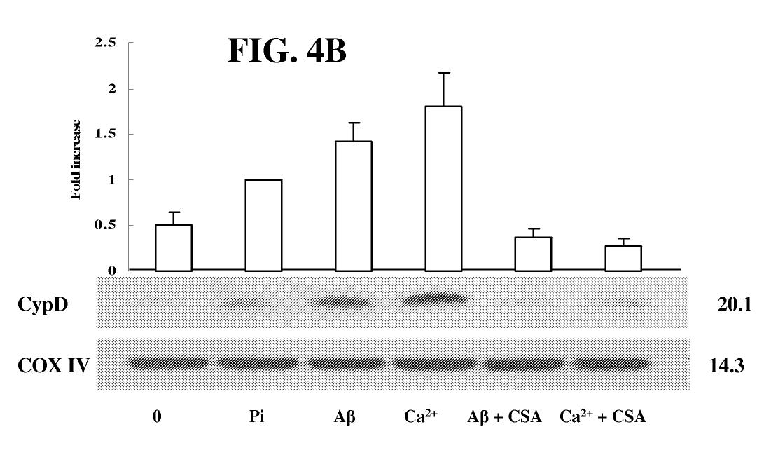

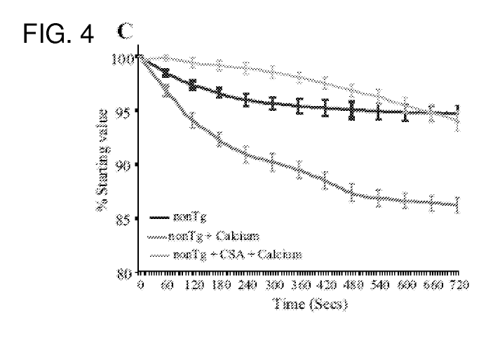

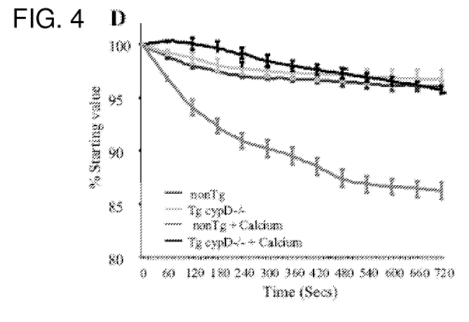

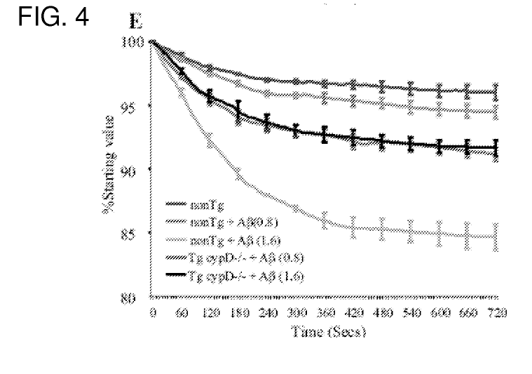

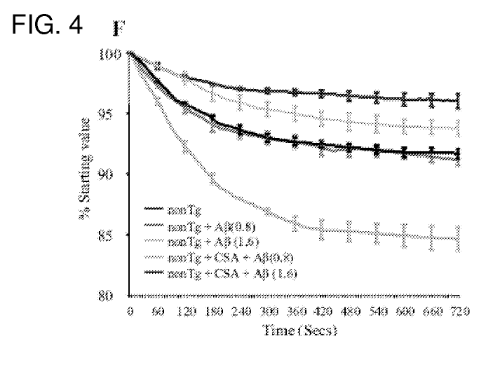

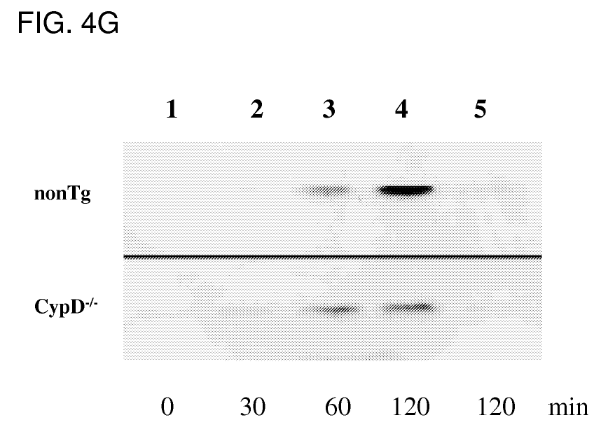

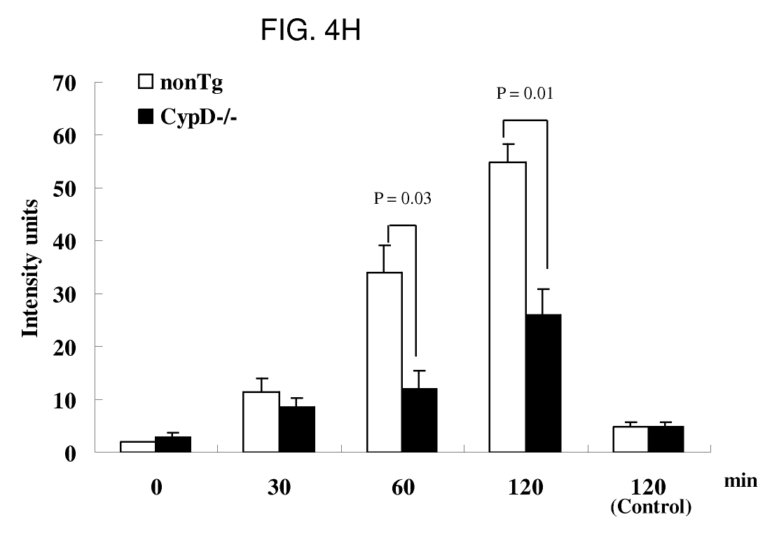

To directly assess the impact of the CypD-Aβ interaction on mitochondrial integrity/properties, we examined the effect of exogenous Aβ on isolated mitochondria from nonTg mice and from Tg CypD-deficient mice. First, CypD-Aβ complex was found in Aβ-treated mitochondria isolated from nonTg mice (FIG. 4A, lanes 1 & 3) but not in the vehicle-treated mitochondria (lane 2), mitochondria lacking CypD (lane 4), or anti-CypD replaced by the preimmune IgG (lane 5) for the immunoprecipitation. This result shows that the specific interaction of CypD with Aβ occurs in normal mitochondria (nonTg mice) exposed to exogenous Aβ. Second, Aβ-treated mitochondria from nonTg mice displayed a significant increase in CypD translocation to the mitochondrial inner membrane compared to the vehicle-treated mitochondria. In the presence of calcium (100 μM), a strong inducer for the binding of CypD to the inner membrane, the CypD immunoreactive band was increased significantly in the mitochondrial inner membrane compared to vehicle-treated mitochondria. The addition of CsA completely blocked CypD translocation to the inner membrane induced by Aβ and Ca2+ (FIG. 4B).

Next, we determined whether Aβ- or Ca2+-mediated CypD translocation is responsible for mPTP formation. Consistent with the previous studies [19, 21], mitochondria isolated from nonTg mice showed Ca2+, -induced swelling, which was efficiently inhibited by addition of the CypD inhibitor CsA. By contrast, mitochondria isolated from Tg CypD-deficient mice were significantly resistant to Ca2+-induced swelling (FIG. 4C-D). Similarly, mitochondria isolated from nonTg mice revealed a does-dependent swelling in response to Aβ compared to the vehicle-treated mitochondria. In contrast, mitochondria lacking CypD showed diminished Aβ-mediated swelling by ˜90% and ˜60% for Aβ (0.8 μM) and for Aβ (1.6 μM), respectively (FIG. 4E). The effect of Aβ on mitochondrial swelling was significantly attenuated by the addition of CsA in all cases (FIG. 4F). CsA (1 μM) only partially rescued Aβ-induced swelling of mitochondria derived from nonTg mice, which was nearly overlaid with the swelling curves seen in CypD-deficient mitochondria. The inhibitory effects of CsA and deletion of CypD on Aβ-mediated swelling show that CypD-dependent mPTP at least in part, involves Aβ-mediated mitochondria toxicity. Consequences of the CypD-Aβ interaction potentiating mPTP were seen as increased levels of cytochrome c release into the supernatant in nonTg-derived mitochondria treated with Aβ (FIG. 4G, lanes 3 & 4; H), compared with control mitochondria that were not treated with Aβ (FIG. 4G, lanes 1 & 5; H). Aβ treatment caused a time-dependent release of cytochrome c into the supernatant from nonTg mitochondria, whereas CypD-deficient mitochondria exposed to Aβ released significantly lower amounts of cytochrome c (FIG. 4G-H). In addition, oxidative stress (H2O2)-mediated cytochrome c release was blocked in CypD-deficient mitochondria in a similar time-dependent manner (FIG. 4I). COX IV was used as a control for the purity of the mitochondrial preparation and equal amount of mitochondrial protein were used in the experiments. Certain other embodiments are directed to the use of CsA or CsD to inhibit CypD thereby reducing oxidative stress.

### CypD-Aβ Interaction Directly Induces Neuronal Death

To determine whether the CypD-Aβ interaction has a direct effect on neuronal damage, we examined the effect of a deficiency of CypD on Aβ-mediated neurotoxicity in primary cultured neurons. Western blot for CypD confirmed expression of CypD in nonTg neurons but not in CypD−/− neurons (FIG. 5A). Immunoprecipitation with α-CypD IgG followed by immunoblotting with α-Aβ detected an Aβ (˜4KD) immunoreactive band in nonTg cortical neurons but not in CypD−/− neurons exposed to Aβ, indicating that CypD is able to form a complex with exogenous Aβ in in vitro cultured neurons (FIG. 5B). Incubation of oligomeric Aβ42 with cultured nonTg cortical neurons reduced membrane potential (ΔΨm) as shown by TMRM staining in a time- and dose-dependent manner. In contrast, CypD−/− neurons showed an attenuated Aβ-induced reduction of ΔΨm (FIG. 5C-D). The addition of carbonyl cyanide p-(trifluoromethoxy) phenylhydrazone (FCCP), a mitochondrial uncoupler, dissipated membrane ΔΨm. Consequently, the percentage of apoptotic cells was increased significantly in Aβ-treated nonTg neurons as compared with the vehicle-treated controls. CypD-deficient neurons displayed an attenuation of apoptosis in the presence of Aβ compared to the nonTg-derived neurons, and the addition of CsA blocked Aβ-apoptosis (FIG. 5E).

CypD Deficiency Improves Spatial Learning, Memory and Neuropathological Changes in Tg mAPP Mice

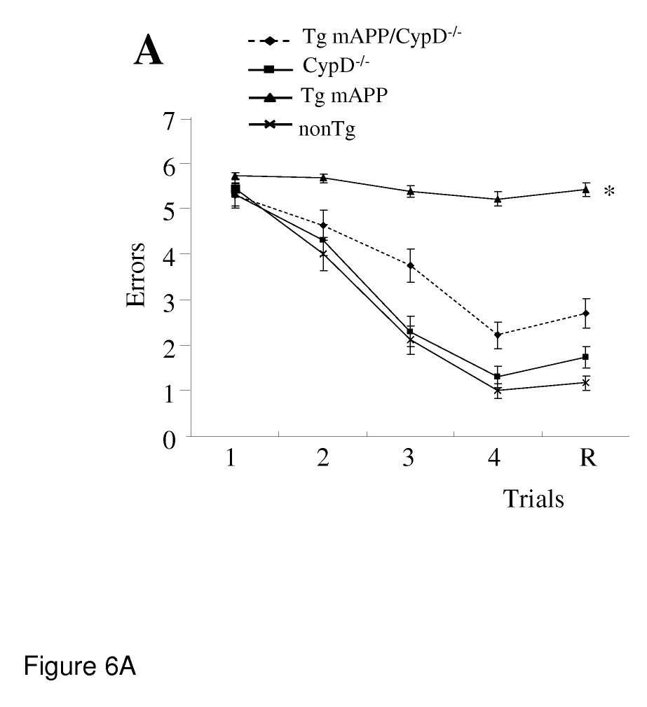

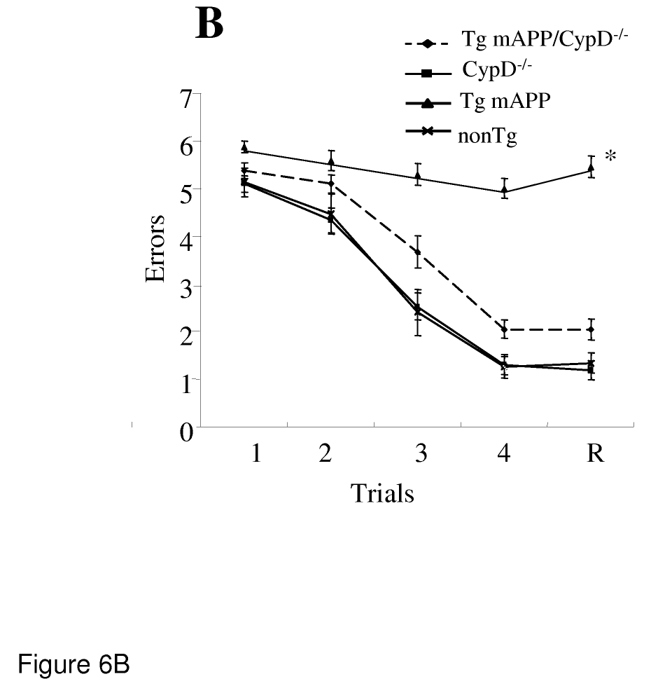

We have demonstrated that CypD-deficiency has a protective effect in Tg mAPP mice, as was measured by the improvement of mitochondrial and neuronal function in double mutant Tg mAPP/CypD−/− mice in vivo and in cultured CypD-deficient neurons treated with Aβ in vitro. Therefore, we sought to determine whether CypD deficiency improves learning and/or memory in the radial arm water maze test that detects hippocampal-dependent learning and memory deficits. At 6 and 12 months of age, CypD-deficient and nonTg mice showed strong learning and memory capacity (FIG. 6A-B). In contrast, Tg mAPP mice displayed impaired spatial memory for platform location between trials (average of about 5-6 errors by trials 4 or 5), as well as during the 30 min delay before trial 5. Spatial learning memory was significantly improved in the double mutant Tg mAPP/CypD−/− mice (˜2-3 errors by trials 4 or 5) compared to Tg mAPP mice (˜5-6 errors by trials 4 or 5) (FIG. 6A-B). The four groups of Tg mice showed no difference in their speed of swimming or in the time required to reach the platform in the visible platform test (not shown). These results indicate the improvement in learning and memory is a consequence of the absence of CypD in Tg mAPP mice.

Behavioral changes were reflected by neuropathologic improvement. Diminished density of cholinergic fibers and synapses is associated with AD-like pathology[22, 23]. Others have shown that acetylcholinesterase (AChE)-positive neurites were reduced significantly in AD-affected regions of the brain in transgenic AD mice [9, 24]. Consistent with the previous reports, we show that Tg mAPP mice at 12 months of age displayed a significant decrease in AChE activity (˜40%) in subiculum compared to the nonTg and CypD-deficient mice. The decreased AChE activity was largely reversed in the double Tg mAPP/CypD −/− mice ˜20% (FIG. 6C), indicating that absence of CypD protects against Aβ-mediated neuropathological changes as shown by increased levels of AChE activity. Therefore certain other embodiments a redirected to reducing memory loss from AD, PD, or aging by reducing CypD expression or activity, or blocking its ability to form a complex with amyloid beta.

### Depletion of CypD Protects Against the Deleterious Effects of Aβ-Soluble Oligomers on Synaptic Function

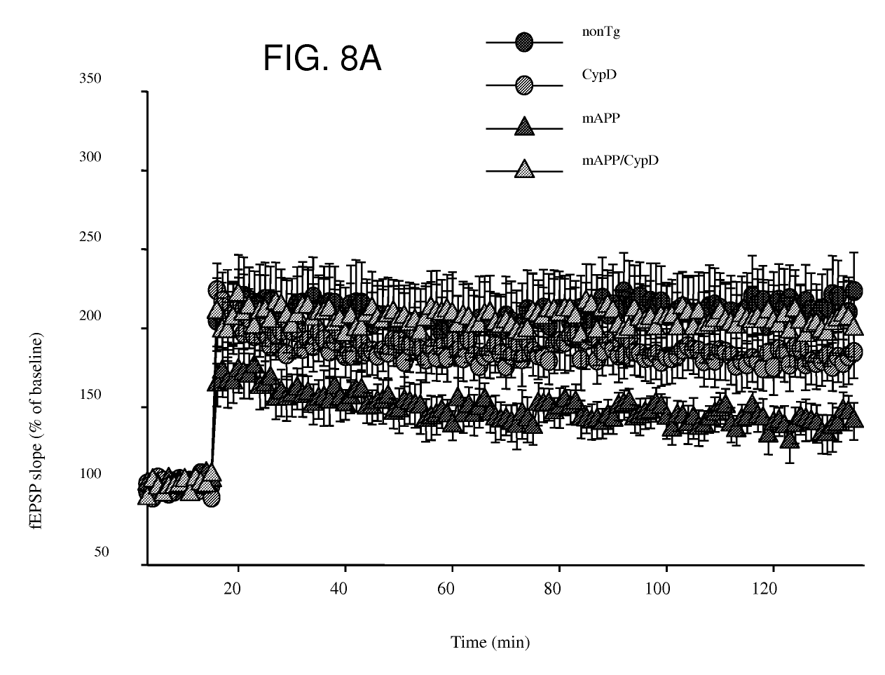

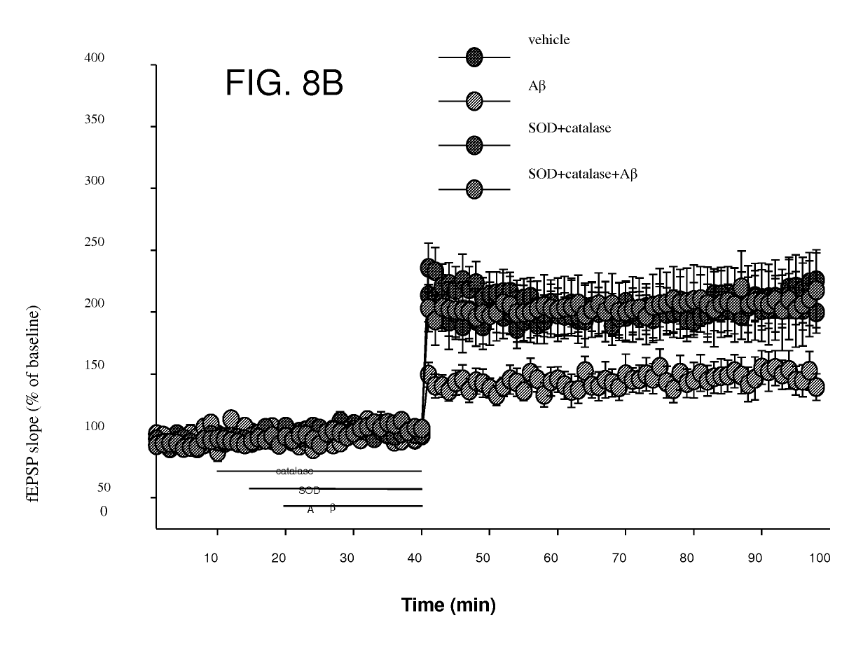

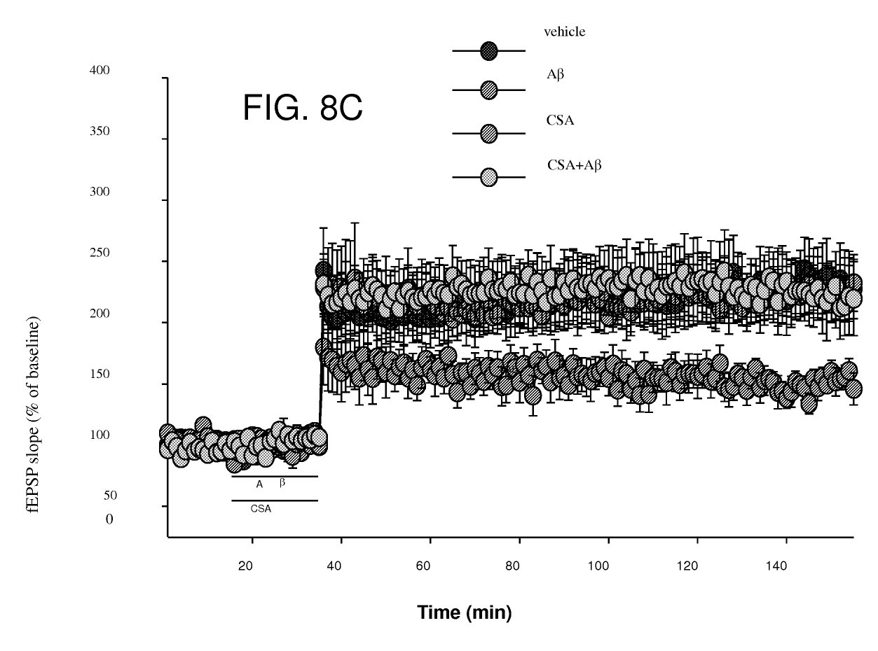

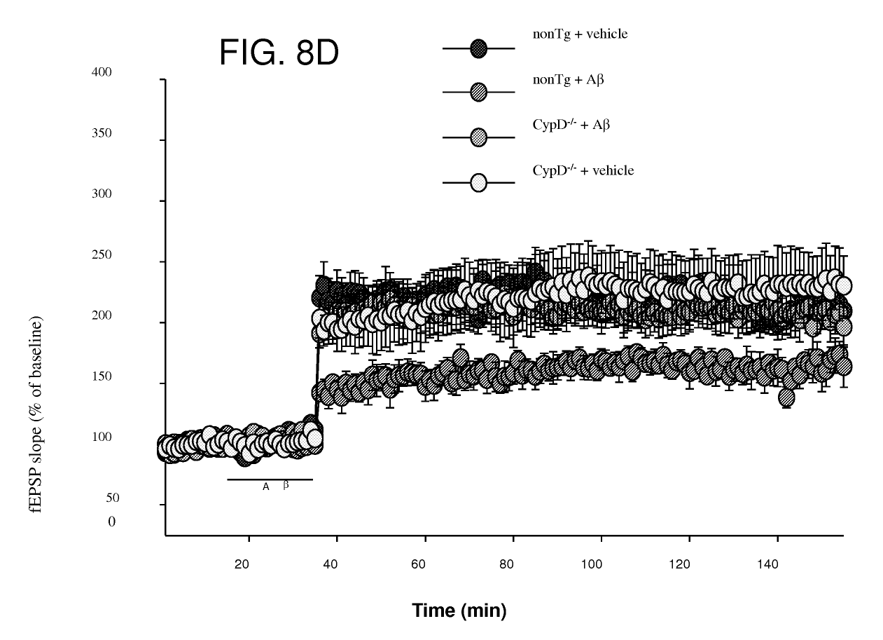

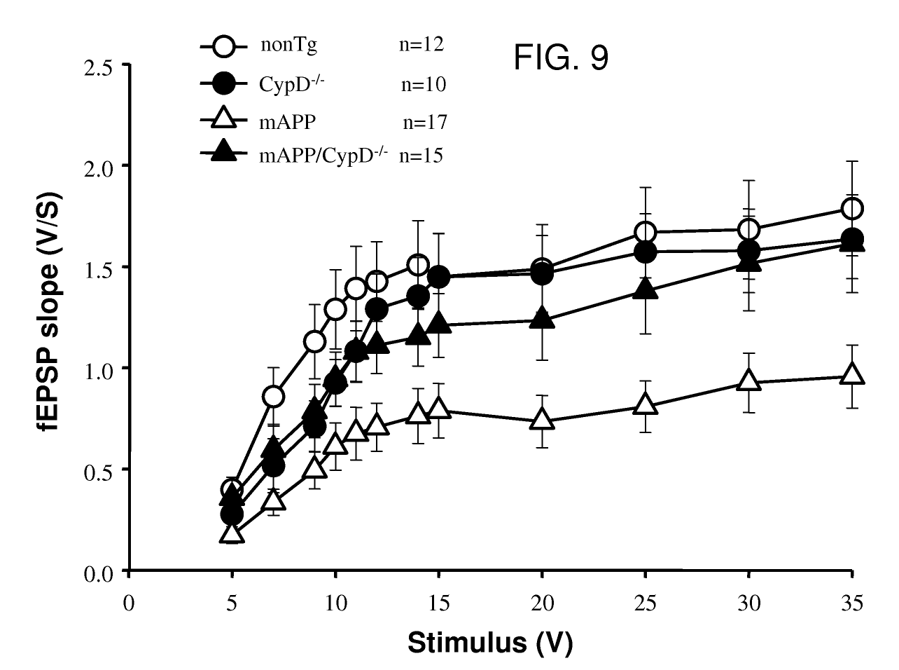

Cognitive abnormalities in AD are thought to be linked to synaptic dysfunction [39]. Given that Tg mAPP/CypD−/− mice showed an improvement of learning and memory, we examined whether these mice had also an improvement of long term potentiation (LTP), a form of synaptic plasticity that is widely studied as a cellular model for learning and memory. Slices from 12-13 month old Tg mAPP mice showed a reduction in LTP compared to slices from non Tg littermates (140.99±11.81 at 120 min after the tetanus vs 218.52±24.38, n=10/12; FIG. 8A. Slices from Tg mAPP/CypD−/− littermates, in turn, displayed normal LTP (199.32±20.01, n=13; FIG. 8B. Tg CypD−/− slices also displayed a normal LTP (184.70±16.47, n=10). Furthermore, Tg mAPP/CypD−/− slices also demonstrated an improvement of BST compared to Tg mAPP slices (FIG. 9). To test a direct effect of CypD deletion on Aβ-mediated reduction of LTP, hippocampal slices from CypD−/− and nonTg mice were treated with Aβ and recorded LTP. We found similar amounts of potentiation in CypD-deficient slices compared to nonTg slices in the presence of vehicle (230.06±24.71, vs 209.39±15.77 in nonTg slices, n=6/7; FIG. 8B However, deficiency of CypD protected hippocampal slices against reduction of LTP by 200 nM oligomeric Aβ42 (206.42±17.35 in Aβ-treated CypD−/− slices vs 163.91±17.36 in Aβ-treated nonTg slices, n=9/7; FIG. 7B [5D]), Similarly, addition of CsA (1 μM), an inhibitor of CypD, rescued reduction of LTP induced by Aβ42 in nonTg hippocampal slices (219.61±30.27 in CsA+Aβ treated slices vs 145.96±13.09 in Aβ alone treated nonTg slices, n=8/7; FIG. 8C). CsA alone did not alter LTP (232.43±23.19 in CsA-treated slices vs 227.57±24.16 in vehicle treated nonTg slices, n=7/6; FIG. 8C. These results confirm previous data showing that Aβ impairs LTP [40]. Most importantly, they indicate that the depletion of CypD may protect against the deleterious effects of Aβ soluble oligomers onto synaptic function.

### Aβ-Mediated Reduction of LTP is Prevented by ROS Scavenging

Since the absence of CypD attenuates generation of ROS in Tg mAPP mice and suppresses reduction of LTP induced by Aβ, we also determined whether Aβ-mediated reduction of LTP can be prevented by ROS scavenging. Addition of 100 U/ml superoxide dismutase (SOD, a superoxide scavenger converting it into oxygen and hydrogen peroxide) plus 260 U/ml catalase (to prevent inhibition of LTP by hydrogen peroxide through its conversion into oxygen and water [41, 42]) blocked Aβ-induced inhibition of LTP in nonTg hippocampal slices (220.89±30.97 in SOD+catalase+Aβ treated slices vs 145.37±12.24 in Aβ alone treated nonTg slices, n=7/8; FIG. 7D [5F]). SOD plus catalase did not alter LTP (205.05±11.79 in SOD+catalase treated slices vs 219.30±24.42 in vehicle treated nonTg slices, n=8/6; FIG. 8D). These experiments show a role of ROS in Aβ-mediated impairment of LTP.

### Effect of CypD Deficiency on Basal Synaptic Transmission (BST)

We further observed that slices from hippocampus region of the brain of 12-13 month old mAPP mice showed a reduction of BST compared to nonTg slices ([F(1,27)=11.01, P=0.0026]. In contrast, depletion of CypD in Tg mAPP mice (Tg mAPP/CypD−/−) protected slices from APP littermates against reduction of BST [F(1,30)=5.159, P=0.0305]. BST was normal in slices from CypD−/− littermates [F(1,20)=0.5476 P=0.4678]. Thus blocking or eliminating CypD prevents the decrease in BST caused by a high APP environment. FIG. 9.

### Pharmaceutical Compositions

Based on and supported by the data presented above, certain embodiments of the present invention provide methods for treating AD or PD, or other disorder associated with abnormally elevated levels of CypD expression. In one embodiment, the method involves administering a therapeutically effective amount of an agent that inhibits or blocks the action of CypD, such as anti-cyclophilin D antibodies or Cyclosporine A or D that blocks the trans-isomerase action of CypD. Alternative therapies include administering therapeutically effective amounts of agents that block the formation of the CypD/Aβ complex, such as anti-cyclophilin D or anti-Aβ antibodies. Yet another embodiment is directed to treating or preventing AD or PD by administering a therapeutically effective amount of an agent that reduces CypD expression, including antisense nucleic acids or si RNA.

The invention encompasses use of the polypeptides, nucleic acids, antibodies and other therapeutic agents described herein formulated in pharmaceutical compositions to administer to a subject, or to target cells or tissues in a subject. Uses are both diagnostic and therapeutic, and for drug screening. The therapeutic agents (also referred to as “active compounds”) can be incorporated into pharmaceutical compositions suitable for administration to a subject, e.g., a human. Such compositions typically comprise the nucleic acid molecule, protein, modulator (Cyclophilin D inhibitor CsA or cyclosporin D), or antibody and a pharmaceutically acceptable carrier. It is understood however, that administration can also be to cells in vitro as well as to in vivo model systems such as non-human transgenic animals, including those described herein. Therapeutically, any method known in the art to decrease Cyclophilin D expression, inhibit CypD activity, or block CypD-Aβ complex formation can be used.

Formulations of cyclosporine or anti-CypD antibodies may contain more than one active compound as necessary for the particular indication being treated, preferably those with complementary activities that do not adversely affect each other. Such molecules are suitably present in combination in amounts that are effective for the purpose intended. Both cyclosporine and anti-CypD antibodies can be in a single formulation.

It is especially advantageous to formulate oral or parenteral compositions in dosage unit form for ease of administration and uniformity of dosage. “Dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

A therapeutically effective amount of antibody, protein or polypeptide or nucleic acid (antisense or si RNA) (i.e., an effective dosage) has been defined. This amount typically varies and can be an amount sufficient to achieve serum therapeutic agent levels typically of between about 1 nanogram per milliliter and about 10 micrograms per milliliter in the subject, or an amount sufficient to achieve serum therapeutic agent levels of between about 1 nanogram per milliliter and about 7 micrograms per milliliter in the subject. Expressed as a daily dose, this amount can be between about 0.1 nanograms per kilogram body weight per day and about 20 milligrams per kilogram body weight per day, or between about 1 nanogram per kilogram body weight per day and about 10 milligrams per kilogram body weight per day. However, the skilled artisan will appreciate that certain factors may influence the dosage required to effectively treat a subject, including but not limited to the severity of the condition, previous treatments, the general health and/or age of the subject, and other disorders or diseases present. Moreover, treatment of a subject with a therapeutically effective amount of a protein, polypeptide, nucleotide or antibody can include a single treatment or, preferably, can include a series of treatments. CsA has been widely used as an immunosuppressant, and these doses can be used as starting points for treatment of the diseases described herein.

Another way to determine a therapeutically effective dose of an agent that reduces CypD expression for the present invention is to determine the amount of active agent (antisense nucleotide or siRNA) needed to reduce the level of CypD in a biological sample from the patient.

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds or therapeutic agents can also be incorporated into the compositions. A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylene diamante tetra acetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. Sterile injectable solutions can be prepared by incorporating the active compound (e.g., cyclosporine A, antisense nucleotides or siRNA, or anti-Cyclophilin or Aβ antibodies) in the required amount in an appropriate solvent with one or a combination of ingredients enumerated above, as required, followed by filtered sterilization.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

If appropriate, the compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems.

It is understood that appropriate doses of the active therapeutic agents depends upon a number of factors within the ken of the ordinarily skilled physician, veterinarian, or researcher. The dose(s) will vary, for example, depending upon the identity, size, and condition of the subject or sample being treated, further depending upon the route by which the composition is to be administered. It is furthermore understood that appropriate doses depend upon the potency of the therapeutic agent with respect to the expression or activity to be modulated. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

### Protein Variants.

Variants of cyclosporine, including preferably cyclosporine A and D for therapeutic use as described herein, include proteins substantially homologous to cyclosporin A or cyclosporin D but derived from another organism, i.e., an ortholog. Variants also include proteins that are substantially homologous to cyclosporin A or cyclosporin D that are produced by chemical synthesis. Variants also include proteins that are substantially homologous to cyclosporines that are produced by recombinant methods.

As used herein, two proteins (or a region of the proteins) are substantially homologous when the amino acid sequences are at least about 70-75%, typically at least about 80-85%, and most typically at least about 90-95%, 97%, 98% or 99% or more homologous. A substantially homologous amino acid sequence, according to the present invention, will be encoded by a nucleic acid sequence hybridizing to the corresponding nucleic acid sequence, or portion thereof, under stringent conditions as more fully described below.

Conservative Amino Acid Substitutions: Aromatic Phenylalanine Tryptophan Tyrosine Hydrophobic Leucine Isoleucine Valine Polar Glutamine Asparagine Basic Arginine Lysine Histidine Acidic Aspartic Acid Glutamic Acid Small Alanine Serine Threonine Methionine Glycine

A variant polypeptide can differ in amino acid sequence by one or more substitutions, deletions, insertions, inversions, fusions, and truncations or a combination of any of these. Variant polypeptides can be fully functional or can lack function in one or more activities.

Fully functional variants typically contain only conservative variation or variation in non-critical residues or in non-critical regions. Functional variants can also contain substitution of similar amino acids, which results in no change or an insignificant change in function. Alternatively, such substitutions may positively or negatively affect function to some degree.

As indicated, variants can be naturally-occurring or can be made by recombinant means of chemical synthesis to provide useful and novel characteristics of the desired protein. This includes preventing immunogenicity from pharmaceutical formulations by preventing protein aggregation.

Substantial homology can be to the entire nucleic acid or amino acid sequence or to fragments of these sequences. Fragments can be derived from the full naturally occurring amino acid sequence. However, the invention also encompasses fragments of the variants of cyclosporin A or cyclosporin D as described herein. Accordingly, a fragment can comprise any length that retains one or more of the biological activities of the protein, for example the ability to inhibit Cyclophilin D binding to AB. Fragments can be discrete (not fused to other amino acids or polypeptides) or can be within a larger polypeptide. Further, several fragments can be comprised within a single larger polypeptide.

Cyclosporin A or cyclosporin D polypeptides can be produced by any conventional means (Houghten, R. A. (1985) Proc. Natl. Acad. Sci. USA 82:5131-5135). Simultaneous multiple peptide synthesis is described in U.S. Pat. No. 4,631,211.

Polypeptides often contain amino acids other than the 20 amino acids commonly referred to as the 20 naturally-occurring amino acids. Further, many amino acids, including the terminal amino acids, may be modified by natural processes, such as processing and other post-translational modifications, or by chemical modification techniques well known in the art. Common modifications that occur naturally in polypeptides are described below.

Accordingly, the polypeptides also encompass derivatives or analogs in which a substituted amino acid residue is not one encoded by the genetic code, in which a substituent group is included, in which the mature polypeptide is fused with another compound, such as a compound to increase the half-life of the polypeptide (for example, polyethylene glycol), or in which the additional amino acids are fused to the mature polypeptide, such as a leader or secretory sequence or a sequence for purification of the mature polypeptide or a pro-protein sequence.

### Protein Modifications

Cyclosporines, and their biologically active analogs, derivatives, fragments and variants for use in the present invention can be modified according to known methods in medicinal chemistry to increase its stability, half-life, uptake or efficacy. Certain known modifications are described below.

As is also well known, polypeptides are not always entirely linear. For instance, polypeptides may be branched as a result of ubiquitination, and they may be circular, with or without branching, generally as a result of post-translation events, including natural processing events and events brought about by human manipulation which do not occur naturally. Circular, branched and branched circular polypeptides may be synthesized by non-translational natural processes and by synthetic methods.

Modifications can occur anywhere in a polypeptide, including the peptide backbone, the amino acid side-chains and the amino or carboxyl termini. Blockage of the amino or carboxyl group in a polypeptide, or both, by a covalent modification, is common in naturally-occurring and synthetic polypeptides. For instance, the amino terminal residue of polypeptides made in E. coli, prior to proteolytic processing, almost invariably will be N-formylmethionine.

The modifications can be a function of how the protein is made. For recombinant polypeptides, for example, the modifications will be determined by the host cell posttranslational modification capacity and the modification signals in the polypeptide amino acid sequence. Accordingly, when glycosylation is desired, a polypeptide should be expressed in a glycosylating host, generally a eukaryotic cell. Insect cells often carry out the same posttranslational glycosylations as mammalian cells, and, for this reason, insect cell expression systems have been developed to efficiently express mammalian proteins having native patterns of glycosylation. Similar considerations apply to other modifications. The same type of modification may be present in the same or varying degree at several sites in a given polypeptide. Also, a given polypeptide may contain more than one type of modification.

Cyclosporines can be isolated and purified from cells that naturally express it, purified from cells that naturally express it but have been modified to overproduce osteocalcin, e.g., purified from cells that have been altered to express it (recombinant), synthesized using known protein synthesis methods, or by modifying cells that naturally encode cyclosporine to express it.

| Protein Modification | Description |
|---|---|
| Acetylation | Acetylation of N-terminus or e-lysines. Introducing an acetyl |
| | group into a protein, specifically, the substitution of an acetyl |
| | group for an active hydrogen atom. |
| | A reaction involving the replacement of the hydrogen atom of a |
| | hydroxyl group with an acetyl group (CH₃CO) yields a specific |
| | ester, the acetate. Acetic anhydride is commonly used as an |
| | acetylating agent, which reacts with free hydroxyl groups. |
| | Acylation may facilitate addition of other functional groups. A |
| | common reaction is acylation of e.g., conserved lysine residues |
| | with a biotin appendage. |
| ADP-ribosylation | Covalently linking proteins or other compounds via an arginine- |
| | specific reaction. |
| Alkylation | Alkylation is the transfer of an alkyl group from one molecule to |
| | another. The alkyl group may be transferred as an alkyl |
| | carbocation, a free radical or a carbanion (or their equivalents). |
| | Alkylation is accomplished by using certain functional groups |
| | such as alkyl electrophiles, alkyl nucleophiles or sometimes |
| | alkyl radicals or carbene acceptors. A common example is |
| | methylation (usually at a lysine or arginine residue). |
| Amidation | Reductive animation of the N-terminus. Methods for amidation |
| | of insulin are described in U.S. Pat. No. 4,489,159. |
| Carbamylation | Nigen et al. describes a method of carbamylating hemoglobin. |
| Carboxylation | Carboxylation typically occurs at the glutamate residues of a |
| | protein, which may be catalyzed by a carboxylase enzyme (in |
| | the presence of Vitamin K - a cofactor). |
| Citrullination | Citrullination involves the addition of citrulline amino acids to |
| | the arginine residues of a protein, which is catalyzed by |
| | peptidylarginine deaminase enzymes (PADs). This generally |
| | converts a positively charged arginine into a neutral citrulline |
| | residue, which may affect the hydrophobicity of the protein (and |
| | can lead to unfolding). |
| Condensation of amines | Such reactions, may be used, e.g., to attach a peptide to other |
| with aspartate or glutamate | proteins labels. |
| Covalent attachment of | Flavin mononucleotide (FAD) may be covalently attached to |
| flavin | serine and/or threonine residues. May be used, e.g., as a light- |
| | activated tag. |
| Covalent attachment of | A heme moiety is generally a prosthetic group that consists of |
| heme moiety | an iron atom contained in the center of a large heterocyclic |
| | organic ring, which is referred to as a porphyrin. The heme |
| | moiety may be used, e.g., as a tag for the peptide. |
| Attachment of a nucleotide | May be used as a tag or as a basis for further derivatising a |
| or nucleotide derivative | peptide. |
| Cross-linking | Cross-linking is a method of covalently joining two proteins. |
| | Cross-linkers contain reactive ends to specific functional |
| | groups (primary amines, sulfhydryls, etc.) on proteins or other |
| | molecules. Several chemical groups may be targets for |
| | reactions in proteins and peptides. For example, Ethylene |
| | glycol bis[succinimidylsuccinate, Bis[2- |
| | (succinimidooxycarbonyloxy)ethyl]sulfone, and |
| | Bis[sulfosuccinimidyl] suberate link amines to amines. |
| Cyclization | For example, cyclization of amino acids to create optimized |
| | delivery forms that are resistant to, e.g., aminopeptidases (e.g., |
| | formation of pyroglutamate, a cyclized form of glutamic acid). |
| Disulfide bond formation | Disulfide bonds in proteins are formed by thiol-disulfide |
| | exchange reactions, particularly between cysteine residues (e.g., |
| | formation of cystine). |
| Demethylation | See, e.g., U.S. Pat. No. 4,250,088 (Process for demethylating lignin). |
| Formylation | The addition of a formyl group to, e.g., the N-terminus of a |
| | protein. See, e.g., U.S. Pat. Nos. 4,059,589, 4,801,742, and |
| | 6,350,902. |
| Glycylation | The covalent linkage of one to more than 40 glycine residues to |
| | the tubulin C-terminal tail. |
| Glycosylation | Glycosylation may be used to add saccharides (or |
| | polysaccharides) to the hydroxy oxygen atoms of serine and |
| | threonine side chains (which is also known as O-linked |
| | Glycosylation). Glycosylation may also be used to add |
| | saccharides (or polysaccharides) to the amide nitrogen of |
| | asparagine side chains (which is also known as N-linked |
| | Glycosylation), e.g., via oligosaccharyl transferase. |
| GPI anchor formation | The addition of glycosylphosphatidylinositol to the C-terminus |
| | of a protein. GPI anchor formation involves the addition of a |
| | hydrophobic phosphatidylinositol group - linked through a |
| | carbohydrate containing linker (e.g., glucosamine and mannose |
| | linked to phosphoryl ethanolamine residue) - to the C-terminal |
| | amino acid of a protein. |
| Hydroxylation | Chemical process that introduces one or more hydroxyl groups |
| | (—OH) into a protein (or radical). Hydroxylation reactions are |
| | typically catalyzed by hydroxylases. Proline is the principal |
| | residue to be hydroxylated in proteins, which occurs at the C^{γ} |
| | atom, forming hydroxyproline (Hyp). In some cases, proline |
| | may be hydroxylated at its C^{β} atom. Lysine may also be |
| | hydroxylated on its C^{δ} atom, forming hydroxylysine (Hyl). |
| | These three reactions are catalyzed by large, multi-subunit |
| | enzymes known as prolyl 4-hydroxylase, prolyl 3-hydroxylase |
| | and lysyl 5-hydroxylase, respectively. These reactions require |
| | iron (as well as molecular oxygen and α-ketoglutarate) to carry |
| | out the oxidation, and use ascorbic acid to return the iron to its |
| | reduced state. |
| Iodination | See, e.g., U.S. Pat. No. 6,303,326 for a disclosure of an enzyme that is |
| | capable of iodinating proteins. U.S. Pat. No. 4,448,764 discloses, e.g., a |
| | reagent that may be used to iodinate proteins. |
| ISGylation | Covalently linking a peptide to the ISG15 (Interferon- |
| | Stimulated Gene 15) protein, for, e.g., modulating immune |
| | response. |
| Methylation | Reductive methylation of protein amino acids with |
| | formaldehyde and sodium cyanoborohydride has been shown to |
| | provide up to 25% yield of N-cyanomethyl (—CH₂CN) product. |
| | The addition of metal ions, such as Ni²⁺, which complex with |
| | free cyanide ions, improves reductive methylation yields by |
| | suppressing by-product formation. The N-cyanomethyl group |
| | itself, produced in good yield when cyanide ion replaces |
| | cyanoborohydride, may have some value as a reversible |
| | modifier of amino groups in proteins. (Gidley et al.) |
| | Methylation may occur at the arginine and lysine residues of a |
| | protein, as well as the N- and C-terminus thereof. |
| Myristoylation | Myristoylation involves the covalent attachment of a myristoyl |
| | group (a derivative of myristic acid), via an amide bond, to the |
| | alpha-amino group of an N-terminal glycine residue. This |
| | addition is catalyzed by the N-myristoyltransferase enzyme. |
| Oxidation | Oxidation of cysteines. |
| | Oxidation of N-terminal Serine or Threonine residues |
| | (followed by hydrazine or aminooxy condensations). |
| | Oxidation of glycosylations (followed by hydrazine or |
| | aminooxy condensations). |
| Palmitoylation | Palmitoylation is the attachment of fatty acids, such as palmitic |
| | acid, to cysteine residues of proteins. Palmitoylation increases |
| | the hydrophobicity of a protein. |
| (Poly)glutamylation | Polyglutamylation occurs at the glutamate residues of a protein. |
| | Specifically, the gamma-carboxy group of a glutamate will form |
| | a peptide-like bond with the amino group of a free glutamate |
| | whose alpha-carboxy group may be extended into a |
| | polyglutamate chain. The glutamylation reaction is catalyzed by |
| | a glutamylase enzyme (or removed by a deglutamylase |
| | enzyme). Polyglutamylation has been carried out at the C- |
| | terminus of proteins to add up to about six glutamate residues. |
| | Using such a reaction, Tubulin and other proteins can be |
| | covalently linked to glutamic acid residues. |
| Phosphopantetheinylation | The addition of a 4′-phosphopantetheinyl group. |
| Phosphorylation | A process for phosphorylation of a protein or peptide by |
| | contacting a protein or peptide with phosphoric acid in the |
| | presence of a non-aqueous apolar organic solvent and contacting |
| | the resultant solution with a dehydrating agent is disclosed e.g., |
| | in U.S. Pat. No. 4,534,894. Insulin products are described to be |
| | amenable to this process. See, e.g., U.S. Pat. No. 4,534,894. Typically, |
| | phosphorylation occurs at the serine, threonine, and tyrosine |
| | residues of a protein. |
| Prenylation | Prenylation (or isoprenylation or lipidation) is the addition of |
| | hydrophobic molecules to a protein. Protein prenylation |
| | involves the transfer of either a farnesyl (linear grouping of |
| | three isoprene units) or a geranyl-geranyl moiety to C-terminal |
| | cysteine(s) of the target protein. |
| Proteolytic Processing | Processing, e.g., cleavage of a protein at a peptide bond. |
| Selenoylation | The exchange of, e.g., a sulfur atom in the peptide for selenium, |
| | using a selenium donor, such as selenophosphate. |
| Sulfation | Processes for sulfating hydroxyl moieties, particularly tertiary |
| | amines, are described in, e.g., U.S. Pat. No. 6,452,035. A process for |
| | sulphation of a protein or peptide by contacting the protein or |
| | peptide with sulphuric acid in the presence of a non-aqueous |
| | apolar organic solvent and contacting the resultant solution with |
| | a dehydrating agent is disclosed. Insulin products are described |
| | to be amenable to this process. See, e.g., U.S. Pat. No. 4,534,894. |
| SUMOylation | Covalently linking a peptide a SUMO (small ubiquitin-related |
| | Modifier) protein, for, e.g., stabilizing the peptide. |
| Transglutamination | Covalently linking other protein(s) or chemical groups (e.g., |
| | PEG) via a bridge at glutamine residues |
| tRNA-mediated addition of | For example, the site-specific modification (insertion) of an |
| amino acids (e.g., | amino acid analog into a peptide. |
| arginylation) | |

### Antibodies and Antibody-Based Assays

“Antibody” or “antibodies” include intact molecules as well as fragments thereof that are capable of specifically binding to an epitope of a protein of interest, including Cyclophilin D and Aβ. An antibody that specifically binds to Cyclophilin D or Aβ that decreases Cyclophilin D/Aβ complex formation or that inactivates CypD can be used therapeutically and diagnostically for AD and PD (and the other diseases described herein), and in drug screening assays. As used herein, “specific binding” refers to the property of the antibody, to: (1) to bind to CypD (or amyloid beta), e.g., human CypD protein, with an affinity of at least 1×107 M-1, and (2) preferentially bind to CypD, e.g., human CypD protein, with an affinity that is at least two-fold, 50-fold, 100-fold, 1000-fold, or more greater than its affinity for binding to a non-specific antigen (e.g., BSA, casein) other than CypD. In a preferred embodiment, the interaction, e.g., binding, between an anti-CypD antibody and CypD occurs with high affinity (e.g., affinity constant of at least 107 M 1, preferably, between 108 M-1 and 1010, or about 109 M-1) and specificity. As used herein, an amount of an anti-CypD antibody effective to prevent a disorder, or a “therapeutically or prophylactically effective amount” of the antibody refers to an amount which is effective, upon single- or multiple-dose administration to the subject, in preventing or delaying the occurrence of the onset or recurrence of AD or PD as described herein, or treating a symptom thereof.

The term “epitope” refers to an antigenic determinant on an antigen to which an antibody binds. Epitopes usually consist of chemically active surface groupings of molecules such as amino acids or sugar side chains, and typically have specific three-dimensional structural characteristics, as well as specific charge characteristics. Epitopes generally have at least five contiguous amino acids.

The terms “antibody” and “antibodies” include polyclonal antibodies, monoclonal antibodies, humanized or chimeric antibodies, single chain Fv antibody fragments, Fab fragments, and F(ab).sub.2 fragments. Polyclonal antibodies are heterogeneous populations of antibody molecules that are specific for a particular antigen, while monoclonal antibodies are homogeneous populations of antibodies to a particular epitope contained within an antigen. Monoclonal antibodies are particularly useful.

Antibody fragments that have specific binding affinity for the polypeptide of interest can be generated by known techniques. Such antibody fragments include, but are not limited to, F(ab′).sub.2 fragments that can be produced by pepsin digestion of an antibody molecule, and Fab fragments that can be generated by deducing the disulfide bridges of F(ab′) 2 fragments. Alternatively, Fab expression libraries can be constructed. See, for example, Huse et al. (1989) Science 246:1275-1281. Single chain Fv antibody fragments are formed by linking the heavy and light chain fragments of the Fv region via an amino acid bridge (e.g., 15 to 18 amino acids), resulting in a single chain polypeptide. Single chain Fv antibody fragments can be produced through standard techniques, such as those disclosed in U.S. Pat. No. 4,946,778.

Once produced, antibodies or fragments thereof can be tested for recognition of the target polypeptide by standard immunoassay methods including, for example, enzyme-linked immunosorbent assay (ELISA) or radioimmunoassay assay (RIA). See, Short Protocols in Molecular Biology eds. Ausubel et al., Green Publishing Associates and John Wiley & Sons (1992). Suitable antibodies typically have equal binding affinities for recombinant and native proteins.

The term “monospecific antibody” refers to an antibody that displays a single binding specificity and affinity for a particular target, e.g., epitope. This term includes a “monoclonal antibody” or “monoclonal antibody composition,” which as used herein refer to a preparation of antibodies or fragments thereof of single molecular composition.

The term “recombinant” antibody, as used herein, refers to antibodies that are prepared, expressed, created or isolated by recombinant means, such as antibodies expressed using a recombinant expression vector transfected into a host cell, antibodies isolated from a recombinant, combinatorial antibody library, antibodies isolated from an animal (e.g., a mouse) that is transgenic for human immunoglobulin genes or antibodies prepared, expressed, created or isolated by any other means that involves splicing of human immunoglobulin gene sequences to other DNA sequences. Such recombinant antibodies include humanized, CDR grafted, chimeric, deimmunized, in vitro generated (e.g., by phage display) antibodies, and may optionally include constant regions derived from human germline immunoglobulin sequences.

Human monoclonal antibodies (mAbs) directed against human proteins can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856 859; Green, L. L. et al. 1994 Nature Genet. 7:13 21; Morrison, S. L. et al. 1994 Proc. Natl. Acad. Sci. USA 81:6851 6855; Bruggeman et al. 1993 Year Immunol 7:33 40; Tuaillon et al. 1993 PNAS 90:3720 3724; Bruggeman et al. 1991 Eur J Immunol 21:1323 1326).

Anti-CypD antibodies or fragments thereof useful in the present invention may also be recombinant antibodies produced by host cells transformed with DNA encoding immunoglobulin light and heavy chains of a desired antibody. Recombinant antibodies may be produced by known genetic engineering techniques. For example, recombinant antibodies may be produced by cloning a nucleotide sequence, e.g., a cDNA or genomic DNA sequence, encoding the immunoglobulin light and heavy chains of the desired antibody from a hybridoma cell that produces an antibody useful in this invention. The nucleotide sequence encoding those polypeptides is then inserted into expression vectors so that both genes are operatively linked to their own transcriptional and translational expression control sequences. The expression vector and expression control sequences are chosen to be compatible with the expression host cell used. Typically, both genes are inserted into the same expression vector. Prokaryotic or eukaryotic host cells may be used.

Expression in eukaryotic host cells is preferred because such cells are more likely than prokaryotic cells to assemble and secrete a properly folded and immunologically active antibody. However, any antibody produced that is inactive due to improper folding may be renaturable according to well known methods (Kim and Baldwin, “Specific Intermediates in the Folding Reactions of Small Proteins and the Mechanism of Protein Folding”, Ann. Rev. Biochem. 51, pp. 459 89 (1982)). It is possible that the host cells will produce portions of intact antibodies, such as light chain dimers or heavy chain dimers, which also are antibody homologs according to the present invention.

It will be understood that variations on the above procedure are useful in the present invention. For example, it may be desired to transform a host cell with DNA encoding either the light chain or the heavy chain (but not both) of an antibody. Recombinant DNA technology may also be used to remove some or all of the DNA encoding either or both of the light and heavy chains that is not necessary for CypD binding, e.g., the constant region may be modified by, for example, deleting specific amino acids. The molecules expressed from such truncated DNA molecules are useful in the methods of this invention. In addition, bifunctional antibodies may be produced in which one heavy and one light chain are anti-CypD antibody and the other heavy and light chain are specific for an antigen other than CypD, or another epitope of CypD.

Chimeric antibodies, including chimeric immunoglobulin chains, can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fe constant region is substituted (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041 1043); Liu et al. (1987) PNAS 84:3439 3443; Liu et al., 1987, J. Immunol. 139:3521 3526; Sun et al. (1987) PNAS 84:214 218; Nishimura et al., 1987, Canc. Res. 47:999 1005; Wood et al. (1985) Nature 314:446 449; and Shaw et al., 1988, J. Natl Cancer Inst. 80:1553 1559).

An antibody or an immunoglobulin chain can be humanized by methods known in the art. Once the murine antibodies are obtained, the variable regions can be sequenced. The location of the CDRs and framework residues can be determined (see, Kabat, E. A., et al. (1991) Sequences of Proteins of Immunological Interest, Fifth Edition, U.S. Department of Health and Human Services, NIH Publication No. 91 3242, and Chothia, C. et al. (1987) J. Mol. Biol. 196:901 917, which are incorporated herein by reference). The light and heavy chain variable regions can, optionally, be ligated to corresponding constant regions

The immunoassays, immunohistochemistry, RIA, IRMAs used herein are based on the generation of various antibodies, including those that specifically bind to Cyclophilin D and amyloid beta, or their variants or fragments.

Methods for using antibodies as disclosed herein are particularly applicable to the cells, tissues and disorders that differentially express Cyclophilin D and Aβ or that are involved in conditions as otherwise discussed herein. The methods use antibodies that selectively bind to the protein of interest and its variants and fragments. For therapeutic applications, antibodies that recognize Cyclophilin D and Aβ and Cyclophilin D/Aβ complex formation are preferred. An antibody is considered to selectively or specifically bind, even if it also binds to other proteins that are not substantially homologous with the protein of interest. These other proteins share homology with a fragment or domain of the protein of interest. This conservation in specific regions gives rise to antibodies that bind to both proteins by virtue of the homologous sequence. In this case, it would be understood that antibody binding to the protein of interest is still selective.

The amount of an antigen in a biological sample may be determined by a radioimmunoassay, an immunoradiometric assay, and/or an enzyme immunoassay. “Radioimmunoassay” is a technique for detecting and measuring the concentration of an antigen using a labeled (i.e. radioactively labeled) form of the antigen. Examples of radioactive labels for antigens include H3, C14, and I125. The concentration of antigen (i.e. Cyclophilin D) in a sample (i.e. biological sample) is measured by having the antigen in the sample compete with a labeled (i.e. radioactively) antigen for binding to an antibody to the antigen. To ensure competitive binding between the labeled antigen and the unlabeled antigen, the labeled antigen is present in a concentration sufficient to saturate the binding sites of the antibody. The higher the concentration of antigen in the sample, the lower the concentration of labeled antigen that will bind to the antibody.

In a radioimmunoassay, to determine the concentration of labeled antigen bound to antibody, the antigen-antibody complex must be separated from the free antigen. One method for separating the antigen-antibody complex from the free antigen is by precipitating the antigen-antibody complex with an anti-isotype antiserum. Another method for separating the antigen-antibody complex from the free antigen is by precipitating the antigen-antibody complex with formalin-killed S. aureus. Yet another method for separating the antigen-antibody complex from the free antigen is by performing a “solid-phase radioimmunoassay” where the antibody is linked (i.e. covalently) to Sepharose beads, polystyrene wells, polyvinylchloride wells, or microtiter wells. By comparing the concentration of labeled antigen bound to antibody to a standard curve based on samples having a known concentration of antigen, the concentration of antigen in the biological sample can be determined.

An “Immunoradiometric assay” (IRMA) is an immunoassay in which the antibody reagent is radioactively labeled. An IRMA requires the production of a multivalent antigen conjugate, by techniques such as conjugation to a protein e.g., rabbit serum albumin (RSA). The multivalent antigen conjugate must have at least 2 antigen residues per molecule and the antigen residues must be of sufficient distance apart to allow binding by at least two antibodies to the antigen. For example, in an IRMA the multivalent antigen conjugate can be attached to a solid surface such as a plastic sphere. Unlabeled “sample” antigen and antibody to antigen which is radioactively labeled are added to a test tube containing the multivalent antigen conjugate coated sphere. The antigen in the sample competes with the multivalent antigen conjugate for antigen antibody binding sites. After an appropriate incubation period, the unbound reactants are removed by washing and the amount of radioactivity on the solid phase is determined. The amount of bound radioactive antibody is inversely proportional to the concentration of antigen in the sample.

The most common enzyme immunoassay is the “Enzyme-Linked Immunosorbent Assay (ELISA).” The “Enzyme-Linked Immunosorbent Assay (ELISA)” is a technique for detecting and measuring the concentration of an antigen using a labeled (i.e. enzyme linked) form of the antibody.

In a “sandwich ELISA”, an antibody (i.e. to Cyclophilin D) is linked to a solid phase (i.e. a microtiter plate) and exposed to a biological sample containing antigen (i.e. Cyclophilin D). The solid phase is then washed to remove unbound antigen. A labeled (i.e. enzyme linked) is then bound to the bound-antigen (if present) forming an antibody-antigen-antibody sandwich. Examples of enzymes that can be linked to the antibody are alkaline phosphatase, horseradish peroxidase, luciferase, urease, and .beta.-galactosidase. The enzyme linked antibody reacts with a substrate to generate a colored reaction product that can be assayed for.

In a “competitive ELISA”, antibody is incubated with a sample containing antigen (i.e. Cyclophilin D). The antigen-antibody mixture is then contacted with an antigen-coated solid phase (i.e. a microtiter plate). The more antigen present in the sample, the less free antibody that will be available to bind to the solid phase. A labeled (i.e. enzyme linked) secondary antibody is then added to the solid phase to determine the amount of primary antibody bound to the solid phase.

In an “immunohistochemistry assay” a section of tissue for is tested for specific proteins by exposing the tissue to antibodies that are specific for the protein that is being assayed. The antibodies are then visualized by any of a number of methods to determine the presence and amount of the protein present. Examples of methods used to visualize antibodies are, for example, through enzymes linked to the antibodies (e.g., luciferase, alkaline phosphatase, horseradish peroxidase, or .beta.-galactosidase), or chemical methods (e.g., DAB/Substrate chromagen).

Certain other embodiments are directed to a kit for diagnosing a patient at risk of or having AD, PD or other disease associated with abnormal levels of CypD, for assessing the level of CypD in a biological sample from the patient. The kit includes an antibody that specifically binds to CypD, or biologically active fragment or variant thereof, and reagents for detection of the antibody. In an embodiment the kit contains reagents for detection of the antibody by an enzyme-linked immunosorbent assay. Any antibody that specifically binds to CypD can be used, including antibody fragments as described herein.

### Antisense Nucleic Acids

Other embodiments of the present invention are directed to the use of antisense nucleic acids (either DNA or RNA) or small interfering RNA to reduce or inhibit expression of proteins Cyclophilin D. The antisense nucleic acid can be antisense RNA, antisense DNA or small interfering RNA. The cDNA (encoding the respective genes) sequence encoding human CypD is set forth below. The gene sequence for human CypD is known and is available at Gene bank accession #BC005020, M80254, AAA58434, AAH05020. Based on these known sequences, antisense DNA or RNA that hybridize sufficiently to the respective gene or mRNA encoding CypD to turn off expression can be readily designed and engineered using methods known in the art.

Antisense-RNA and anti-sense DNA have been used therapeutically in mammals to treat various diseases. See for example Agrawal, S. and Zhao, Q. (1998) Curr. Opi. Chemical Biol. Vol. 2, 519-528; Agrawal, S and Zhang, R. (1997) CIBA Found. Symp. Vol. 209, 60-78; and Zhao, Q, et al., (1998), Antisense Nucleic Acid Drug Dev. Vol 8, 451-458; the entire contents of which are hereby incorporated by reference as if fully set forth herein. Antisense oligodeoxyribonucleotides (antisense-DNA) and oligoribonucleotides (antisense-RNA) can base pair with a gene, or its transcript. An antisense PS-oligodeoxyribonucleotide for treatment of cytomegalovirus retinitis in AIDS patients is the first antisense RNA approved for human use in the US. Anderson, K. O., et al., (1996) Antimicrobial Agents Chemother. Vol. 40, 2004-2011, and U.S. Pat. No. 6, 828, 151 by Borchers, et al.

Others have shown that antisense nucleic acids complementary to the gene for glutamine synthetase mRNA in Mtb effectively enter the bacteria, complex with the mRNA and inhibit glutamine synthetase expression, the amount of the poly-L-glutamate/glutamine component in the cell wall, and bacterial replication in vitro. Harth, G., et al., PNAS Jan. 4, 2000, Vol. 97, No. 1, P 418-423, the entire contents of which are hereby incorporated by reference as if fully set forth herein.

Methods of making antisense-nucleic acids are well known in the art. Further provided are methods of modulating the expression of CypD and associated gene and mRNA in cells or tissues by contacting the cells or tissues with one or more of the antisense compounds or compositions of the invention. As used herein, the terms “target nucleic acid” encompass DNA encoding CypD, RNA (including pre-mRNA and mRNA) transcribed from such DNA, and also cDNA derived from such RNA. The specific hybridization of a nucleic acid oligomeric compound with its target nucleic acid interferes with the normal function of the target nucleic acid. This modulation of function of a target nucleic acid by compounds which specifically hybridize to it is generally referred to as “antisense”. The functions of DNA to be interfered with include replication and transcription. The functions of RNA to be interfered with include all vital functions such as, for example, translocation of the RNA to the site of protein translation, translation of protein from the RNA, and catalytic activity which may be engaged in or facilitated by the RNA. The overall effect of such interference with target nucleic acid function is modulation of the expression of the respective protein. In the context of the present invention, “modulation” means reducing or inhibiting in the expression of the gene or mRNA for CypD. cDNA is the preferred antisense nucleotide.

The targeting process includes determination of a site or sites within the target gene or mRNA encoding the CypD for the antisense interaction to occur such that the desired inhibitory effect is achieved. Within the context of the present invention, a preferred intragenic site is the region encompassing the translation initiation or termination codon of the open reading frame (ORF) of the gene. Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene. However routine experimentation will determine the optimal sequence of the antisense or siRNA.

It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which is known in the art to refer to the region between the translation initiation codon and the translation termination codon, is also a region which may be targeted effectively. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene.

It is also known in the art that variants can be produced through the use of alternative signals to start or stop transcription and that pre-mRNAs and mRNAs can possess more that one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites.

Once one or more target sites have been identified, nucleic acids are chosen which are sufficiently complementary to the target, i.e., hybridize sufficiently well and with sufficient specificity, to give the desired effect of inhibiting gene expression and transcription or mRNA translation.

In the context of this invention, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. “Complementary,” as used herein, refers to the capacity for precise pairing between two nucleotides. For example, if a nucleotide at a certain position of a nucleic acid is capable of hydrogen bonding with a nucleotide at the same position of a DNA or RNA molecule, then the nucleic acid and the DNA or RNA are considered to be complementary to each other at that position. The nucleic acid and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the nucleic acid and the DNA or RNA target. Various conditions of stringency can be used for hybridization as is described below. It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1 6.3.6, which is incorporated by reference. Aqueous and nonaqueous methods are described in that reference and either can be used. Specific hybridization conditions referred to herein are as follows: 1) low stringency hybridization conditions in 6.times.sodium chloride/sodium citrate (SSC) at about 45.degree. C., followed by two washes in 0.2.times.SSC, 0.1% SDS at least at 50.degree. C. (the temperature of the washes can be increased to 55.degree. C. for low stringency conditions); 2) medium stringency hybridization conditions in 6.times.SSC at about 45.degree. C., followed by one or more washes in 0.2.times.SSC, 0.1% SDS at 60.degree. C.; 3) high stringency hybridization conditions in 6.times.SSC at about 45.degree. C., followed by one or more washes in 0.2.times.SSC, 0.1% SDS at 65.degree. C.; and preferably 4) very high stringency hybridization conditions are 0.5M sodium phosphate, 7% SDS at 65.degree. C., followed by one or more washes at 0.2.times.SSC, 1% SDS at 65.degree. C. Very high stringency conditions (4) are the preferred conditions and the ones that should be used unless otherwise specified.

Antisense nucleic acids have been employed as therapeutic moieties in the treatment of disease states in animals and man. Antisense nucleic acid drugs, including ribozymes, have been safely and effectively administered to humans and numerous clinical trials are presently underway. It is thus established that nucleic acids can be useful therapeutic modalities that can be configured to be useful in treatment regimes for treatment of cells, tissues and animals, especially humans, for example to regulate expression of Cyclophilin D and AB.

Nucleic acids in the context of this invention includes “oligonucleotides”, which refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

While antisense nucleic acids are a preferred form of antisense compound, the present invention comprehends other oligomeric antisense compounds, including but not limited to oligonucleotide mimetics. The antisense compounds in accordance with this invention preferably comprise from about 8 to about 50 nucleobases (i.e. from about 8 to about 50 linked nucleosides). Particularly preferred antisense compounds are antisense nucleic acids comprising from about 12 to about 30 nucleobases. Antisense compounds include ribozymes, external guide sequence (EGS) nucleic acids (oligozymes), and other short catalytic RNAs or catalytic nucleic acids which hybridize to the target nucleic acid and modulate its expression.

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare nucleic acids such as the phosphorothioates and alkylated derivatives.

The antisense compounds of the invention are synthesized in vitro and do not include antisense compositions of biological origin, or genetic vector constructs designed to direct the in vivo synthesis of antisense molecules. The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos.: 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The antisense compounds of the present invention can be utilized for diagnostics, therapeutics, and prophylaxis and as research reagents and kits.

### Small Interfering RNA

U.S. Patent Application 20040023390 (the entire contents of which are hereby incorporated by reference as if fully set forth herein) teaches that double-stranded RNA (dsRNA) can induce sequence-specific posttranscriptional gene silencing in many organisms by a process known as RNA interference (RNAi). However, in mammalian cells, dsRNA that is 30 base pairs or longer can induce sequence-nonspecific responses that trigger a shut-down of protein synthesis and even cell death through apoptosis. Recent work shows that RNA fragments are the sequence-specific mediators of RNAi (Elbashir et al., 2001). Interference of gene expression by these small interfering RNA (siRNA) is now recognized as a naturally occurring strategy for silencing genes in C. elegans, Drosophila, plants, and in mouse embryonic stem cells, oocytes and early embryos (Cogoni et al., 1994; Baulcombe, 1996; Kennerdell, 1998; Timmons, 1998; Waterhouse et al., 1998; Wianny and Zernicka-Goetz, 2000; Yang et al., 2001; Svoboda et al., 2000).

In mammalian cell culture, a siRNA-mediated reduction in gene expression has been accomplished by transfecting cells with synthetic RNA nucleic acids (Caplan et al., 2001; Elbashir et al., 2001). The 20040023390 application, the entire contents of which are hereby incorporated by reference as if fully set forth herein, provides exemplary methods using a viral vector containing an expression cassette containing a pol II promoter operably-linked to a nucleic acid sequence encoding a small interfering RNA molecule (siRNA) targeted against a gene of interest.

As used herein RNAi is the process of RNA interference. A typical mRNA produces approximately 5,000 copies of a protein. RNAi is a process that interferes with or significantly reduces the number of protein copies made by an mRNA of the targeted protein, CypD. For example, a double-stranded short interfering RNA (siRNA) molecule is engineered to complement and match the protein-encoding nucleotide sequence of the target mRNA to be interfered with. Following intracellular delivery, the siRNA molecule associates with an RNA-induced silencing complex (RISC). The siRNA-associated RISC binds the target mRNA through a base-pairing interaction and degrades it. The RISC remains capable of degrading additional copies of the targeted mRNA. Other forms of RNA can be used such as short hairpin RNA and longer RNA molecules. Longer molecules cause cell death, for example by instigating apoptosis and inducing an interferon response. Cell death was the major hurdle to achieving RNAi in mammals because dsRNAs longer than 30 nucleotides activated defense mechanisms that resulted in non-specific degradation of RNA transcripts and a general shutdown of the host cell. Using from about 20 to about 29 nucleotide siRNAs to mediate gene-specific suppression in mammalian cells has apparently overcome this obstacle. These siRNAs are long enough to cause gene suppression but not of a length that induces an interferon response.

### Drug Screening

Certain embodiments of the invention are directed to cell-based and non-cell based methods of drug screening to identify candidate agents that reduce Cyclophilin D expression, and reduce the ability of Cyclophilin D to bind to and form a complex with Aft The invention provides methods and compositions for screening for bioactive agents which regulate the level of expression of the gene for Cyclophilin D. To develop a specific inhibitor of the CypD-Aβ interaction in in vivo animal models and in vitro cultured neurons, we will add the cell membrane transduction domain of the human immunodeficiency virus-1 (HIV-1) Tat-protein (Tyr-Gly-Arg-Lys-Lys-Arg-Arg-Gln-Arg-Arg-Arg) to the N-terminus of CypD (R V I P S F M C Q A G D F T N H N G T G G K S), called “TAT-CypD-DP” (decoy peptide). Thus the CypD binding region will be cloned and overexpressed in these cells.

The subject assays can be both non-cell based and cell-based. Non-cell based assays for identifying agents that affect gene expression are very well known. They generally involve (a) contacting a transformed or recombinant cell that has a mutant of a native allele encoding a reporter of gene expression of one (or more) of the various proteins, wherein the expression of the reporter is under the control of the native gene expression regulatory sequences of the native allele, with a candidate agent under conditions whereby but for the presence of the agent, the reporter is expressed at a first expression level; and, (b) measuring the expression of the reporter to obtain a second expression level, wherein a difference between the first and second expression levels indicates that the candidate agent modulates expression of one of the gene.

Transgenic animals are useful in screening therapeutic compounds suspected to reduce CypD expression or activity or its ability to complex with Aβ as a means of identifying new drugs. Clearly, cell lines derived from these animals can also be used for the same purpose by assaying for the CAT or LACZ or Luciferase or GFP or other reporter enzyme.

Libraries of Bioactive Agents (of synthetic or natural compounds) for use in drug screening are known in the art. The term “bioactive agent” or “exogenous compound” as used herein includes any molecule, e.g., protein, oligopeptide, small organic molecule, polysaccharide, polynucleotide, lipid, etc., or mixtures thereof, with the capability of directly or indirectly altering the bioactivity of one of the various proteins (CypD or amyloid beta). Bioactive agent agents encompass numerous chemical classes, though typically they are organic molecules, preferably small organic compounds having a molecular weight of more than 100 and less than about 2,500 daltons. Bioactive agent agents comprise functional groups necessary for structural interaction with proteins, particularly hydrogen bonding, and typically include at least an amine, carbonyl, hydroxyl or carboxyl group, preferably at least two of the functional chemical groups. The bioactive agent agents often comprise cyclical carbon or heterocyclic structures and/or aromatic or polyaromatic structures substituted with one or more of the above functional groups. Bioactive agent agents are also found among biomolecules including peptides, saccharides, fatty acids, steroids, purines, pyrimidines, derivatives, structural analogs or combinations thereof. Particularly preferred are peptides.

The invention also provides vectors (also called constructs) containing nucleic acids such as those described above. As used herein, a “vector” is a replicon, such as a plasmid, phage, or cosmid, into which another DNA segment may be inserted so as to bring about the replication of the inserted segment. The vectors of the invention can be expression vectors, preferably including CypD or the CypD fragment that includes the gene encoding the amyloid beta binding sequence. An “expression vector” is a vector that includes one or more expression control sequences, and an “expression control sequence” is a DNA sequence that controls and regulates the transcription and/or translation of another DNA sequence. An expression vector can include a tag sequence designed to facilitate subsequent manipulation of the expressed nucleic acid sequence (e.g., purification or localization). Tag sequences, such as green fluorescent protein (GFP), glutathione S-transferase (GST), polyhistidine, c-myc, hemagglutinin, or Flag tag (Kodak, New Haven, Conn.) sequences typically are expressed as a fusion with the encoded polypeptide. Such tags can be inserted anywhere within the polypeptide including at either the carboxyl or amino terminus.

Another embodiment is directed to an expression construct containing the CypD polypeptide fragment (R V I P S F M C Q A G D F T N H N G T G G K S) that defines the binding region to amyloid beta. In other embodiments biological agents are mixed with the region of CypD polypeptide (R V I P S F M C Q A G D F T N H N G T G G K S) we identified as that which binds to Aβ forming the CypD/Aβ complex to identify agents that block formation of the CypD/AB complex that are useful therapeutically to treat AD and PD.

### Sequence Listings

Full nucleic acid and amino acid sequence listings relevant to this application are listed below. Antisense and small interfering RNAs for use in reducing expression of Cyclophilin D and amyloid beta, can be made that specifically hybridize to the gene and mRNA encoding Cyclophilin D.

### EXAMPLES

### Example 1

### Expression of CypD is Elevated in the Human AD Brain and in Brains of Transgenic APP Mice

To determine whether the expression of CypD is elevated in the human AD brain and in brains of transgenic APP mice, we made polyclonal anti-human CypD IgG which was purified on a protein A column. Antibody against CypD was prepared by immunizing a rabbit with full length human CypD protein. These antibodies were specific for human and mouse CypD and were used for immunoprecipitation, immunoblotting, immunohistochemistry, and functional studies. To make recombinant human CypD protein, we expressed a GST-fusion CypD protein in E. coli, cleaved with thrombin, and purified it to homogeneity. On SDS-PAGE, purified recombinant CypD migrated as a single band under reduced conditions which was confirmed by immunoblotting. Protein sequencing produced a single N-terminal sequence confirming to the sequence deduced from the CypD cDNA. This purified recombinant Human CypD protein was used as antigen to immune rabbit to produce a specific antibody to cypD. This antibody is designated SDY-1. These antibodies were specific for human and mouse CypD and were used for immunoprecipitation, immunoblotting, immunohistochemistry, and functional studies. One embodiment of the present invention is directed to this polyclonal anti-CypD antibody and its therapeutic use to treat AD.

Since CypD is located inside the cell, anti-CypD was bound to TAT peptide to facilitate its entry into the neuron where it can bind to and CypD and prevent it from forming a complex with amyloid beta. This is a recently developed technology in which an 11-amino acid transduction domain of HIV-transactivator protein (TAT) was fused with the protein (antibody) of interest. We have fused TAT to ABAD-DP peptide or its reverse version ABAD-RP peptide to allow their rapid transduction into intact tissue. The protein transduction domain embedded in the HIV TAT protein (31-41) has been successfully used to study intracellular mechanisms based on delivery of peptides/polypeptides delivered with high efficiency (100%) both in vitro and in vivo. It has been shown that TAT-linked protein can go through the blood brain barrier. In addition, an important aspect of TAT-mediated delivery of proteins/peptides is the potential for future therapeutic relevance. In preliminary experiments we demonstrated that these peptides go inside the cells both in the live animal and in the in vitro preparation. In addition, to concentrate CypD-DP in mitochondria in which CypD-Aβ forms a complex, mitochondria targeting sequence derived from the precursor of subunit VIII of human cytochrome C was added to TAT-C D-DP.

### Example 2

### All Species of Aβ Bind to CypD

To study binding of CypD to Aβ, we expressed a GST-fusion protein in E. coli, cleaved with thrombin, and purified it to homogeneity. On SDS-PAGE, purified recombinant CypD migrated as a single band under reduced conditions (FIG. 2A, lane 1), which was confirmed by immunoblotting with specific antibody to CypD (FIG. 2A, lane 2). Protein sequencing produced a single N-terminal sequence conforming to the sequence deduced from the CypD cDNA. Binding studies were employed by the surface plasmon resonance (SPR) in which Aβ was immobilized and CypD was in the mobile phase. Aβ(1-40) and Aβ(1-42), including monomeric and oligomeric Aβ, were used in the binding assay. The kinetic parameters evaluating binding affinity were analyzed as described in the Examples. As shown in FIG. 2B-E, binding was dose-dependent. The evaluating curves (red line) were overlaid with experimental data. The equilibrium dissociation constant KDs for monomeric and oligomeric Aβ40, and monomeric and oligomeric Aβ42 are 1.7 μM, 227 nM, 164 nM, and 4 nM, respectively. These data indicate that all species of Aβ bind to CypD, and that oligomeric Aβ has a higher binding affinity than monomeric Aβ.

To determine if CypD and Aβ actually interacted in the AD brain, we first performed immunoprecipitation-immunoblotting studies using human AD brain. Mitochondria were isolated from the temporal cortex of AD and ND control brains. The purity of mitochondrial preparations was confirmed by the enrichment of cytochrome c oxidase IV (COX IV) (FIG. 2F1, lower panel). Mitochondrial proteins were subjected to immunoprecipitation using α-CypD IgG followed by immunoblotting with α Aβ IgG. Immunoreactive bands of Mr ˜4 kDa were detected in mitochondrial fractions from AD brains, which is consistent with the presence of the CypD-Aβ complex (FIG. 2F1, lanes 5-7). In an age-matched and nondemented (ND) brain, there was virtually no detectable or very little CypD-Aβ complex. (FIG. 2F1, lanes 1-3). Substitution of nonimmune IgG for □-CypD IgG used for immunoprecipitation prevented appearance of the band (FIG. 2F1, lane 8). Densitometric analysis of all immunoreactive bands combined using an NIH image program revealed a greatly increased CypD-Aβ complex in cortical mitochondria from the AD brain compared to the cortical mitochondria from ND brain controls (FIG. 2F2).

### Example 3

### Generation and Characterization of Transgenic Mice Expressing Mutant APP and Deficiency of CypD.

Animal studies were approved by the Animal Care and Use Committee of Columbia University in accordance with the National Institutes of Health guidelines for animal care. CypD homozygous null mice (CypD−/−), as previously described, were obtained from Dr. Jeffery D. Molkentin[19]. These animals have been backcrossed 6 times into the C57BL6 background. Deficiency of CypD was verified by immunoblotting using specific anti-CypD IgG (generated in our laboratory). Transgenic (Tg) mice overexpressing a mutant human form of amyloid precursor protein (mAPP) that encodes hAPP695, hAPP751, and hAPP770 bearing mutations linked to familial AD (V717F, K670M, N671L, J-20 line), driven by the platelet-derived growth factor B-chain promoter, in C57BL6 background have been described previously [9, 25]. Tg mAPP and Tg CypD−/− were crossed to generate four genotypes of mice: double transgenics overexpressing mutant APP and deficiency of CypD (Tg mAPP/CypD−/−), single transgenics overexpressing mutant APP (Tg mAPP), CypD-deficient mice (Tg CypD−/−) and nonTg littermate controls. Offspring of Tg mice were identified by PCR using primers for each specific transgene.

### Example 4

CypD Forms a Complex with Aβ in the Brain Mitochondria From Both AD and Transgenic APP Mice

Mitochondria were isolated from the cerebral cortex of 12-month-old mice expressing mAPP (mAPP mice), CypD knockout mice (CypD−/−), double Tg mice expressing mAPP and deficiency of CypD (mAPP/CypD−/−), and nontransgenic littermate controls (nonTg) (FIG. SA-B). Mitochondrial preparations were evaluated based on enrichment of COX IV as shown in the lower panel of FIG. 2G1. The presence of CypD-Aβ complex was observed in mitochondrial protein extracts from Tg mAPP mice using a similar immunoprecipitation-immunoblotting protocol to that described above (FIG. 2G1, lanes 4-5, upper). Immunoprecipitation with α-CypD IgG followed by immunoblot with α-Aβ IgG revealed a strong Aβ (˜4 Kd) immunoreactive band in mitochondrial fractions from Tg mAPP mice (FIG. 2G1, lane 4-5, upper). In contrast, no immunoreactive bands were observed in mitochondrial extracts from an age- and strain-matched double Tg mAPP/CypD−/− mice, CypD−/−, and nonTg littermates (FIG. 2G1, lanes 1-3, upper). When the preimmune IgG was substituted for the α-CypD in the immunoprecipitation analysis, the immunoreactive band disappeared (data not shown). Quantification of intensity of all immunoreactive bands revealed the presence CypD-Aβ complex only in brain mitochondria from Tg mAPP mice (FIG. 2G2). These results indicate that CypD forms a complex with Aβ in the brain mitochondria from both AD and transgenic APP mice.

Colocalization of CypD and Aβ and their interaction in mitochondria were further confirmed by confocal and electron microscopy. In the cerebral cortex of AD patients, images of α-Aβ (FIG. 2H, red) and α-CypD(FIG. 2H; green), detecting endogenous Aβ and CypD, extensively colocalized (FIG. 2H, yellow). Similarly, in the cerebral cortex of Tg mAPP mice, there was an extensive overlap of immunoreactive CypD and Aβ (FIG. 2I). Immunogold electron microscopy with gold-conjugated antibody was performed on the AD, ND brains (FIG. 2J-K) (postmortem time ˜4 hrs), and on a 12-month-old Tg mAPP mouse brain (FIG. 2L). Sections were stained with colloidal gold-conjugated antibodies specific for the Aβ42 (18 nm gold particles) and for CypD (12 nm gold particles). The results demonstrated particles of both sizes associated with mitochondria (FIG. 2J-L). Preadsorption of antibodies with the respective antigens, Aβ42 or CypD, prevented appearance of gold particles associated with mitochondria (not shown). There was no staining observed when specific antibodies (Aβ42 or CypD) were replaced by the nonimmune IgG (data not shown). These results provided further evidence of the presence of CypD and Aβ colocalization within mitochondria. Colocalization of CypD with Aβ, at least in part, within mitochondria of both in AD brain and transgenic mice, is consistent with the likelihood that CypD-Aβ interaction occurs within mitochondria in vivo.

The expression of CypD in age-related human brain and nonTg mice. Mitochondria were isolated from human brain (A) of temporal pole grey matter of young (33.75+2.19, n=4), aged non-demented ND controls (81.4+3.52, n=9), and AD brain (85.8+1.23, n=12) and nonTg brains in 3 and 12 months of age (2M). Mitochondrial extracts (30 μg per lane) were subjected to the SDA-PAGE followed by immunoblotting with rabbit anti-CypD IgG. Densitometry was performed to quantify the intensity of CypD immunoreactive bands using NIH image program. Immunoblotting of the same preparations of mitochondrial fractions with anti-COX IV was used as protein loading control showing an equal amount of mitochondrial protein loaded to each lane. Data were presented as the fold increase as compared to young controls (2N) or to 3 months of age nonTg mice (B). Both nonTg and Tg mAPP mice at 12 months of age displayed an increase in CypD expression. The levels of brain CypD in 12 months old nonTg mice were significantly higher than that in 3 months old nonTg mice. # P<0.01 vs. 3-month-old nonTg mice. Further, 12-month-old Tg mAPP mice showed a greater increase in CypD levels as compared with 3-month-old nonTG or Tg mAPP. * P<0.01 vs. 3-month-old Tg mAPP mice. There was no significant difference of CypD expression between nonTg and Tg mAPP mice at age of 3 months (P=0.27).

### Example 5

Human Brain Tissues From Patients with AD and Age-Matched, Non-Demented Controls.

Human brain tissues of temporal cortex and hippocampus from patients with AD and aged matched/non-demented controls (ND) were obtained from New York Brain Bank at Columbia University. The averages of age are 85.4+1.2 and 82.4+1.6 for AD and ND, respectively. The averages of postmortem time are 5.4+1.2 and 7+1.2 for AD and ND, respectively.

### Example 6

### Isolation of Mitochondria

Mitochondria were isolated from AD brain or the brains (cortex) from Tg mice as our previously described [8, 9]. The highly purified mitochondria were used for the immunoblotting and immunoprecipitation assay. For the mitochondrial function assay, mitochondria were isolated as described below. Briefly, brain homogenates were centrifuged at 1,500 g for 5 min at 4° C. Supernatant was adjusted to 10% Percoll and centrifuged at 12,000 g for 10 min. The mitochondrial pellet was resuspended in the isolation buffer containing 0.01% digitonin and recentrifuged at 6,000 g for 10 min. Protein concentration was determined by the Bio-Rad DC protein assay (BioRad Laboratories, Hercules, Calif.).

### Example 7

### Quantitative Real-Time PCR and Immunoblotting for CypD.

Total RNA was extracted from the cerebral cortex using TRIzol reagent (Invitrogen, CA). Total RNA (150 ng) was used for the synthesis of cDNA with TagMan Reverse Transcription Reagents kit (Roche Applied Biosystems). A total of RNA (18s gene transcripts) was set as internal controls. Real-time PCR was performed in an ABI Prism 7900 Sequence Detection System (Applied Biosystems) with TaqMan PCR Master Mix. The PCR primers [5′-GCACAGGAGGGAGGTCCAT-3′ and 5′-GCC CCA CAT GCT TCA GTGT-3′ (reverse)] and the probe (6FAM-AAGCCGCTTTCCCGAC-MGBNFQ) were used for mouse CypD NM_{—}134084). The PCR reactions were subjected to 50° C. for 2 min, 95° C. for 10 min, and followed by 40 cycles with 95° C. for 15 seconds and 60° C. for one minute. The relative amount of mRNA level was calculated using the formula 2-ΔΔCt as instructed by the manufacturer. Data are expressed as fold-increase over the nonTg controls (“1.0”) in each FIG.

### Example 8

Immunoprecipitation/Immunoblotting for Detection of CypD-Aβ Complex in Brain Mitochondria from Tg Mice and Human AD Brain.

Mitochondria isolated from cerebral cortex of the Tg mice (N=4-6/group) were resuspended in the buffer (500 μg/ml, 50 mM Tris, 150 mM NaCl, 1 mM EDTA, protease inhibitors, pH 7.5), subjected to the repeatedly freezing-thawing for 5 times, and followed by a centrifugation at 14,000 g for 5 min at 4° C. The resulting supernatant was immunoprecipitated with rabbit anti-CypD IgG (1:500) at 4° C. overnight followed by a second incubation with protein A/G (Pierce) for 2 hr at 20° C. The resultant immnoprecipitant was subjected to 10-20% Tis/Tricine SDS-PAGE. Western blotting was done by anti-Aβ IgG (6E10,1:3000, Signat). The same methodology was employed for studies on mitochondria derived from human brain tissues of AD patients and age-matched non-demented controls (N=9 in each group).

### Example 9

### Surface Plasmon Resonance Study of CypD-Aβ Interaction.

Surface Plasmon Resonance (SPR) has been employed in studying Aβ aggregation and Aβ-apoE and Aβ-ABAD interaction[9, 26, 27] and was performed as previously described [27] for studying CypD-Aβ interaction . Aβ40 and Aβ42 were obtained from rPeptide (www.rpeptide.com, catalog no. A-1156-2) or synthesis from the protein core of Yale University. The monomeric and oligomeric Aβ were prepared as described and characterized by atomic force microscopy [27]. SPR studies were performed on a BIAcore 3000 system (BIAcore AB, Uppsala, Sweden). Aβ (500 μg/ml) was immobilized using the standard amino coupling on a research-grade CM5 sensor chip. Various concentrations of CypD in the running buffer containing 50 mM Tris-HCL (pH 7.5) and 150 mM sodium chloride were injected at flow rate (40 μl/min)during the 90 s association phase, and chip surface was exposed to the running buffer for 120 s to monitor the dissociation phase. Data from a control well without Aβ immobilization or without the injection of CypD to the chip were subtracted from raw data. The binding curves were analyzed with the global fitting model using BIA evaluation version 4.0.1 (BIAcore AB).

### Example 10

### Immunostaining for Confocal and Electron Microscopy Study.

Brain sections from Tg mice and patients were doubly stained with goat anti-CypD (1:25, Sanda Cruz, Calif.) and rabbit anti-Aβ42 IgG (1:100, Biosource) followed by donkey anti-goat or donkey anti-rabbit antibody conjugated with FITC or rhodamine (1:100). Nuclei were visualized using fluorescent Nissl reagent (NeuroTrace 640/660 deep-red fluorescent Nissl stain, 1:150) (Molecular Probes). Images were examined under the confocal microscopy. Immunoelectron microscopy study was performed as described previously [8], ultrathin sections were incubated with rabbit anti-Aβ42 IgG antibody (0.5 μg/ml, Biosource) and goat-anti-CypD IgG overnight at 4° C., followed by donkey anti-rabbit and donkey anti-goat antibodies conjugated to colloidal gold (18 nm particle for Aβ42, 12 nm particle for CypD, 1:25; Jackson Laboratories, West Grove, Pa., and 12) for 1.5 h at room temperature. Sections were counterstained with uranyl acetate and examined by electron microscopy (JEOL 100S).

### Example 11

### Mitochondrial Function Assay

Oxygen consumption and activity of cytochrome c oxidase (COX IV) were measured in 12-month-old Tg mice as described previously [8]. Mitochondrial swelling assay was performed according to the method [28] with the modification. Mitochondria were isolated from the cortex of Tg mice, suspended in 1 ml swelling assay buffer (500 μg protein, 225 mM mannitol, 125 mM KCl, 1 mM succinate, 5 mM glutamate, 10 mM malate, pH 7.2) in the presence or absence of 1 mM CsA for 5 min on ice before experiment started. The mitochondrial swelling was triggered by 1 mM Pi and immediately recorded on an Amersham Biosciences Ultrospect 3100 pro spectrophotometer for 12 min.

To determine the effect of CypD deficiency on mitochondria swelling in response to exogenous Aβ42, mitochondria isolated from nonTg and Tg CypD−/− mice of 6-month-old were incubate with or without Aβ42 on ice for 10 min. CsA (1 mM) or vehicle was added to the mitochondria with Aβ42 for additional 5 min.

Mitochondrial membrane potential (mPT) was determined in primary cultured neuron derived from nonTg and Tg CypD−/− mice. The changes in response to the 12 hr treatment of 2 μM oligomeric Aβ or to the 1 hr treatment of 2 mM H2O2 were observed by TMRM staining using flow cytometry analysis.

Aβ- or H2O2-induced cytochrome c release was measured in the mitochondria isolated from nonTg and CypD-deficient mice. Briefly, mitochondria were incubated in the isolated buffer at 20° C. in the presence or absence of 500 μM H2O2 or 2 μM oligomeric Aβ for various time points. Then mitochondria were recovered by centrifuging at 14,000 g for 15 min. The resulting supernatant were collected and concentrated with Microcon centrifugal filter devices (Millipore). The corresponding volume of concentrated supernatant and mitochondria pellets were subject to 12% Bis-Tris SDS-PAGE and immunoblotting with mouse anti-cytochrome c IgG (1:2000).

ATP levels in the brain of Tg mice were determined using an ATP Bioluminesence Assay Kit (Roche) following the manufacture's instruction. Brain tissues were homogenized in the lysis buffer provided in the kit, incubated on ice for 15 min, and centrifuged at 14,000 g for 15 min. Subsequent supernatants were measured for the ATP levels using Luminescence plate reader (Molecular Devices) with an integration time of 10 sec.

### Example 12

### In Situ Detection of Mitochondrial ROS and Membrane Potential in Brain Slices

In situ measurements of ROS and mitochondrial membrane potential in transgenics brain slices were based on Murakami's report [29] with modifications. Experimental animals were anesthetized with ketamine (200 mg/kg) and xylazine (10 mg/kg) and killed by transcardial perfusion with cold PBS for 3 min. The brain was quickly removed and frozen in 2-methyl butane with dry ice. Coronal frozen brain sections of 10 μm thickness at the same level (bregma −2 mm, interaural 1.6 mm) were cut immediately and used to evaluate the in situ ROS and mitochondrial membrane potential. Frozen sections were treated with either 50 nM TMRM or 1 μM MitoSox in PBS for 15 min. After fixed in 4% formaldehyde in PBS for 15 min, the sections were washed in PBS and mounted. The images were taken under confocal microscope with a magnification of 600× immediately after the mounting. The intensity and area occupied of TMRM and MitoSox staining were analyzed by Universal image program.

### Example 13

### CypD Translocation

Experiment for CypD translocation to the inner membrane was performed as described by Friberg and Connern [17, 30]. Mitochondria were isolated from the cortex of mice, resuspended in the buffer (225 mM mannitol, 125 mM KCl, 1 mM succinate, 5 mM glutamate, 10 mM malate, 150 mM potassium thiocyanate, pH 7.2), and incubated on ice for 10 min in the presence of 1 mM Pi alone or with 0.8 μM Aβ. To determine the effect of CsA in CypD translocation, 1 mM CsA, or vehicle was added to the buffer during incubation. The inner membrane was obtained by a centrifugation at 150,000 g for 60 min and was subjected to the immunoblotting with anti-CypD IgG. Immunoblotting of mitochondrial fraction with anti-COX IV (1:4000) was employed as a control for the equal amount of mitochondrial protein used for the experiment.

### Example 14

### Neuronal Culture

Murine cortical neurons were cultured as described [8]. Briefly, murine cortex were dissected from Day 1 pups of nonTg and Tg CypD−/− mice, dissociated with 0.05% trypsin, and triturated in ice-cold Neurobasal A medium. Cells were then centrifuged at 200 g for 5 min to get rid of the debris. The resulting pellets were resuspended in culture medium (neurobasal A with 2% B27 supplement, 0.5 mM L-glutamine, 50 U/ml penicillin, and 50 μg/ml streptomycin) and plated onto poly-L-lysine-coated culture plates with an appropriate density. Aβ or H2O2 was added to the cultured medium at day 5 cultured neurons.

### Example 15

### Determination of Mitochondrial ROS, Cytochrome C Release, Membrane Potential, and Apoptosis in Cultured Neuron.

Primary cultured neurons derived from nonTg and CypD-deficient mice were incubated with 2 μM oligomeric Aβ, or H2O2 for various time points. The mitochondrial ROS was detected with 1 μM MitoSox staining The cytochrome c release was assessed by ELISA (Active Motif). Mitochondrial membrane potential was determined by TMRM labeling and analyzed by flow cytometry as described above. Apoptosis was assessed by TUNEL assay using in situ cell death detection kit (Roche). TUNEL-positive cells were detected under invert fluorescence microscope at the magnification of 400×.

### Example 16

### Behavioral and Neuropathological Analysis.

Behavioral studies were performed to assess spatial learning and memory in the radial arm water maze as previously described [9]. The four groups of animals under behavioral study were littermates and matched with gender to enhance the reproducibility and reliability of our results in the radial arm water maze. Investigators were unaware to mouse genotypes until behavioral test was done.

AChE activities in the hippocampus (subiculum) homogenates of Tg mice after behavioral test were measured by using Amplex Red Acetylcholinesterase assay kit (Molecular Probes, CA). Briefly, tissues were homogenized in RIPA (20 mM Tris, pH 7.5, 150 mM NaCl, 1% Nonidet P-40, 0.5% Sodium Deoxycholate, 1 mM EDTA 0.1% SDS) buffer with protease inhibitors (Calbiochem). Total 40 μg homogenate was diluted in reaction buffer to the final volume of 100 μl. Reaction was started by the addition of 100 μl working solution containing 400 μM Amplex Red, 2 U/ml HRP, 0.2 U/ml choline oxidase and 100 μM acetylcholine. The fluorescence was read at 560/590 nm in a Molecular Devices Gemini XPS fluorescence microplate reader.

### Example 17

### Generation of Human Mitochondrial CypD Protein.

A CypD-GST fusion protein construct was prepared by subcloning CypD cDNA (gene bank ACCESSION# BC005020) into the unique EcoRI and XhoI sites of the pGXE-4T vector to express a GST-CypD fusion protein. Following transformation of E. coli with this construct, GST-CypD protein was purified as described by the manufacturer (GE Healthcare Life Science). GST-CypD fusion protein was cleaved with thrombin and purified it as a human CypD protein.

### Example 18

### Generation and Characterization of TgmAPP/CypD−/− Mice

To determine the effect of CypD on Aβ-induced mitochondrial dysfunction, CypD homozygous null mice (CypD−/−) (Baines, C. P., et al., Loss of cyclophilin D reveals a critical role for mitochondrial permeability transition in cell death. Nature, 2005. 434(7033): p. 658-62.) and Tg mAPP expressing a mutant form of human APP under the control of PDGF-B chain promoter (Arancio, O., et al., RAGE potentiates Abeta-induced perturbation of neuronal function in transgenic mice. Embo J, 2004. 23(20): p. 4096-105, Lustbader, J. W., et al., ABAD directly links Abeta to mitochondrial toxicity in Alzheimer's disease. Science, 2004. 304(5669): p. 448-52), as previously reported, were employed to our studies. The Tg mAPP mouse model was well-suited to our strategy of determining whether absence of CypD protects from Aβ-mediated mitochondrial and neuronal dysfunction, and deficits in learning/memory, since these animals have been previously characterized with respect to changes in mitochondrial, neuropathologic, and behavioral endpoints (Caspersen, C., et al., Mitochondrial Abeta: a potential focal point for neuronal metabolic dysfunction in Alzheimer's disease. Faseb J, 2005. 19(14): p. 2040-1, Takuma, K., et al., ABAD enhances Abeta-induced cell stress via mitochondrial dysfunction. Faseb J, 2005. 19(6): p. 597-8). Further, increased expression of CypD was observed in Tg mAPP mice as animal age and accumulation of Aβ occurred in mitochondria (Lustbader and Caspersen supra). CypD−/− mice were crossed with Tg mAPP mice to produce four genotypes in the expected Mendelian ratio: single transgenics (Tg mAPP and Tg CypD−/−), double transgenics (Tg mAPP/CypD−/−), and non-transgenic littermate controls (nonTg). The genotypes of mice were identified by PCR. The absence of CypD protein in Tg CypD−/− mice and Tg mAPP/CypD−/− mice was verified by Western blotting with specific anti-CypD IgG. NonTg and Tg mAPP mice expressed CypD.

The invention is illustrated herein by the experiments described above and by the following examples, which should not be construed as limiting. The contents of all references, pending patent applications and published patents, cited throughout this application are hereby expressly incorporated by reference. Those skilled in the art will understand that this invention may be embodied in many different forms and should not be construed as limited to the embodiments set forth herein. Rather, these embodiments are provided so that this disclosure will fully convey the invention to those skilled in the art. Many modifications and other embodiments of the invention will come to mind in one skilled in the art to which this invention pertains having the benefit of the teachings presented in the foregoing description. Although specific terms are employed, they are used as in the art unless otherwise indicated. The diagnostic methods and therapies that require determining the level of CypD in a patient sample can be accomplished using the methods described here.

### Sequence Listing

| SEQ ID NO. 1, cDNA sequence for the human Cyclophylin D | |
|---|---|
| agacccgcgcgcgctgcagtcaaactcaagacacaagaggggcgggcacagggcgggctgggcgcgggcgctacgaccgc | |
| gacgcgacgccgagggcgaccgagccggacgagaggcagggcgcgaggcacggcgacgcggaggggcgccgggcgcggac | |
| gtcgttcccgaggccgctgggcaggagaaggaggaggaggcccttgggcgagcacatggacctgcacctgcggttgccct | |
| tcggcgagccggcgcaccacgacctcgacttccgtctacagcagggtttctgtcgactcttgaagtctcgggacacgtga | |
| ccactcttcccgaagccgatgtttccgaggtggaaggtgtcccactagggaaggaagtacacggtccgcccgctgaagtg | |
| gttggtgttaccgtgtccgcccttcaggtagatgccttcggcgaaaggactgctcttgaaatgtgacttcgtgcaccccg | |
| gtccacaggacaggtaccgattacgaccaggattgtggttgccgagggtcaagaagtagacgtggtatttctgtctgacc | |
| aacctaccgttcgtacaacacaagccagtgcagtttctcccgtacctgcagcacttcttttatcttagaaagccgagatt | |
| ctcaccctcctgtaggttcttctaacagtagtgtctgacaccggtcaactcgattagacaccggtcccacgaccgtacca | |
| ccgtcgacgtttacaggtacgtgggtccaccggcgcaacccgacagtcggttccacggactttgctatgcacacgggtga | |
| ggtgacagtgtcacacggactccttccgacgatccctacaatctggagccggtcctgggtggtgtaacgaaggattatgg | |
| gtgggaaggagtgctggagtaaagacccgtagaaacacctgtactacagtgggtggggaacagttcgtaacggacactaa | |
| cgggtcgggtctaagtagacacggaacctgtaccactaccactacccaacggtaggttcactttcagaaaaggaactggt | |
| tccccctgtcagtcaaaacgttttcctgagattatggacaaattataacagaaggattaaccctattaaattaattgttc | |
| taactgatcttcactttgacgttgtgattgaaggggcacgacaccacactggactcaaccactgtgtccggtgtctgggg | |
| tctcgaaccgaaaactttgtgttgagtcccgaaaacacttccaagggggcgactctagaaaggaggaccaatgacacttc | |
| ggacaaccaaacgacgacagcaaaaactcctcccgggtacccccatcctcgtcaacttggacccttgtttggagtgaact | |
| cgacacggatctgttacacttaaggacacaacgattgtcttcaccggacattcgaggacacgaggcctcccttcgtaaag | |
| gaccatccgaaactaaaaagacacacaatttctttaagttagatgagtactacacaatacgtattttgtaaagaccttgt | |
| acctaaacacaagtggaatttacacttttatttaggataaaagataccttttttttttttttttttttttt | |
| SEQ ID NO. 2 amino acid sequence for human Cyclophilin D | |
| MLALRCGSRW LGLLSVPRSV PLRLPAARAC SKGSGDPSSS SSSGNPLVYL DVDANGKPLG | 60 |
| RVVLELKADV VPKTAENFRA LCTGEKGFGY KGSTFHRVIP SFMCQAGDFT NHNGTGGKSI | 120 |
| YGSRFPDENF TLKHVGPGVL SMANAGPNTN GSQFFICTIK TDWLDGKHVV FGHVKEGMDV | 180 |
| VKKIESFGSK SGRTSKKIVI TDCGQLS | 207 |

SEQ ID NO. 3 amyloid beta binding region of Cyclophilin D, amino acids 97-119.
RVIPSFMCQAGDFTNHNGTGGKS

### REFERENCES

1. LaRue, B., E. Hogg, A. Sagare, S. Jovanovic, L. Maness, C. Maurer, R. Deane, and B. V. Zlokovic, Method for measurement of the blood-brain barrier permeability in the perfused mouse brain: application to amyloid-beta peptide in wild type and Alzheimer's Tg2576 mice. J Neurosci Methods, 2004. 138(1-2): p. 233-42.
2. Blass, J. P., Cerebrometabolic abnormalities in Alzheimer's disease. Neurol Res, 2003. 25(6): p. 556-66.
3. Blass, J. P., The mitochondrial spiral. An adequate cause of dementia in the Alzheimer's syndrome. Ann N Y Acad Sci, 2000. 924: p. 170-83.
4. Sheehan, J. P., R. H. Swerdlow, S. W. Miller, R. E. Davis, J. K. Parks, W. D. Parker, and J. B. Tuttle, Calcium homeostasis and reactive oxygen species production in cells transformed by mitochondria from individuals with sporadic Alzheimer's disease. J Neurosci, 1997. 17(12): p. 4612-22.
5. Swerdlow, R. H. and S. M. Khan, A “mitochondrial cascade hypothesis” for sporadic Alzheimer's disease. Med Hypotheses, 2004. 63(1): p. 8-20.
6. Shukkur, E. A., A. Shimohata, T. Akagi, W. Yu, M. Yamaguchi, M. Murayama, D. Chui, T. Takeuchi, K. Amano, K. H. Subramhanya, T. Hashikawa, H. Sago, C. J. Epstein, A. Takashima, and K. Yamakawa, Mitochondrial dysfunction and tau hyperphosphorylation in Ts1Cje, a mouse model for Down syndrome. Hum Mol Genet, 2006. 15(18): p. 2752-62.
7. Takuma, K., J. Yao, J. Huang, H. Xu, X. Chen, J. Luddy, A. C. Trillat, D. M. Stern, O. Arancio, and S. S. Yan, ABAD enhances Abeta-induced cell stress via mitochondrial dysfunction. Faseb J, 2005. 19(6): p. 597-8.
8. Caspersen, C., N. Wang, J. Yao, A. Sosunov, X. Chen, J. W. Lustbader, H. W. Xu, D. Stern, G. McKhann, and S. D. Yan, Mitochondrial Abeta: a potential focal point for neuronal metabolic dysfunction in Alzheimer's disease. Faseb J, 2005. 19(14): p. 2040-1.
9. Lustbader, J. W., M. Cirilli, C. Lin, H. W. Xu, K. Takuma, N. Wang, C. Caspersen, X. Chen, S. Pollak, M. Chaney, F. Trinchese, S. Liu, F. Gunn-Moore, L. F. Lue, D. G. Walker, P. Kuppusamy, Z. L. Zewier, O. Arancio, D. Stern, S. S. Yan, and H. Wu, ABAD directly links Abeta to mitochondrial toxicity in Alzheimer's disease. Science, 2004. 304(5669): p. 448-52.
10. Manczak, M., T. S. Anekonda, E. Henson, B. S. Park, J. Quinn, and P. H. Reddy, Mitochondria are a direct site of A beta accumulation in Alzheimer's disease neurons: implications for free radical generation and oxidative damage in disease progression. Hum Mol Genet, 2006. 15(9): p. 1437-49.
11. Wang, J., W. Tong, X. Zhang, L. Chen, Z. Yi, T. Pan, Y. Hu, L. Xiang, and Z. Yuan, Hepatitis C virus non-structural protein NS5A interacts with FKBP38 and inhibits apoptosis in Huh7 hepatoma cells. FEBS Lett, 2006. 580(18): p. 4392-400.
12. Crompton, M., Mitochondria and aging: a role for the permeability transition? Aging Cell, 2004. 3(1): p. 3-6.
13. Halestrap, A. P., G. P. McStay, and S. J. Clarke, The permeability transition pore complex: another view. Biochimie, 2002. 84(2-3): p. 153-66.
14. Halestrap, A., Biochemistry: a pore way to die. Nature, 2005. 434(7033): p. 578-9.
15. Crompton, M., S. Virji, and J. M. Ward, Cyclophilin-D binds strongly to complexes of the voltage-dependent anion channel and the adenine nucleotide translocase to form the permeability transition pore. Eur J Biochem, 1998. 258(2): p. 729-35.
16. Halestrap, A. P., K. Y. Woodfield, and C. P. Connern, Oxidative stress, thiol reagents, and membrane potential modulate the mitochondrial permeability transition by affecting nucleotide binding to the adenine nucleotide translocase. J Biol Chem, 1997. 272(6): p. 3346-54.
17. Connern, C. P. and A. P. Halestrap, Recruitment of mitochondrial cyclophilin to the mitochondrial inner membrane under conditions of oxidative stress that enhance the opening of a calcium-sensitive non-specific channel. Biochem J, 1994. 302 (Pt 2): p. 321-4.
18. Andreeva, L., R. Heads, and C. J. Green, Cyclophilins and their possible role in the stress response. Int J Exp Pathol, 1999. 80(6): p. 305-15.
19. Baines, C. P., R. A. Kaiser, N. H. Purcell, N. S. Blair, H. Osinska, M. A. Hambleton, E. W. Brunskill, M. R. Sayen, R. A. Gottlieb, G. W. Dorn, J. Robbins, and J. D. Molkentin, Loss of cyclophilin D reveals a critical role for mitochondrial permeability transition in cell death. Nature, 2005. 434(7033): p. 658-62.
20. Pastorino, J. G., S. T. Chen, M. Tafani, J. W. Snyder, and J. L. Farber, The overexpression of Bax produces cell death upon induction of the mitochondrial permeability transition. J Biol Chem, 1998. 273(13): p. 7770-5.
21. Nakagawa, T., S. Shimizu, T. Watanabe, O. Yamaguchi, K. Otsu, H. Yamagata, H. Inohara, T. Kubo, and Y. Tsujimoto, Cyclophilin D-dependent mitochondrial permeability transition regulates some necrotic but not apoptotic cell death. Nature, 2005. 434(7033): p. 652-8.
22. Masliah, E., R. D. Terry, R. M. DeTeresa, and L. A. Hansen, Immunohistochemical quantification of the synapse-related protein synaptophysin in Alzheimer disease. Neurosci Lett, 1989. 103(2): p. 234-9.
23. Geula, C., Abnormalities of neural circuitry in Alzheimer's disease: hippocampus and cortical cholinergic innervation. Neurology, 1998. 51(1 Suppl 1): p. S18-29; discussion S65-7.
24. Yan, S. D. and D. M. Stern, Mitochondrial dysfunction and Alzheimer's disease: role of amyloid-beta peptide alcohol dehydrogenase (ABAD). Int J Exp Pathol, 2005. 86(3): p. 161-71.
25. Arancio, O., H. P. Zhang, X. Chen, C. Lin, F. Trinchese, D. Puzzo, S. Liu, A. Hegde, S. F. Yan, A. Stern, J. S. Luddy, L. F. Lue, D. G. Walker, A. Roher, M. Buttini, L. Mucke, W. Li, A. M. Schmidt, M. Kindy, P. A. Hyslop, D. M. Stern, and S. S. Du Yan, RAGE potentiates Abeta-induced perturbation of neuronal function in transgenic mice. Embo J, 2004. 23(20): p. 4096-105.
26. Aguilar, M. I. and D. H. Small, Surface plasmon resonance for the analysis of beta-amyloid interactions and fibril formation in Alzheimer's disease research. Neurotox Res, 2005. 7(1-2): p. 17-27.
27. Yan, Y., Y. Liu, M. Sorci, G. Belfort, J. W. Lustbader, S. S. Yan, and C. Wang, Surface plasmon resonance and nuclear magnetic resonance studies of ABAD-Abeta interaction. Biochemistry, 2007. 46(7): p. 1724-31.
28. Schinzel, A. C., O. Takeuchi, Z. Huang, J. K. Fisher, Z. Zhou, J. Rubens, C. Hetz, N. N. Danial, M. A. Moskowitz, and S. J. Korsmeyer, Cyclophilin D is a component of mitochondrial permeability transition and mediates neuronal cell death after focal cerebral ischemia. Proc Natl Acad Sci USA, 2005. 102(34): p. 12005-10.
29. Murakami, K., T. Kondo, M. Kawase, Y. Li, S. Sato, S. F. Chen, and P. H. Chan, Mitochondrial susceptibility to oxidative stress exacerbates cerebral infarction that follows permanent focal cerebral ischemia in mutant mice with manganese superoxide dismutase deficiency. J Neurosci, 1998. 18(1): p. 205-13.
30. Friberg, H., C. Connern, A. P. Halestrap, and T. Wieloch, Differences in the activation of the mitochondrial permeability transition among brain regions in the rat correlate with selective vulnerability. J Neurochem, 1999. 72(6): p. 2488-97.
31. Halestrap, A. P., et al., Elucidating the molecular mechanism of the permeability transition pore and its role in reperfusion injury of the heart. Biochim Biophys Acta, 1998. 1366(1-2): p. 79-94.
32. Morais Cardoso, S., R. H. Swerdlow, and C. R. Oliveira, Induction of cytochrome c-mediated apoptosis by amyloid beta 25-35 requires functional mitochondria. Brain Res, 2002. 931(2): p. 117-25.
33. Gelain, D. P., et al., Retinol induces the ERK1/2-dependent phosphorylation of CREB through a pathway involving the generation of reactive oxygen species in cultured Sertoli cells. Cell Signal, 2006. 18(10): p. 1685-94.
34. Maragos, W. F., et al., Methamphetamine toxicity is attenuated in mice that overexpress human manganese superoxide dismutase. Brain Res, 2000. 878(1-2): p. 218-22.
35. Hensley, K., et al., A model for beta-amyloid aggregation and neurotoxicity based on free radical generation by the peptide: relevance to Alzheimer disease. Proc Natl Acad Sci USA, 1994. 91(8): p. 3270-4.
36. Serrano, F. and E. Klann, Reactive oxygen species and synaptic plasticity in the aging hippocampus. Ageing Res Rev, 2004. 3(4): p. 431-43.
37. van Loo, G., et al., Inhibition of transcription factor NF-kappaB in the central nervous system ameliorates autoimmune encephalomyelitis in mice. Nat Immunol, 2006. 7(9): p. 954-61.
38. Liu, R., et al., Reversal of age-related learning deficits and brain oxidative stress in mice with superoxide dismutase/catalase mimetics. Proc Natl Acad Sci USA, 2003. 100(14): p. 8526-31.
39. Esposito, L., et al., Reduction in mitochondrial superoxide dismutase modulates Alzheimer's disease-like pathology and accelerates the onset of behavioral changes in human amyloid precursor protein transgenic mice. J Neurosci, 2006. 26(19): p. 5167-79.
40. Xie, Y., et al., Identification of small-molecule inhibitors of the Abeta-ABAD interaction. Bioorg Med Chem Lett, 2006. 16(17): p. 4657-60.
41. Origlia, N., et al., Receptor for advanced glycation end product-dependent activation of p38 mitogen-activated protein kinase contributes to amyloid-beta-mediated cortical synaptic dysfunction. J Neurosci, 2008. 28(13): p. 3521-30.

1. -48. (canceled)

49. A method for treating or preventing Alzheimer's disease or Parkinson disease in a subject by administering a therapeutically effective amount of cyclosporine, or a biologically active analog, derivative, variant or fragment thereof; or an antibody or a biologically active fragment thereof that specifically binds to cyclophilin D thereby reducing its biological activity; or an agent that reduces expression of cyclophilin D, or combinations thereof.

50. The method of claim 49, wherein the cyclosporine is a member selected from the group comprising cyclosporine A, cyclosporine D, and NIM811.

51. The method of claim 49, wherein disease is Alzheimer's disease and the antibody specifically binds to all or part of the amyloid beta-binding region of cyclophilin D identified herein as amino acid SEQ ID NO. 3.

52. The method of claim 49, wherein the antibody is a selected from the group comprising a polyclonal antibody, a monoclonal antibody, and the SDY-1 antibody, preferably in humanized form.

53. The method of claim 49, wherein the agent that reduces the expression of cyclophilin D is an antisense nucleotide or small interfering RNA, that is sufficiently complementary to the gene or mRNA encoding cyclophilin D to permit specific hybridization to the gene or mRNA, respectively.

54. The method as in claim 49, wherein the subject is a human.

55. A method of diagnosing and treating or preventing Alzheimer's disease or Parkinson disease in a subject, comprising:
a. determining a patient level of cyclophilin D in a biological sample taken from the patient and a control level of cyclophilin D in a biological sample taken from a control subject that is not afflicted with the disease,
b. comparing the patient and control levels, and
c. if the patient level is significantly higher than the control level, then administering a therapeutically effective amount of cyclosporine, or a biologically active analog, derivative, variant or fragment thereof; an antibody or biologically active fragment thereof that specifically binds to cyclophilin D thereby reducing its biological activity; or an agent that reduces the expression of cyclophilin D or combinations thereof.

56. The method of claim 55, wherein the cyclosporine is a member selected from the group comprising cyclosporine A, cyclosporine D, and NIM811.

57. The method of claim 55, wherein the antibody specifically binds to all or part of the amyloid beta-binding region of cyclophilin D identified herein as amino acid SEQ ID NO. 3.

58. The method of claim 55, wherein the antibody is a selected from the group comprising a polyclonal antibody, a monoclonal antibody, the SDY-1 antibody, preferably in humanized form.

59. The method of claim 55, wherein the agent that reduces the expression of cyclophilin D is an antisense nucleotide or small interfering RNA that is sufficiently complementary to the gene or mRNA encoding cyclophilin D to permit specific hybridization to the gene or mRNA, respectively.

60. The method as in claim 55, wherein the subject is a human.

61. A cyclophilin polypeptide fragment comprising the amino acid sequence identified by SEQ ID NO. 3.

62. A method of treating or preventing Alzheimer's disease in a patient, comprising administering an antibody or biologically active fragment or variant thereof that specifically binds to amyloid beta, which antibody prevents formation of a complex of amyloid beta and cyclophilin D.

63. A method for reducing memory loss associated with aging, Alzheimer's disease or Parkinson disease, by administering a therapeutically effective amount of cyclosporine, or a biologically active analog, derivative, variant or fragment thereof; an antibody or a biologically active variant or fragment thereof that specifically binds to cyclophilin D thereby reducing its biological activity; or an agent that reduces the expression of cyclophilin D, or a combination thereof.

64. The method of claim 63, wherein the cyclosporine is a member selected from the group comprising cyclosporine A, cyclosporine D, and NIM811.

65. A diagnostic kit for diagnosing a subject at risk of developing or having Alzheimer's Disease, Parkinson Disease or other disease associated with abnormally elevated cyclophilin D expression, comprising an antibody that specifically binds to cyclophilin D, and reagents for detection of the antibody.

66. The diagnostic kit of claim 65, wherein the kit contains reagents for detection of the antibody by an enzyme-linked immunosorbent assay.

67. A pharmaceutical composition for treating or preventing Alzheimer's disease or Parkinson disease in a subject, comprising a therapeutically effective amount of cyclosporine, or a biologically active analog, derivative, variant or fragment thereof and an antibody or a biologically active fragment thereof that specifically binds to cyclophilin D thereby reducing its biological activity.

68. A pharmaceutical composition for treating or preventing Alzheimer's disease or Parkinson disease in a subject, comprising a therapeutically effective amount of cyclosporine, or a biologically active analog, derivative, variant or fragment thereof and an agent that reduces expression of cyclophilin D.

69. The pharmaceutical composition of claim 67, further comprising an agent that reduces expression of cyclophilin D.

70. The pharmaceutical formulation of claim 69, further comprising an antibody or a biologically active fragment thereof that specifically binds to cyclophilin D thereby reducing its biological activity.

71. An antibody that specifically binds to cyclophilin D at a site that prevents formation of the cyclophilin D amyloid beta complex.

72. The antibody of claim 71, wherein the antibody specifically binds to all or part of the amyloid beta-binding region of cyclophilin D identified herein as amino acid SEQ ID NO. 3.

73. The antibody of claim 71, wherein the antibody is the polyclonal antibody described herein and identified as SDY-1.

74. The antibody of claim 71, wherein the antibody is humanized.


SEQUENCE LISTING (7 sequences)

SEQ ID NO: 1 - DNA, 1591 bp, Homo sapiens
agacccgcgcgcgctgcagtcaaactcaagacacaagaggggcgggcacagggcgggctgggcgcgggcgctacgaccgcgacgcgacgccgagggcgaccgagccggacgagaggcagggcgcgaggcacggcgacgcggaggggcgccgggcgcggacgtcgttcccgaggccgctgggcaggagaaggaggaggaggcccttgggcgagcacatggacctgcacctgcggttgcccttcggcgagccggcgcaccacgacctcgacttccgtctacagcagggtttctgtcgactcttgaagtctcgggacacgtgaccactcttcccgaagccgatgtttccgaggtggaaggtgtcccactagggaaggaagtacacggtccgcccgctgaagtggttggtgttaccgtgtccgcccttcaggtagatgccttcggcgaaaggactgctcttgaaatgtgacttcgtgcaccccggtccacaggacaggtaccgattacgaccaggattgtggttgccgagggtcaagaagtagacgtggtatttctgtctgaccaacctaccgttcgtacaacacaagccagtgcagtttctcccgtacctgcagcacttcttttatcttagaaagccgagattctcaccctcctgtaggttcttctaacagtagtgtctgacaccggtcaactcgattagacaccggtcccacgaccgtaccaccgtcgacgtttacaggtacgtgggtccaccggcgcaacccgacagtcggttccacggactttgctatgcacacgggtgaggtgacagtgtcacacggactccttccgacgatccctacaatctggagccggtcctgggtggtgtaacgaaggattatgggtgggaaggagtgctggagtaaagacccgtagaaacacctgtactacagtgggtggggaacagttcgtaacggacactaacgggtcgggtctaagtagacacggaacctgtaccactaccactacccaacggtaggttcactttcagaaaaggaactggttccccctgtcagtcaaaacgttttcctgagattatggacaaattataacagaaggattaaccctattaaattaattgttctaactgatcttcactttgacgttgtgattgaaggggcacgacaccacactggactcaaccactgtgtccggtgtctggggtctcgaaccgaaaactttgtgttgagtcccgaaaacacttccaagggggcgactctagaaaggaggaccaatgacacttcggacaaccaaacgacgacagcaaaaactcctcccgggtacccccatcctcgtcaacttggacccttgtttggagtgaactcgacacggatctgttacacttaaggacacaacgattgtcttcaccggacattcgaggacacgaggcctcccttcgtaaaggaccatccgaaactaaaaagacacacaatttctttaagttagatgagtactacacaatacgtattttgtaaagaccttgtacctaaacacaagtggaatttacacttttatttaggataaaagataccttttttttttttttttttttttt

SEQ ID NO: 2 - protein, 207 aa, Homo sapiens
MLALRCGSRWLGLLSVPRSVPLRLPAARACSKGSGDPSSSSSSGNPLVYLDVDANGKPLGRVVLELKADVVPKTAENFRALCTGEKGFGYKGSTFHRVIPSFMCQAGDFTNHNGTGGKSIYGSRFPDENFTLKHVGPGVLSMANAGPNTNGSQFFICTIKTDWLDGKHVVFGHVKEGMDVVKKIESFGSKSGRTSKKIVITDCGQLS

SEQ ID NO: 3 - protein, 23 aa, Homo sapiens
RVIPSFMCQAGDFTNHNGTGGKS

SEQ ID NO: 4 - protein, 11 aa, Human immunodeficiency virus type 1
YGRKKRRQRRR

SEQ ID NO: 5 - DNA, 19 bp, Artificial Sequence
  Synthetic sequence
gcacaggagggaggtccat

SEQ ID NO: 6 - DNA, 19 bp, Artificial Sequence
  Synthetic sequence
gccccacatgcttcagtgt

SEQ ID NO: 7 - DNA, 16 bp, Artificial Sequence
  Synthetic sequence
aagccgctttcccgac